(19) United States
(12) Patent Application Publication
(10) Pub. No.: US 2011/0009584 A1
(43) Pub. Date: Jan. 13, 2011

(54) Romp-Polymerizable Electron Transport Materials Based On A Bis-Oxadiazole Moiety

(75) Inventors: Seth Marder, Atlanta, GA (US); Stephen Barlow, Atlanta, GA (US); Yadong Zhang, Alpharetta, GA (US); Sushanta Pal, Riverside, CA (US); Bernard Kippelen, Decatur, GA (US); Benoit Domerq, Waterloo (BE); Andreas Haldi, Dresden (DE); Marcus Weck, New York, NY (US); Alpay Kimyonok, Besiktas Istanbul (TR)

Correspondence Address: Solvay;c/o B. Ortego - IAM-NAFTA, 3333 Richmond Avenue, Houston, TX, 77098-3099, US

(73) Assignee: GEORGIA TECH RESEARCH CORPORATION, Atlanta, GA (US)

(21) Appl. No.: 12/808,743

(22) PCT Filed: Dec. 19, 2008
(86) PCT No.: PCT/EP2008/068119

§ 371 Date: Aug. 16, 2010

Related U.S. Application Data
(60) Provisional application No. 61/015,777, filed on Dec. 21, 2007.

(51) Int. Cl. C08F 26/06; C07D 413/10

(52) U.S. Cl. 526/260; 548/131

(57) ABSTRACT

This invention relates generally to a solution processable norbornene monomer, poly(norbornene) homopolymer, and poly(norbornene) copolymer compounds containing a functionalized bis-oxadiazole side chain, and to an electron injecting/transporting layer, a hole-blocking layer, or an emissive material, organic electronic devices and compositions which include these compounds.

### RELATED APPLICATIONS

This application claims the priority of U.S. Provisional Application No. 61,015, 777 filed Dec. 21, 2007. The entire disclosure of the predecessor application is hereby incorporated herein by reference in its entirety.

### STATEMENT REGARDING FEDERALLY SPONSORED RESEARCH OR DEVELOPMENT

This invention was made with government support under a grant from the Office of Naval Research, Grant Nos. 68A-1060806. The U.S. Government has certain rights in this invention.

### FIELD OF THE INVENTION

This invention relates generally to norbornene monomer, poly(norbornene) homopolymer, and poly(norbornene) copolymer compounds containing a functionalized bis-oxadiazole side chain, and to electron injecting/transporting and/or hole-blocking layers, electron transport emissive materials, and host materials for an organic luminescence layer, organic electronic devices, and compositions which include these compounds.

### BACKGROUND OF THE INVENTION

In recent years, much research has focused on the development of polymeric materials for application in electro-optic devices and organic light emitting diodes. Monomeric oxadiazoles can have effective electron-injecting and transporting functions, exhibit hole-blocking properties, and can also serve as hosts for phosphorescent organic light emitting diodes. C. Adachi, et al., Appl. Phys. Lett., 1990, 56, 799; G. Hughes, et al., Mater. Chem., 2005, 15, 94; Michikawa, et al., J. Mater. Chem., 2006, 16, 221; and M. K. Leung, et al., Org. Lett. 2007, 9, 235.

Since the initial studies using the oxadiazole small molecule 2-(4-biphenyl)-5-(4-tert-butylphenyl)-1,3,4-oxadiazole (PBD), shown below, monomeric 2,5-diaryl-1,3,4-oxadiazoles have been used as electron-transporting and hole-blocking materials in OLED devices due to their electron-accepting nature, and their high thermal stability. Their high photoluminescence quantum yield has also led to their use as the emissive component of OLED s. However, vacuum-evaporated amorphous thin films of PBD have been found to crystallize over time, due to joule heating during device operation. This crystallization results in reduced device lifetimes.

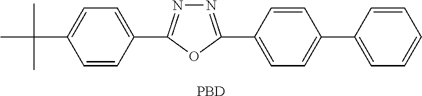

Small oxadiazole molecules have also been developed as an electron transporting host for phosphorescent organic light-emitting devices. Hole-blocking materials based on oxadiazoles have also developed. Nevertheless, there is a need for improved electron transporting and/or hole blocking materials with improved properties and improved processability.

### SUMMARY OF THE INVENTION

An object of the present invention is to provide a solution processable norbornene monomers, poly(norbornene) homopolymers, and poly(norbornene) copolymer compounds containing a functionalized bis-oxadiazole side chain, and to provide electron injecting/transporting and/or hole blocking layers, electron transport emissive materials, and host materials for an organic luminescence layer, organic electronic devices and compositions of matter which include these compounds.

In accordance with the purpose(s) of this invention, as embodied and broadly described herein, in some aspects these inventions relate to a compound within the scope of formula (I):

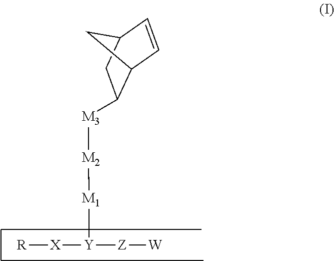

wherein:

R and W are aryl groups that will be further described below;

X and Z comprise oxadiazoles;

Y is absent or arene diyl;

the R—X—Y—Z—W unit taken together is linked to the norbornene monomer by a M₁-M₂-M₃ linker groups, wherein the identities of M₁, M₂, and M₃ groups will be further described below.

In other aspects, the inventions relate to polymers or copolymers comprising monomer units within the scope of formula II:

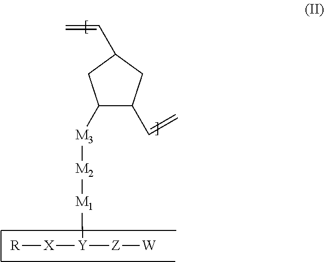

wherein R, X, Y, Z, W, M₁, M₂, and M₃ are described herein. In related aspects, the inventions relate to electron injecting/transporting and/or hole blocking layers, electron transport emissive materials, and host materials for comprising the monomers of formula I or the polymers and copolymers of formula II for use in organic electronic devices.

### DESCRIPTION OF THE FIGURES

FIG. 1—Diagram of device configuration of Example 17.

FIG. 2—Current density-Voltage (J-V) characteristics for OLED devices of Example 17.

FIG. 3—Maximum luminance and external quantum efficiency (EQE) as a function of voltage for the OLED devices of Example 17.

FIG. 4—Diagram of device configuration of Example 18.

FIG. 5—Maximum luminance and external quantum efficiency (EQE) as a function of voltage for the OLED devices of Example 18.

FIG. 6—Diagram of device configuration of Example 19.

FIG. 7—Maximum luminance and external quantum efficiency (EQE) as a function of voltage for the OLED devices of Example 19.

FIG. 8—Diagram of device configuration of Example 20.

FIG. 9—Current density-Voltage (J-V) characteristics for OLED devices of Example 20.

FIG. 10—Maximum luminance and external quantum efficiency (EQE) as a function of voltage for the OLED devices of Example 20.

FIG. 11—Diagram of device configuration of Example 21.

FIG. 12—Current density-Voltage (J-V) characteristics for OLED devices of Example 21.

FIG. 13—Maximum luminance and external quantum efficiency (EQE) as a function of voltage for the OLED devices of Example 21.

FIG. 14—Diagram of device configuration of Example 22.

FIG. 15—Current density-Voltage (J-V) characteristics for OLED devices of Example 22.

FIG. 16—Maximum luminance and external quantum efficiency (EQE) as a function of voltage for the OLED devices of Example 22.

### DETAILED DESCRIPTION OF THE INVENTION

The present invention may be understood more easily by reference to the following detailed description of preferred embodiments of the invention and the Examples included therein.

The invention concerns a novel type of oxadiazole monomer in which a bis-oxadiazole is covalently linked to a polymerizable norbornene group, along with homo and copolymers of these monomers. These materials may function as electron-transporting, hole-blocking, energy transfer host and/or luminescent functional moieties. Conjugated polymers containing the phenyl-oxadiazole unit are of great interest because they are thermally stable and possess extremely interesting electro-optical and electronic properties. When compared to small oxadiazole molecules, oxadiazole-containing polymers can be readily processed into amorphous thin films by wet processing methods such as spin-coating and printing, thus facilitating the low cost fabrication of OLEDs.

We have been successful in developing novel compounds: bis-oxadiazole monomers and related polymers where the M₁, R, X, Y, Z, and W groups are non-linearly disposed and/or optionally substituted to improve the solubility and processability of the monomeric and polymeric compounds.

### Definitions

Before the present compounds, compositions, articles, devices, and or methods are disclosed and described, it is to be understood that the terminology used herein is for the purpose of describing particular embodiments only and is not intended to be limiting.

It must be noted that as used in the specification and the appended claims, the singular forms “a” “an” and “the” include plural referents unless the context clearly dictates otherwise. Thus, for example, reference to “a cyclic compound” includes mixtures of aromatic compounds.

In the specification and claims which follow, reference will be made to a number of terms which shall be defined to have the following meanings:

Ranges are often expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment.

The term “halogen” and “halo” refer to bromine, chlorine, fluorine and iodine.

The term “alkoxy” refers to a straight, branched or cyclic C₁₋₂₀ alkyl-O, with the alkyl group optionally substituted as described herein.

The term “diyl” refers to a group of atoms attached to two other groups of atoms in two places.

The terms “alkanediyl” or “alkane diyl” refers to a straight chain, branched chain or cyclic alpha, omega-alkanediyl having a carbon chain length of from 1 to 20 carbon atoms, such as methane diyl, ethane diyl, propane diyl and the like.

The terms “alkenediyl” or “alkene diyl” refers to a straight chain, branched chain or cyclic alpha, omega-alkenediyl having a carbon chain length of from 1 to 20 carbon atoms, such as ethenediyl, propenediyl, butanediyl and the like.

The terms “alkynediyl” or “alkynediyl” refers to a straight chain, branched chain or cyclic alpha, omega-alkynediyl having a carbon chain length of from 1 to 20 carbon atoms, such as ethynediyl, propynediyl, butynediyl and the like.

The term “arenediyl” refers to an aromatic or heteroaromatic aryl group where two hydrogen atoms are removed allowing for a group to be substituted at the position where the two hydrogen atoms were removed, and having a chain length from 1 to 20 carbon atoms.

The term “alkyl” refers to a branched or straight chain saturated hydrocarbon group, having a carbon chain length of from 1 to 20 carbon atoms, such as methyl, ethyl, propyl, n-propyl, isopropyl, butyl, n-butyl, isobutyl, t-butyl, octyl, decyl, decyl, tetradecyl, hexadecyl, eicosyl, tetracosyl, cyclopentyl, cyclohexyl and the like. When substituted, alkyl groups may be substituted with at least one member selected from the group consisting of CN, NO₂, S, NH, OH, COO—, and halogen at any available point of attachment. When the alkyl group is said to be substituted with an alkyl, this is used interchangeably with “branched alkyl” group.

The term “alkenyl” refers to a hydrocarbon radical straight, branched or cyclic containing 2 to 10 carbon atoms and at least one carbon to carbon double bond. Suitable alkenyl groups include ethenyl, propenyl, butenyl and cyclohexenyl.

The term “alkynyl” refers to a hydrocarbon radical straight or branched, containing from 2 to 10 carbon atoms and at least one carbon to carbon triple bond. Preferred alkynl groups include ethynyl, propynyl and butynyl.

The terms “cyclic” and “aryl” refer to aromatic rings, e.g. phenyl, substituted phenyl, benzene and the like as well as rings which are fused, e.g. naphthyl, phenanthrenyl, and the like. A cyclic or aryl group thus contains at least one ring having at least 6 atoms. Substituents on the cyclic or aryl group may be present on any position, i.e., ortho, meta, or para positions or fused to the aromatic ring. Suitable cyclic or aryl groups are phenyl, naphthyl, and phenanthrenyl and the like. More particularly, cyclic or aryl groups may be unsubstituted or substituted with an aromatic or heteroaromatic group, and the aromatic or heteroaromatic group may be substituted with a substituent independently selected from the group consisting of a different aryl group, alkyl groups, halogens, fluoroalkyl groups; alkoxy groups, and amino groups. Preferred substituted aryl or cyclic groups include phenyl, naphthyl and the like.

The terms “heterocyclic” or “heteroaryl” refer to a conjugated monocyclic aromatic hydrocarbon group having 5 or 6 ring atoms, a conjugated bicyclic aromatic group having 8 to 10 atoms, or a conjugated polycyclic aromatic group having at least 12 atoms, containing at least one heteroatom, O, S, or N, in which a C or N atom is the point of attachment, and in which 1 or 2 additional carbon atoms is optionally replaced by a heteroatom selected from O, or S, and in which from 1 to 3 additional carbon atoms are optionally replaced by nitrogen heteroatoms, said heteroaryl group being optionally substituted as described herein. Examples of this type are pyrrole, oxazole, thiazole, pyridyl and oxazine. Additional nitrogen atoms may be present together with the first nitrogen and oxygen or sulfur, giving, e.g. thiadiazole. Suitable heterocyclic compounds are oxadiazole, purine, indole, purine, pyridyl, pyrimidine, pyrrole, imidazole, thiazole, oxazole, furan, thiophene, triazole, pyrazole, isoxazole, isothiazole, pyrazine, pyridazine, and triazine.

The term “oxadiazole” as used herein is meant to describe a 1-oxa-3,4-diazol-2,5-diyl group as shown below:

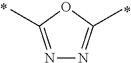

The asterisk (*) used herein is intended to denote the point of attachment on the chemical structure.

The subscript “n” refers to the number of repeat units in the polymer.

The Monomeric Oxadiazoles

Many embodiments of the present inventions relate to compounds represented by the formula (I):

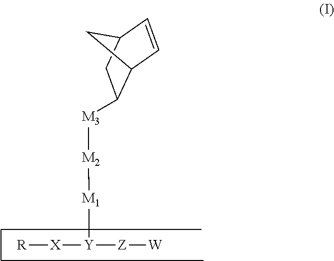

wherein:

R and W are each aryls and are optionally substituted;

X and Z are each oxadiazole;

Y is absent or arenediyl;

wherein R—X—Y—Z—W taken together is a unit that is linked to the norbornene monomer by a linkage M₁-M₂-M₃, and wherein the linkage is attached to one of Y or W;

M₁ and M₃ are independently absent or represent

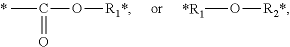

and is attached to the R—X—Y—Z—W unit through the carbon or oxygen atom on the ester, or through the ether oxygen atom, and M₂ is R₃;

R₁ and R₂ are independently absent or selected from the group consisting of alkane diyl, alkene diyl, alkynediyl, and arenediyl, each of which are straight chain, branched chain or cyclic, having a carbon chain length of from 1 to 20 carbon atoms; and

R₃ is absent or represents alkane diyl, alkene diyl, alkynediyl, or arenediyl, each of which are straight chain, branched chain or cyclic, having a carbon chain length of from 1 to 20 carbon atoms.

We have discovered that the solubilities and/or processability of the monomeric and polymeric compounds are significantly improved if the M₁, R, X, Y, Z, and W moieties are linked so as to form a non-linear geometry along the backbone of the M₁, R, X, Y, Z, and W moiety. More particularly, when the two oxadiazole X and Z groups are non-linearly positioned with respect to the Y group, soluble bis-oxadiazoles are usually obtained. If the two X and Z oxadiazole groups are linearly attached through the Y, group, the solubility can be improved by attaching the M₁ group in a position that induces a non-linear geometry in the molecules.

For example the carbazole monomers of the invention can be represented by the formula Ia:

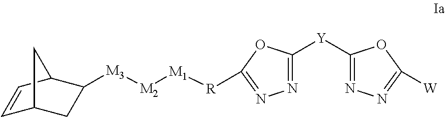

Preferably, the substitution geometries around the R and/or Y groups are not linear, which can substantially improve the solubility and/or processability of the resulting compounds Ia, at least as compared to compounds where the geometries around both R and Y are linear.

In formulas I and Ia, Y can be absent or is C₆-C₂₀ arene For example, Y can be any of the following substituted or unsubstituted rings: phenyl, naphthyl, anthracenyl, fluorenyl, phenanthrenyl, pyridyl or biphenyl.

Y can preferably be a phenyl group, especially the m-phenyl groups as shown below:

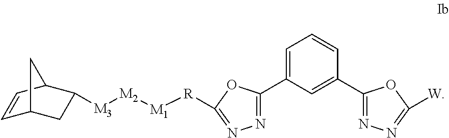

In formulas I and Ia, R can be an arene comprising six to twenty carbon atoms optionally substituted with 1, 2, or 3 independently select ed alkyl or alkoxy groups. For example, R can be any of the following substituted or unsubstituted rings: phenyl, naphthyl, anthracenyl, fluorenyl, phenanthrenyl, pyridyl or biphenyl.

R can preferably be a phenyl as shown below:

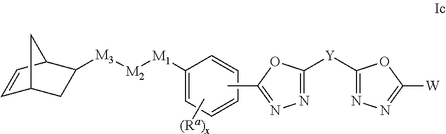

where each optional R^{a} group can be C₁₋₂₀ alkyl, or alkoxy groups, and x is an integer 1, 2, or 3. Preferably, the oxadiazole ring is not disposed on the phenyl ring at the para position of the optionally substituted benzene group.

In formulas I and Ia, W can be an arene comprising six to twenty carbon atoms optionally substituted with 1, 2, or 3 independently selected alkyl or alkoxy groups. For example, W can be any of the following substituted or unsubstituted rings: phenyl, naphthyl, anthracenyl, fluorenyl, phenanthrenyl, pyridyl or biphenyl.

W can preferably be a phenyl as shown below:

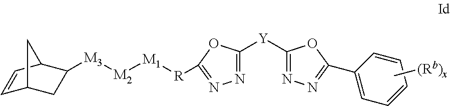

where each optional R^{b} group can be one or more C₁₋₂₀ alkyl or alkoxy groups, and x is an integer 1, 2, or 3. In a relate d embodiment, R^{b} can be a tert-butyl group. In another related embodiment, R^{b} can be *—O—(CH₂)₂CH₃, where z is an integer 0, 1, 2, 3, 4, 5, 6, 7, 8, 9, 10, 11, or 12, and R^{b} is bound to the phenyl at the position indicated by *.

In a related embodiment of the invention, both R and W can be phenyl as shown below:

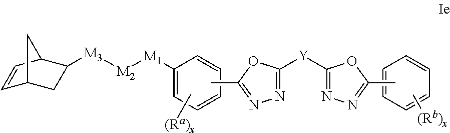

Where each optional R^{a} or R^{b} are as described above for formulas Ic and Id. In related embodiments of the invention, the M₃-M₂-M₁ is linker is connected through the Y group as shown below:

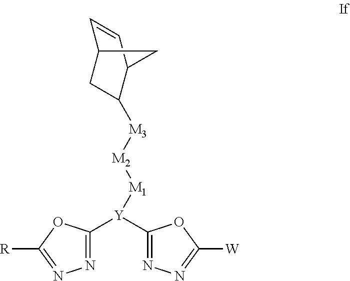

where R, W, Y, M₁ and M₃, are as described herein.

In formulas I, Ia, Ib, Ic, Id, Ie, and If, M₁ and M₃ an be optional or independently selected from

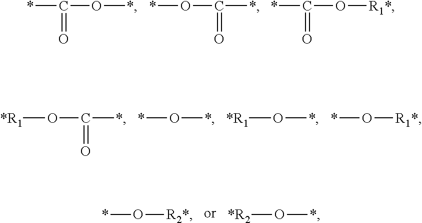

where M₁ and M₃ are bound to the norbornene or R at the positions indicated by *.

In some embodiments, M₂ can be absent. In other embodiments, M₂ can be *—(CH₂)_{z}—* where z is an integer 1, 2, 3, 4, 5, 6, 7, 8, 9, 10, or 11. In another related embodiment, M₃-M₂-M₁ taken together can be

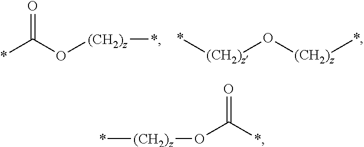

or *—(CH₂)_{z}—* where z can be an integer 0, 1, 2, 3, 4, 5, 6, 7, 8, 9 or 10.

In formula I, Ia, Ib, Ic, Id, Ie, and If, R₁ and R₂ are optional independently selected C₁₋₂₀ alkane diyl, alkene diyl, alkyne diyl, or arene diyl groups. In some related embodiments, R₁ and R₂ can be —(CH₂)_{z}— where z is an independently selected integer 0, 1, 2, 3, 4, 5, 6, 7, 8, 9, 10, 11, or 12. In another related embodiment, R₁ and R₂ are absent.

In related embodiments, the inventions relate to the following novel substituted norbornenyl monomeric compounds:

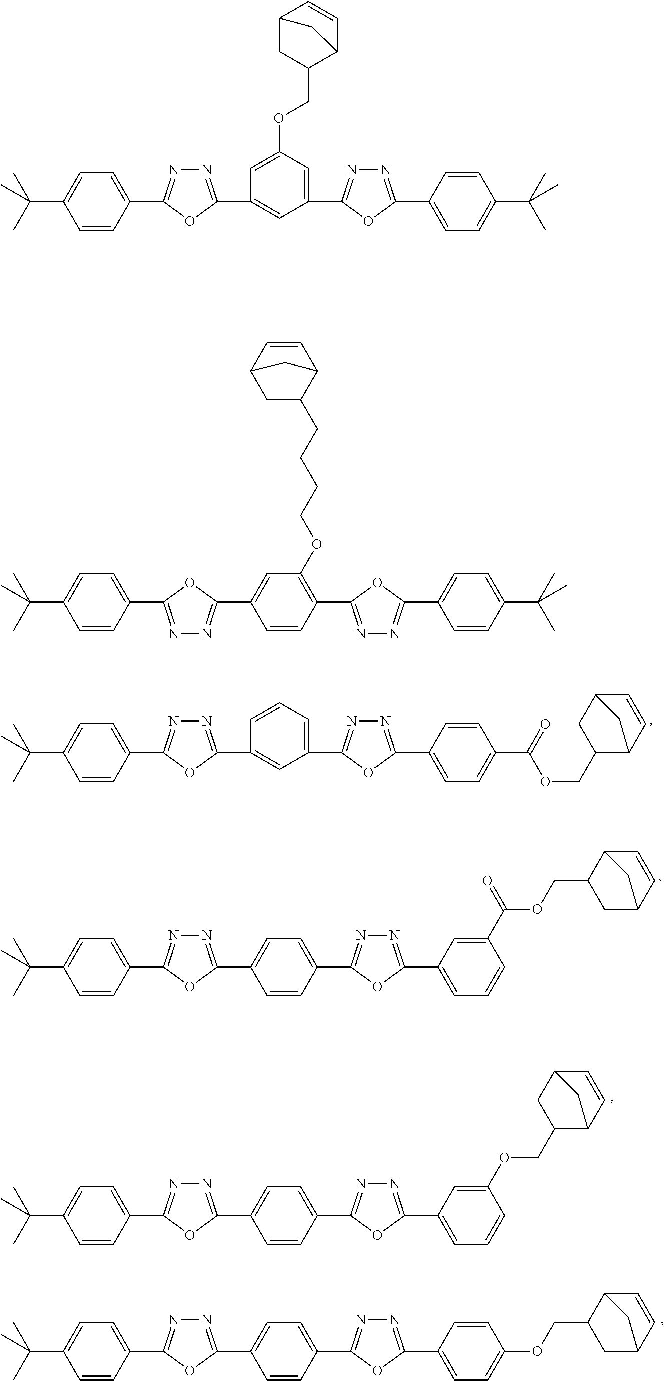

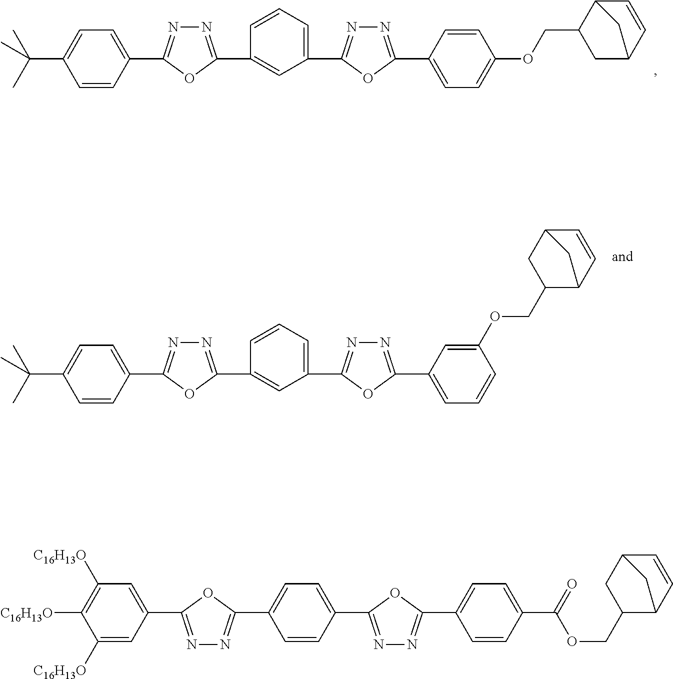

### The Polymeric Oxadizoles

In a second aspect, the invention relates to a compound represented by formula (II):

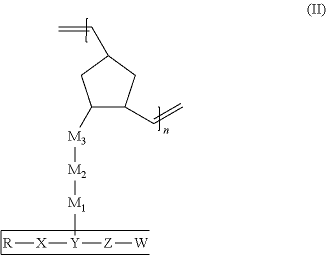

wherein:

R and W are each aryl and are unsubstituted, or substituted with substituents independently selected from the group consisting of different aryl groups, alkyl groups, halogens, fluoroalkyl groups, alkoxy groups, and amino groups; X and Z are each oxadiazole;

Y is absent or arene diyl;

wherein R—X—Y—Z—W taken together is a unit that is linked to the norbornene polymer by a linkage M₁-M₂-M₃, and wherein the linkage is attached to one of Y or W;

M₁ and M₃ are independently absent or represent

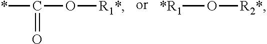

and is attached to the R—X—Y—Z—W unit through the carbon or oxygen atom on the ester, or through the ether oxygen atom, and M₂ is R₃;

R₁ and R₂ are independently absent or selected from the group consisting of alkane diyl, alkene diyl, alkyne diyl, and are nediyl, each of which are straight chain, branched chain or cyclic, having a carbon chain length of from 1 to 20 carbon atoms;

R₃ is absent or represents alkane diyl, alkene diyl, alkyne diyl, or arene diyl, each of which are straight chain, branched chain or cyclic, having a carbon chain length of from 1 to 20 carbon atoms; and n is an integer from about 1 to about 2,000.

For example, the polymers can be represented by formulas IIa, IIb, IIc, IId, IIe and IIf:

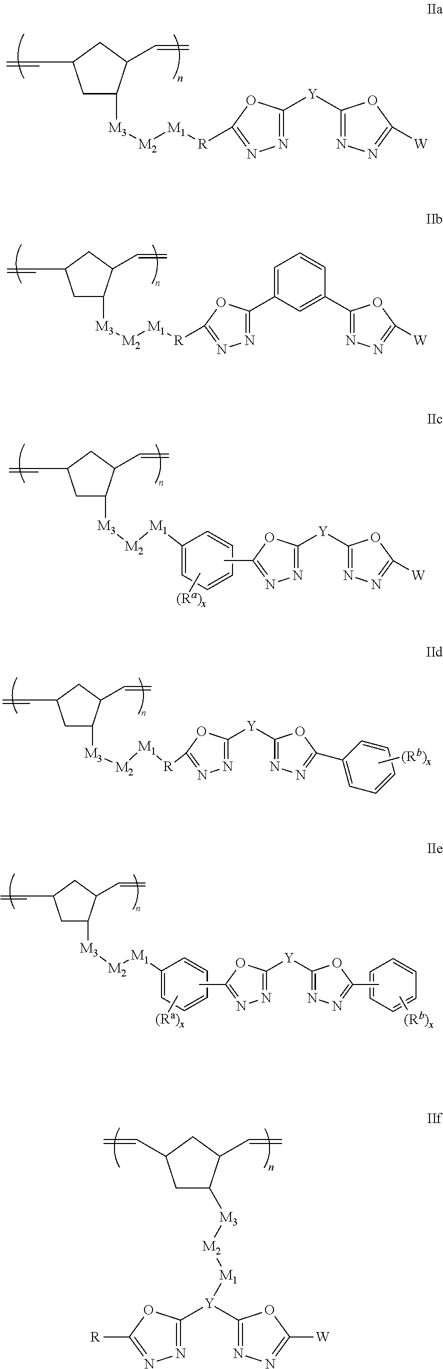

Where R, X, W, Y, Z, R^{a}, R^{b}, M₁, Mₛ, M₃, and x, are as described with respect to monomeric precursors. In polymer II, and IIa-IIf, n can be an integer from about 5 to about 2000. The subscript “n” refers to the number of repeat units in the polymer. More preferably, “n” is from about 700 to about 1,500 repeat units. Most preferably, “n” is from about 20 to about 500 repeat units.

This novel invention also provides a wide variety of functionalized amorphous polymers that are suitable incorporating high loadings of oxadiazoles while minimizing interaction between functional groups.

In a related embodiment, the invention relates to the following novel homo-polymers:

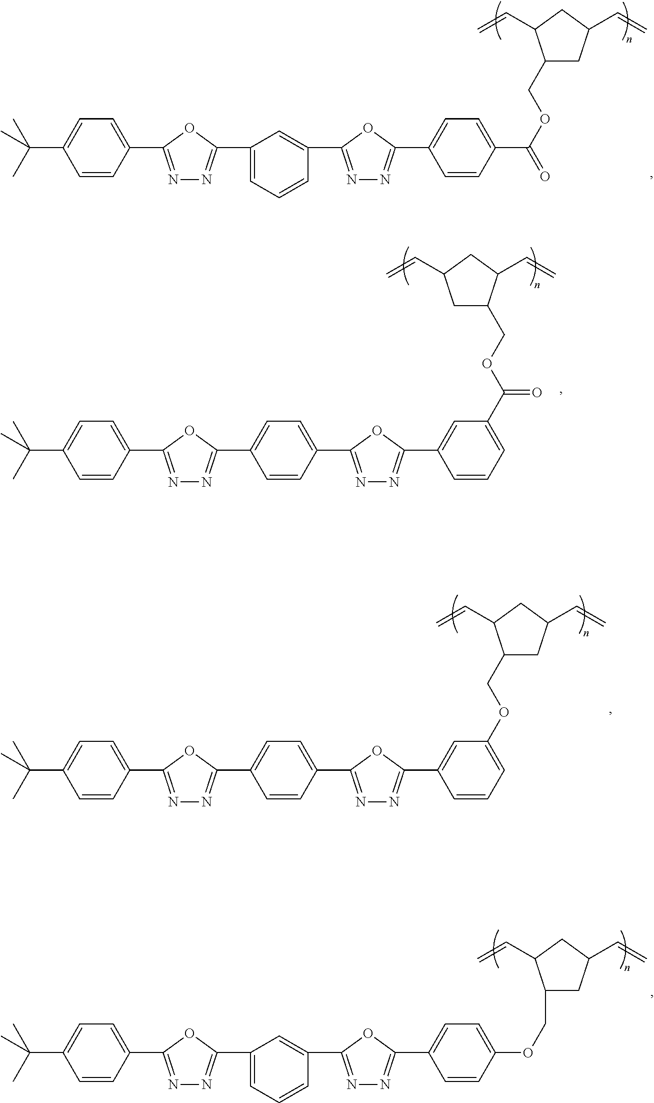

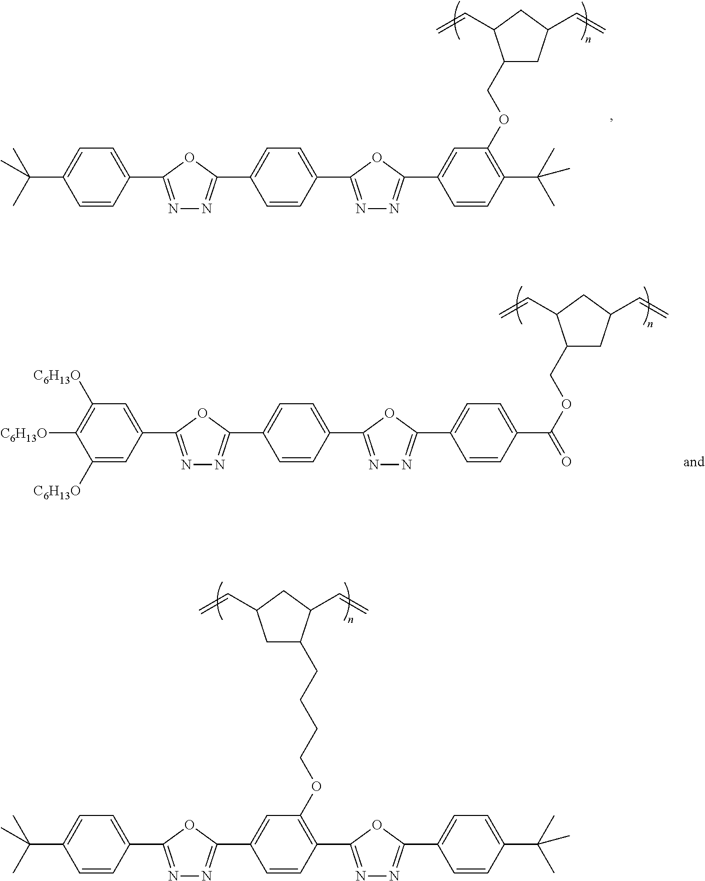

A related embodiment of the invention entails processes for preparing a polymer or copolymer where one or m ore monomeric compounds, I and Ia-If, is mixed with a ring opening metathesis catalyst and optionally one or more additional norbornenyl monomers, and then polymerized to form polynorbornenes II, and IIa-IIf or copolymers containing the repeat units illustrated in formulas Ia-If or I.

In another related embodiment, the invention relates to the polymer or copolymer product produced by polymerizing or copolymerizing a mixture containing at least one of monomers I, and Ia-If and optionally other suitable monomers in the presence of a ring opening metathesis catalyst.

In another related embodiment the polymerization process can be carried out by mixing another optional monomer into the monomeric mixture and then copolymerizing the mixture with a suitable ROMP catalyst to form a carbazole functionalized poly(norborne).

Poly(norbornene)s can be polymerized via ring-opening metathesis polymerization (ROMP), a living polymerization method resulting in polymers with controlled molecular weights, low polydispersities, and also allows for the easy formation of block co-polymers. See, for example, Fürstner, A. Angew. Chem., Int. Ed. 2000, 39, 3013; T. M. Trnka, T. M.; Grubbs, R. H. Acc. Chem. Res. 2001, 34, 18; Olefin Metathesis and Metathesis Polymerization, 2nd Ed.; Ivin, J., Mol, I. C., Eds.; Academic: New York, 1996; and Handbook of Metathesis, Vol. 3-Application in Polymer Synthesis; Grubbs, R. H., Ed.; Wiley-VCH: Weinheim, 2003, each of which is respectively incorporated herein by reference for the teachings regarding methods and catalysts for ROMP polymerizations. Catalysts commonly used by those skilled in the art include Grubb's ruthenium catalysts (below).

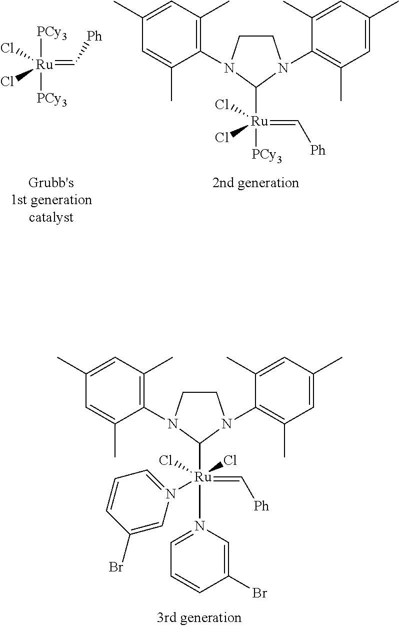

ROMP polymerizations can also be carried out with molybdenum or tun gsten catalysts such as those described by Schrock (Olefin Metathesis and Metathesis Polymerization, 2nd Ed.; Ivin, J., Mol, I. C., Eds.; Academic: New York which is respectively incorporated herein by reference for the teachings regarding molybdenum or tungsten catalysts for ROMP polymerizations). Furthermore, ruthenium-based ROMP initiators are highly functional-group tolerant, allowing for the polymerization of norbornene monomers containing fluorescent and phosphorescent metal complexes.

The copolymers disclosed herein can include copolymerized subunits derived from optionally substituted strained ring olefins such as, but not limited to, dicyclopentadienyl, norbornenyl, cyclooctenyl and cyclobutenyl monomers. Such monomers can be copolymerized with the compounds of formulas I, and Ia-If via ring opening metathesis polymerization using an appropriate metal catalyst, as would be obvious to those skilled in the art.

Further, the inventions can include, but is not limited to, (—CH₂)ₓSiCl₃, (—CH₂)ₓSi(OCH₂CH₃)₃, or (—CH₂)ₓSi(OCH₃)₃ dopants or substituents, where the monomers can be reacted with water under conditions known to those skilled in the art to form either thin film or monolithic organically modified sol-gel glasses, or modified silicated surfaces, where ₓ is an integer number from 0 to 25 (e.g., 0, 1, 2, 3, 4, 5, 6, 7, 8, 9, 10, 11, 12, 13, 14, 15, 16, 17, 18, 19, 20, 21, 22, 23, 24, and 25).

A related embodiment of the inventions relate to organic electronic devices containing a bis-oxadiazole material comprising one or more compounds of formula I, Ia-If, IIa-IIf, or II and blends thereof. Organic electronic devices include but are not limited to, active electronic components, passive electronic components, electroluminescent (EL) devices (e.g., organic light emitting devices (OLEDs)), photovoltaic cells, light-emitting diodes, field-effect transistors, phototransistors, radio-frequency ID tags, semiconductor devices, photoconductive diodes, metal-semiconductor junctions (e.g., Schottky barrier diodes), p-n junction diodes, p-n-p-n switching devices, photodetectors, optical sensors, phototransducers, bipolar junction transistors (BJTs), heterojunction bipolar transistors, switching transistors, charge-transfer devices, thin-film transistors, organic radiation detectors, infra-red emitters, tunable microcavities for variable output wavelength, telecommunications devices and applications, optical computing devices, optical memory devices, chemical detectors, combinations thereof, and the like.

A related embodiment of the inventions relate to an electron injecting/transporting and/or hole blocking layers, electron transport emissive materials, and host materials for an organic luminescence layer comprising formula (I) or (II). Compounds I, Ia-If, II, and IIa-IIf can each be used as a electron injecting/transporting and/or hole blocking component of organic electronic devices.

Charge-transport molecular and polymeric materials are semiconducting materials in which charges can migrate under the influence of an electric field. These charges may be present due to doping with oxidizing or reducing agents, so that some fraction of the transport molecules or polymer repeat units is present as radical cations or anions. More usually, charges are introduced b y injection from another material under the influence of an electric field. Charge-transport materials may be classified into hole- and electron-transport materials. In a hole-transport material, electrons are removed, either by doping or injection, from a filled manifold of orbitals to give positively charged molecules or polymer repeat units. Transport takes place by electron-transfer between a molecule or polymer repeat unit and the corresponding radical cation; this can be regarded as movement of a positive charge (hole) in the opposite direction to this electronic motion. In an electron-transport material, extra electrons are added, either by doping or injection; here the transport process includes electron-transfer from the radical anion of a molecule or polymer repeat unit to the corresponding neutral species.

The organic electronic devices described herein can contain the following layers: a transparent substrate, a transparent conducting anode overlying the substrate, a hole transport layer and/or an electron blocking layer over the anode, a light-emitting layer, an electron transport and/or hole-blocking layer, and a cathode layer.

A plurality of layers of charge-transport material can be produced to form a charge-transport layer that can have a thickness of about 0.01 to 1000 μm, 0.05 to 100 μm, 0.05 to 10 μm. The length and width of the charge-transport layer can vary depending on the application, but in general, the length can be about 0.01 μm to 1000 cm, and the width can be about 0.01 μm to 1000 cm.

It should also be noted that the charge-transport materials could be used as mixtures with other electron transport materials including those described herein, as well as others. Likewise the charge-transport materials could be used in combination with other hole transport materials, sensitizers, emitters, chromophores, and the like, to add other functionality to devices.

A related embodiment of the inventions relate to a composition of matter for an electron injecting/transporting and/or hole blocking layers, electron transport emissive materials, and host materials for an organic luminescence layer comprising formulas (I) or (II) in combination with a phosphorescent dopant. In related embodiments of the devices of the inventions, the light-emitting layer of the device can comprise a poly(norbornene) monomer, homopolymer, or copolymer compound that can be represented by polymer II, IIa-IIf and monomers I, Ia-If. In some aspects, the emitting layer of the invention can be formed using the mixture of oxadiazole polymer host and a guest emitter. The guest emitter could be one or more phosphorescent metal complexes further described below.

The norbornene monomers, polymers and copolymers of the present inventions can be doped with phosphorescent metal complexes as guests or co-polymerized with metal phosphorescent complexes containing a polymerizable norbornenyl group. The phosphorescent dopant is preferably a metal complex comprising at least one metal selected from the group consisting of Ir, R d, Pd, Pt, Os and Re, and the like. More specific examples of the phosphorescent dopants include but are not limited to metal complexes such as tris(2-phenylpyridinato-N,C²)ruthenium, bis(2-phenylpyridinato-N,C²)palladium, bis(2-phenylpyridinato-N,C²)platinum, tris(2-phenylpyridinato-N,C²)osmium, tris(2-phenylpyridinato-N,C²)rhenium, octaethyl platinum porphyrin, octaphenyl platinum porphyrin, octaethyl palladium porphyrin, octaphenyl palladium porphyrin, iridium(III)bis[(4,6-difluorophenyl)-pyridinato-N,C^{2′}]picolinate (Firpic), tris-(2-phenylpyridinato-N,C²)iridium Ir(ppy)₃), green material bis-(2-phenylpyridinato-N,C²)iridium(acetylacetonate) (Ir(ppy)₂ (acac), and red material 2,3,7,8,12,13,17,18-octaethyl-21H, 23H-porphine platinum(II) (PtOEP) as w ell as other known to those skilled in the art of OLEDs and metallo-organic chemistry. In one preferred embodiment, the guest emitter is Ir(ppy)₃.

Preferably the organic electroluminescence device emits red light, yellow light, green light, blue light, white light or light with a broad band containing multiple color peaks. The norbornene compounds of the present invention can also be doped with other polymers to obtain white organic light-emitting diodes.

In a related embodiment, the invention relates to the following novel compounds, whose synthesis is described in the Examples below. These compounds are used in the Examples below as synthetic intermediates for attaching desired R—X—Y—Z—W groups to the norbornenyl/M₁/M₂/M₃ groups, in order prepare the monomers and polymers described herein:

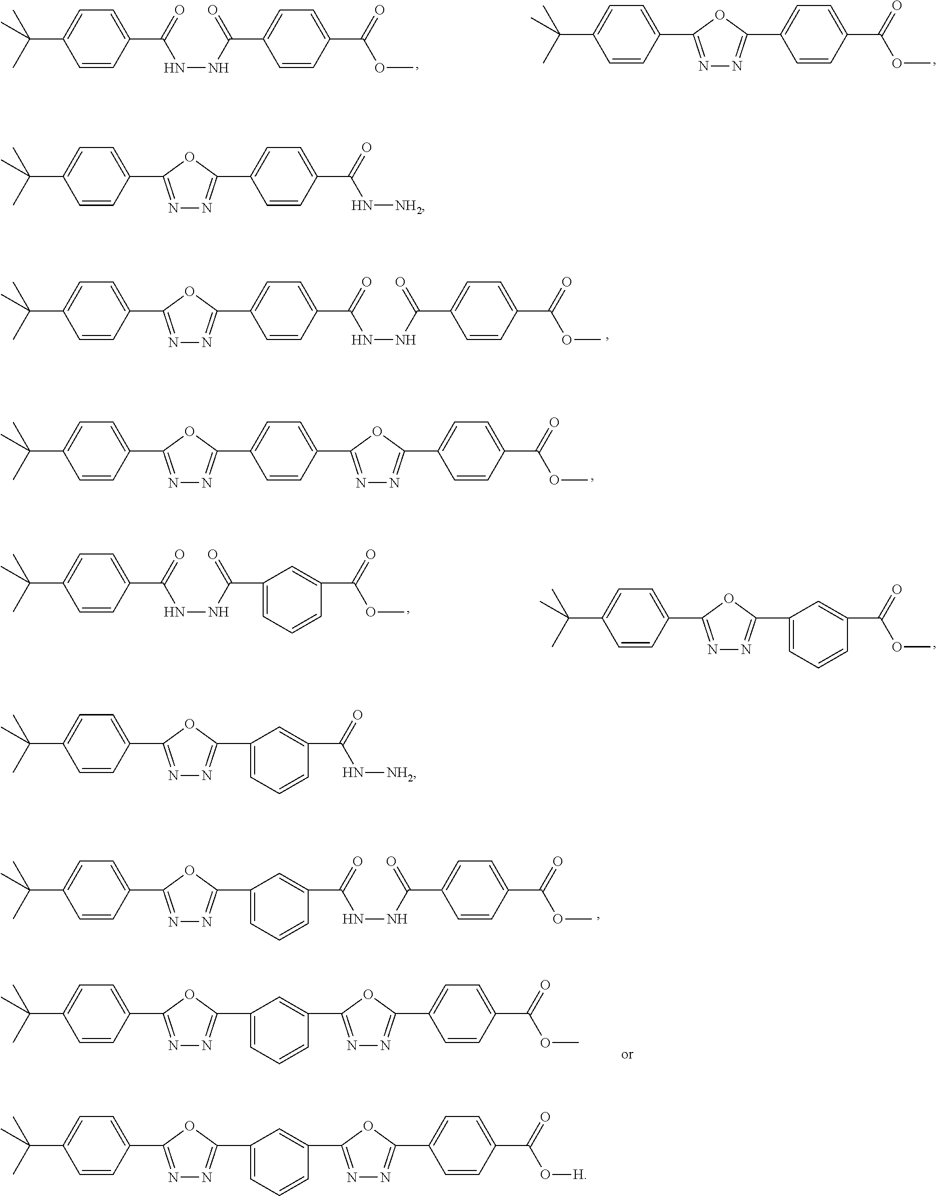

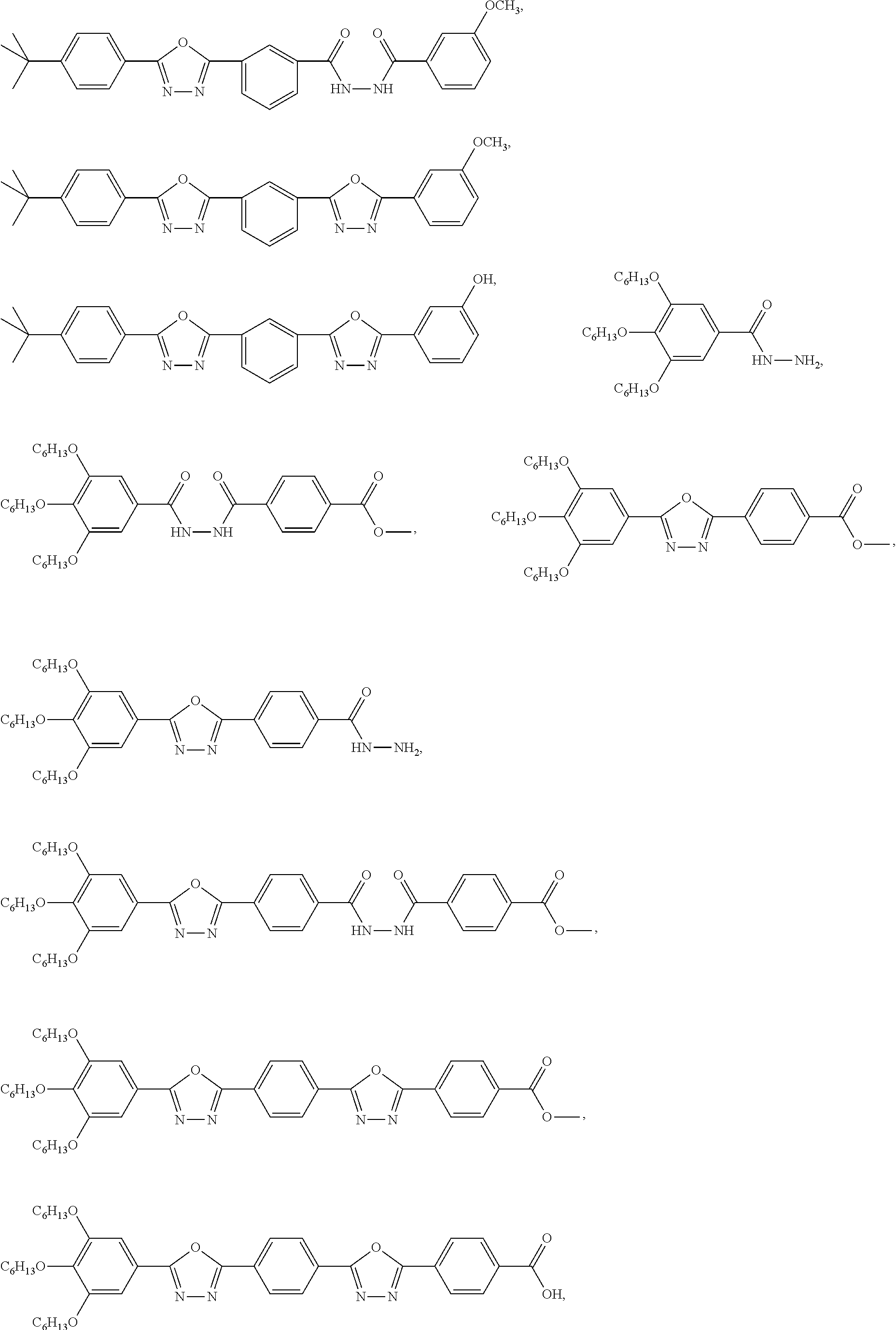

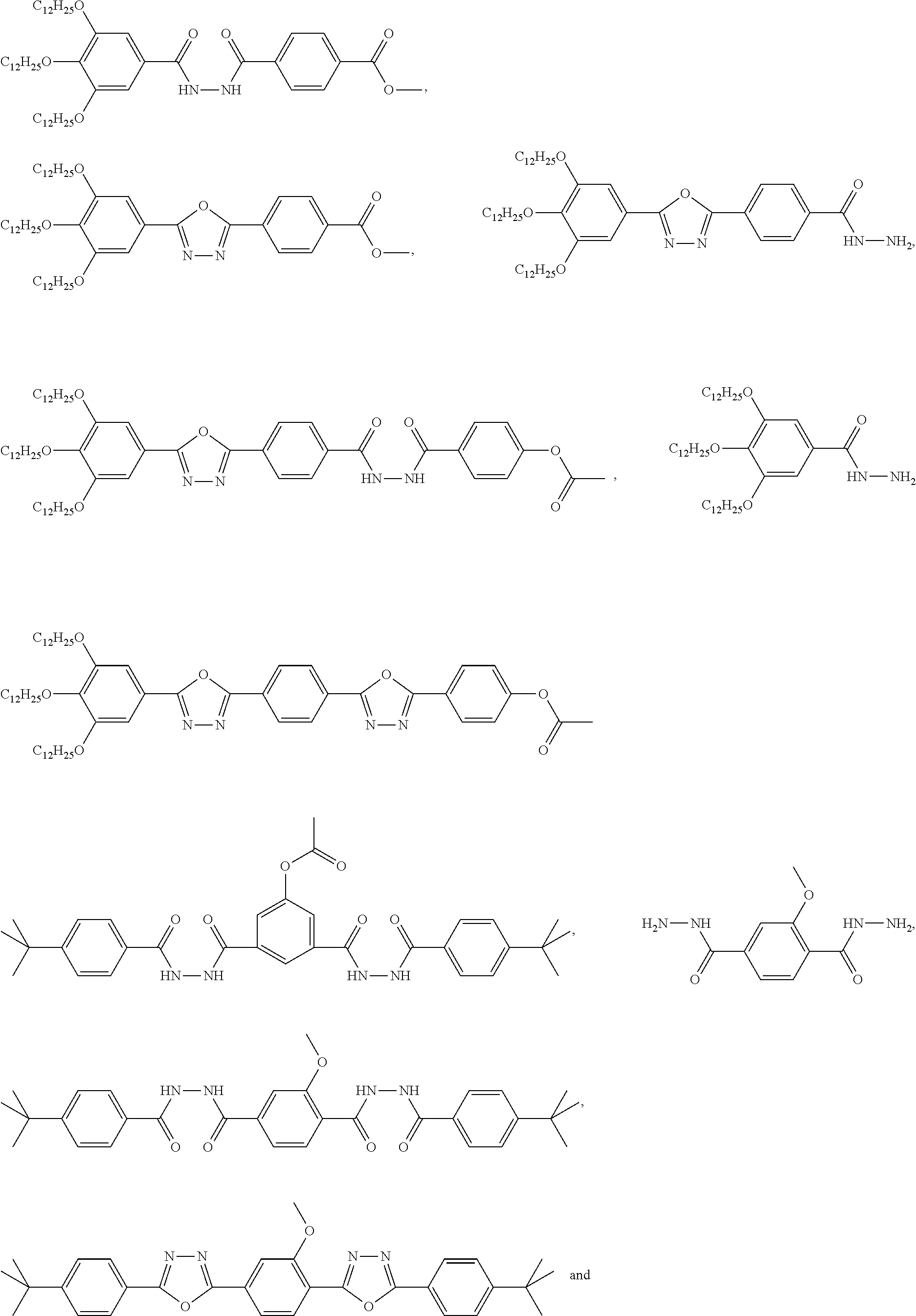

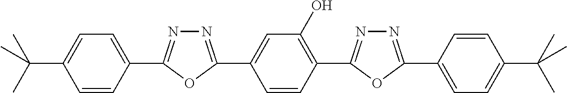

It would be obvious to one of ordinary skill in the art how to prepare many substituted variations of these same compounds by merely employing alternatively substituted aromatic starting materials in synthetic procedures analogous to those described in the Examples below.

### Experimental

The following examples are put forth to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g. amounts, temperature, etc.) but some errors and deviations should be accounted for. Unless otherwise indicated, parts are parts by weight, temperature is in ° C. or is at ambient temperature and pressure is at or near atmospheric.

### Preparation Example 1

### Synthesis of YZ-I-207

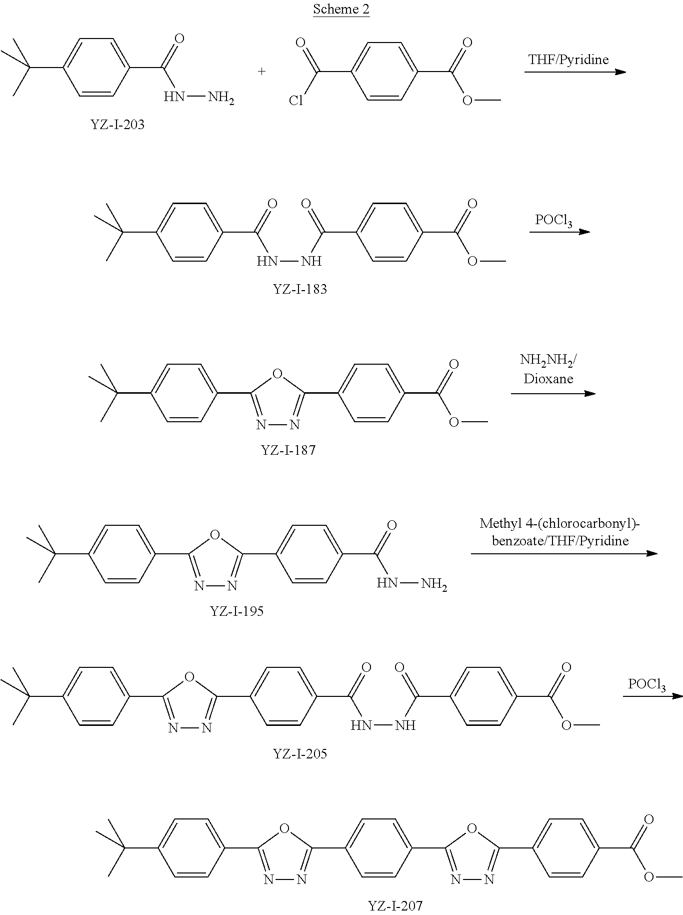

### Preparation of Starting Material:4-tert-Butylbenzhydrazine (YZ-I-203)

To methyl 4-tert-butylbenzoate (40.0 g, 0.21 mol) in dioxane (120 ml) was added hydrazine hydrate (60.0 g, 1.20 mol). The reaction mixture was refluxed for 28 hours. The reaction mixture was cooled down to room temperature and poured into water (1000.0 ml). The white product solid was collected by filtration and dried under vacuum. The yield of the reaction was 36.0 g (90.0%).

¹H NMR (400 MHz, CDCl₃) δ: 7.67 (d, 2H, J=8.4 Hz), 7.40 (d, 2H, J=8.4 Hz), 4.15 (br, 2H, NH₂), 1.29 (s, 9H, 3×CH₃) ppm. ¹³C NMR (100 MHz, CDCl₃) δ: 168.54, 155.38, 129.54, 126.72, 125.57, 34.90, 31.07 ppm.

### Step 1: Methyl 4-(2-(4-tert-butylbenzoyl)hydrazinecarbonyl)benzoate (YZ-I-183)

To a solution of 4-tert-butylbenzhydrazine (2.0 g, 10.04 mmol) in dry THF (60 ml) was slowly added methyl 4-(chlorocarbonyl)benzoate (2.1 g, 10.06 mmol) at room temperature under nitrogen. During addition of methyl 4-(chlorocarbonyl)benzoate, a white solid appeared. The reaction mixture was stirred for 4 hours at room temperature and then pyridine (5.0 ml) was added and stirred for an additional 30 minutes. The reaction mixture was poured into water (250 ml). The white solid was collected by filtration. After drying under vacuum, 3.2 g (86.5%) product was obtained as a white powder.

¹H NMR (400 MHz, DMSO-d₆) δ: 10.70 (s, 1H, NH), 10.51 (s, 1H, NH), 8.08 (d, 2H, J=8.0 Hz), 8.02 (d, 2H, J=8.0 Hz), 7.85 (d, 2H, J=8.0 Hz), 7.53 (d, 2H, J=8.0 Hz), 3.89 (s, 3H, OCH₃), 1.29 (s, 9H, 3×CH₃) ppm.

### Step 2: Methyl 4-(5-(4-tert-butylphenyl)-1,3,4-oxadiazol-2-yl)benzoate (YZ-I-187)

Methyl 4-(2-(4-tert-butylbenzoyl)hydrazinecarbonyl)benzoate (2.46 g, 6.94 mmol) was suspended in POCl₃ (20.0 ml) and heating was started. The reaction was kept at 85° C. During heating, the white solid starting materials dissolved into a clear solution and the reaction was monitored by thin layer chromatography. After 4 hours, the reaction mixture was brought to room temperature and was carefully dropped into ice-water (200 ml). The white solid precipitated out was collected by filtration and dried under vacuum to give 2.1 g (90.1%) of white powder.

¹H NMR (400 MHz, CDCl₃) δ: 8.22 (s, 2H), 8.21 (s, 2H), 8.08 (d, 2H, J=8.4 Hz), 7.56 (d, 2H, J=8.4 Hz), 3.98 (s, 3H, OCH₃), 1.38 (s, 9H, 3×CH₃) ppm. ¹³C NMR (100 MHz, CDCl₃) δ: 166.13, 165.16, 163.60, 155.73, 132.70, 130.25, 127.71, 126.90, 126.79, 126.13, 120.71, 52.48, 35.13, 31.09 ppm.

### Step 3: 4-(5-(4-tert-Butylphenyl)-1,3,4-oxadiazol-2-yl)benzohydrazine (YZ-I-195)

To a solution of methyl 4-(5-(4-tert-butylphenyl)-1,3,4-oxadiazol-2-yl)benzoate (2.38 g, 7.07 mmol) in dioxane (50.0 ml) was added hydrazine hydrate (7.0 ml). The reaction mixture was heated to 100° C. and kept at this temperature for 23 hours. The reaction mixture was cooled down to room temperature and poured into water (200.0 ml). The white solid was collected by filtration and dried under vacuum. The yield is 2.05 g (86.1%).

¹H NMR (400 MHz, DMSO-d₆) δ: 10.02 (s, br, 1H, NH), 8.18 (d, 2H, J=8.8 Hz), 8.06 (d, 2H, J=8.8 Hz), 8.03 (d, 2H, J=8.8 Hz), 7.64 (d, 2H, J=8.8 Hz), 4.65 (s, br, 2H, NH₂), 1.32 (s, 9H, 3×CH₃) ppm.

### Step 4: Methyl 4-(2-(4-(5-(4-tert-butylphenyl)-1,3,4-oxadiazol-2yl)benzoyl)hydrazinecarbonyl)-benzoate (YZ-I-205)

To a solution of 4-(5-(4-tert-butylphenyl)-1,3,4-oxadiazol-2-yl)benzohydrazine (2.0 g, 5.95 mmol) in THF (80.0 ml) was slowly added methyl 4-(chlorocarbonyl)benzoate (1.3 g, 6.55 mmol) at room temperature under nitrogen. The reaction mixture was stirred for 22 hours at room temperature and then pyridine (15.0 ml) was added. The reaction mixture was stirred an additional half an hour and then two-thirds of the solvent was removed, after which water (200.0 ml) was added. The white precipitate formed was collected by filtration, washed with water and dried under vacuum gave 2.64 g (89.2%) white solid.

¹H NMR (400 MHz, DMSO-d₆) δ: 10.85 (s, br, 1H, NH), 10.83 (s, br, 1H, NH), 8.28 (d, 2H, J=8.4 Hz), 8.16-8.04 (m, 8H), 7.65 (d, 2H, J=8.4 Hz), 3.89 (s, 3H, OCH₃), 1.32 (s, 9H, 3×CH₃) ppm.

### Step 5: Methyl 4-(5-(4-(5-(4-tert-Butylphenyl)-1,3,4-oxadiazol-2-yl)phenyl)-1,3,4-oxadiazol-2-yl)-benzoate (YZ-I-207)

Methyl 4-(2-(4-(5-(4-tert-butylphenyl)-1,3,4-oxadiazol-2-yl)benzoyl)-hydrazinecarbonyl)benzoate (2.5 g, 5.05 mmol) and POCl₃ (50 ml) were taken into a 100 mL round bottom flask. The reaction was kept at 100° C. During the heating (about 30 min) the starting material solid disappeared. After 2 hours of the reaction, the solid appeared due to the insolubility of product in POCl₃. After 7 hours at 100° C., the reaction mixture was allowed to cool down to room temperature and was slowly dropped into ice-water (200.0 ml). The white solid formed was collected by filtration, dried under vacuum and gave 2.2 g (91.7%) in yield. The purification and characterization were difficult due to the very low solubility of this compound in common organic solvents.

### Preparative Example 2

### Synthesis of YZ-I-259

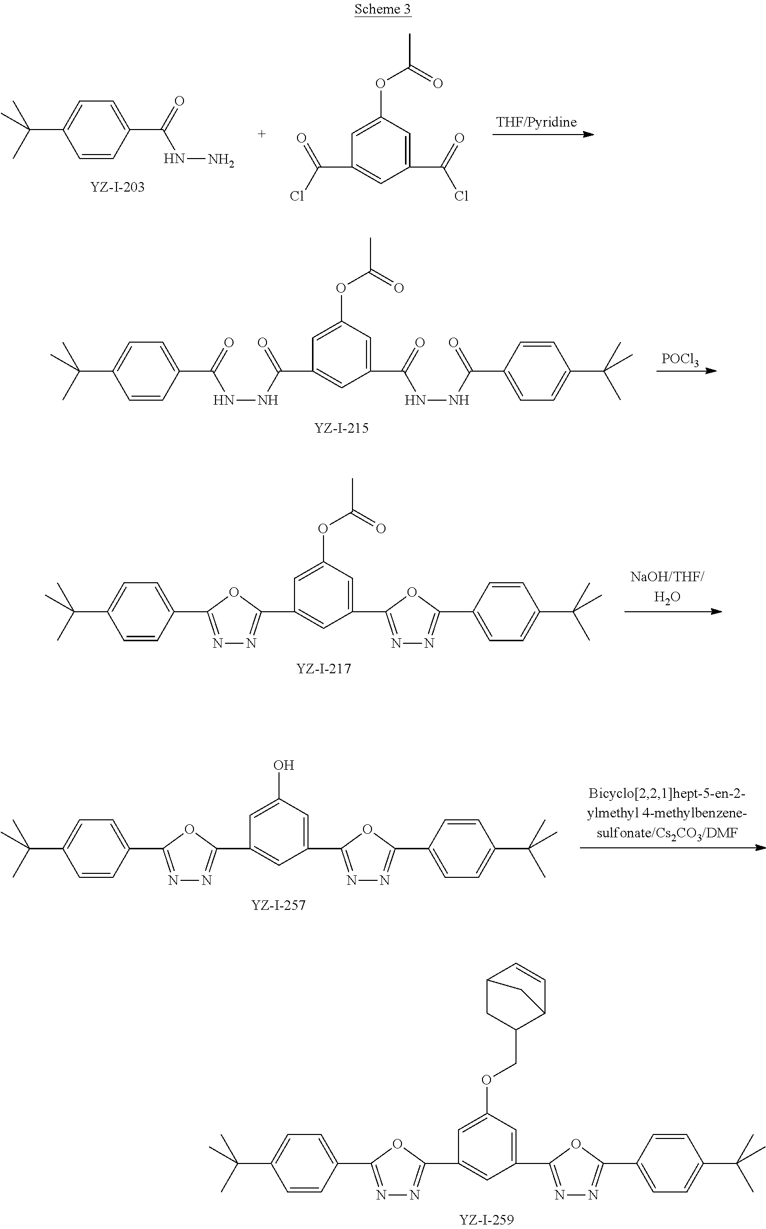

### Step 1: 3,5-Bis(2-(4-tert-butylbenzoyl)hydrazinecarbonyl)phenyl acetate (YZ-I-215)

4-tert-Butylbenzohydrazine (3.2 g, 16.64 mmol) (YZ-I-203) and 3,5-bis(chlorocarbonyl)phenyl acetate (2.2 g, 8.43 mmol) were taken into dry tetrahydrofuran (50.0 ml) at room temperature under nitrogen. The reaction mixture was stirred at room temperature for 6 hours and then pyridine (8.0 ml) was added and stirred for another 1 hour. Water (200.0 ml) was added into the reaction mixture. The brown solid was collected by filtration and dried under vacuum and gave 4.6 g (95.8%) yield .

¹H NMR (400 MHz, CDCl₃) δ: 10.37 (s, br, 2H, 2×NH), 9.83 (s, br, 2H, 2×NH), 8.11 (s, 1H), 7.71 (d, 4H, J=8.4 Hz), 7.54 (s, 2H), 7.25 (d, 4H, J=8.4 Hz), 2.11 (s, 3H, CH₃) 1.24 (s, 18H, 6×CH₃) ppm.

### Step 2: 3,5-Bis(5-(4-tert-butylphenyl)-1,3,4-oxadiazol-2-yl)phenyl acetate (YZ-I-217)

3,5-Bis(2-(4-tert-butylbenzoyl)hydrazinecarbonyl)phenyl acetate (2.1 g, 3.67 mmol) was added in POCl₃ (20.0 ml). The reaction was heated to 100° C. and kept at this temperature for 2 hours. After cooling dow n to room temperature, the reaction mixture was slowly dropped into ice-water (300.0 ml). The brown solid formed was collected by vacuum filtration. The crude product was dried and purified by silica gel column using dichloromethane/ethyl acetate (9.5:0.5) as eluent. After removal of the solvents, a pure white solid product was obtained in 0.58 g (29.4%) yield after recrystallization from dichloromethane/methanol.

¹H NMR (400 MHz, CDCl₃) δ: 8.75 (t, 1H, J=1.2 Hz), 8.11 (d, 4H, J=8.4 Hz), 8.07 (d, 2H, J=1.2 Hz), 7.58 (d, 4H, J=8.4 Hz), 2.42 (s, 3H, CH₃), 1.39 (s, 18H, 6×CH₃) ppm. ¹³C NMR (100 MHz, CDCl₃) δ: 168.81, 165.30, 162.74, 155.81, 151.57, 126.98, 126.45, 126.15, 122.99, 122.16, 120.59, 35.14, 31.09, 21.04 ppm. MS-EI (m/z): [M]⁺ calcd for C₃₂H₃₂N₄O₄ 536.2, found 536.2.

### Step 3: 3,5-Bis(5-(4-tert-butylphenyl)-1,3,4-oxadiazol-2-yl)phenol (YZ-I-257)

3,5-Bis(5-(4-tert-butylphenyl)-1,3,4-oxadiazol-2-yl)phenyl acetate (1.2 g, 2.24 mmol) and NaOH (0.2 g, 5.00 mmol in 1.0 ml of water) were taken into THF (40.0 ml). The reaction was heated to reflux and kept at reflux for 30 minutes. During heating the reaction solution was changed to yellow. After cooling down to room temperature, concentrated HCl (3.0 ml) was added into the reaction mixture. The yellow color observed disappeared and a white solid appeared. After removal of the reaction solvents, water (100.0 ml) was added. The white solid product was collected by filtration. After drying under vacuum, the product as a white solid was obtained in 1.07 g (96.4%) yield.

¹H NMR (400 MHz, DMSO-d₆) δ: 8.18 (t, 1H, J=1.6 Hz), 8.07 (d, 4H, J=8.8 Hz), 7.70 (d, 2H, J=1.6 Hz), 7.65 (d, 4H, J=8.8 Hz), 1.33 (s, 18H, 6×CH₃) ppm.

### Step 4: 5,5′-(5-(Bicyclo[2,21]hept-5-en-2-ylmethoxy)-1,3-phenylene)bis(2-(4-tert-buyylphenyl)-1,3,4-oxadiazole (YZ-I-259)

To a solution of 3,5-bis(5-(4-tert-butylphenyl)-1,3,4-oxadiazol-2-yl)phenol (1.0 g, 2.02 mmol) and bicyclo[2,2,1]hept-5-en-2-ylmethyl 4-methylbenzenesulfonate (1.6 g, 5.75 mmol) in DMF (25.0 ml) was added Cs₂CO₃ (4.0 g, 12.28 mmol) at room temperature under nitrogen. The reaction was carried out at 100° C. for 3 hours After cooling down to room temperature, water (120.0 ml) was added into the reaction mixture. A brown solid precipitate w as collected by filtration and washed with methanol and then dried under vacuum. The crude product was purified by silica gel column using dichloromethane/ethyl acetate (9.5: 0.5) as the eluent. After the removal of the solvents, a pure white solid product was obtained in 0.84 g (69.4%) yield after recrystallization from dichloromethane/methanol.

¹H NMR (400 MHz, CDCl₃) δ: 8.42 and 8.40 (two t, 1H, J=1.2 Hz, endo and exo), 8.11 (d, 4H, J=8.4 Hz), 7.86 and 7.82 (two d, 2H, J=1.2 Hz, endo and ex o), 7.57 (d, 4H, J=8.4 Hz), 6.24-6.00 (m, 2H, C═C—H, endo and exo), 4.24-3.72 (m, 2H, OCH₂, endo and exo), 3.12 (s, br), 2.91 (m, br), 2.63 (m, br), 1.98 (m), 1.52 (m), 1.39 (s, 18H, 6×CH₃), 1.40-1.23 (m), 0.71 (m) ppm. ¹³C NMR (100 MHz, CDCl₃) δ: 165.10, 163.45, 159.94, 155.64, 137.82, 136.97, 136.28, 132.20, 126.91, 126.10, 126.05, 120.73, 117.08, 117.00, 115.68, 73.03, 72.25, 49.43, 45.08, 43.87, 43.69, 42.23, 41.61, 38.49, 38.26, 35.10, 31.08, 29.61, 28.96 ppm. MS (m/z): [M+1]⁺ calcd for C₃₄H₃₂N₄O₃ 601.3, found 601.3.

### Preparative Example 3

### Synthesis of YZ-I-273

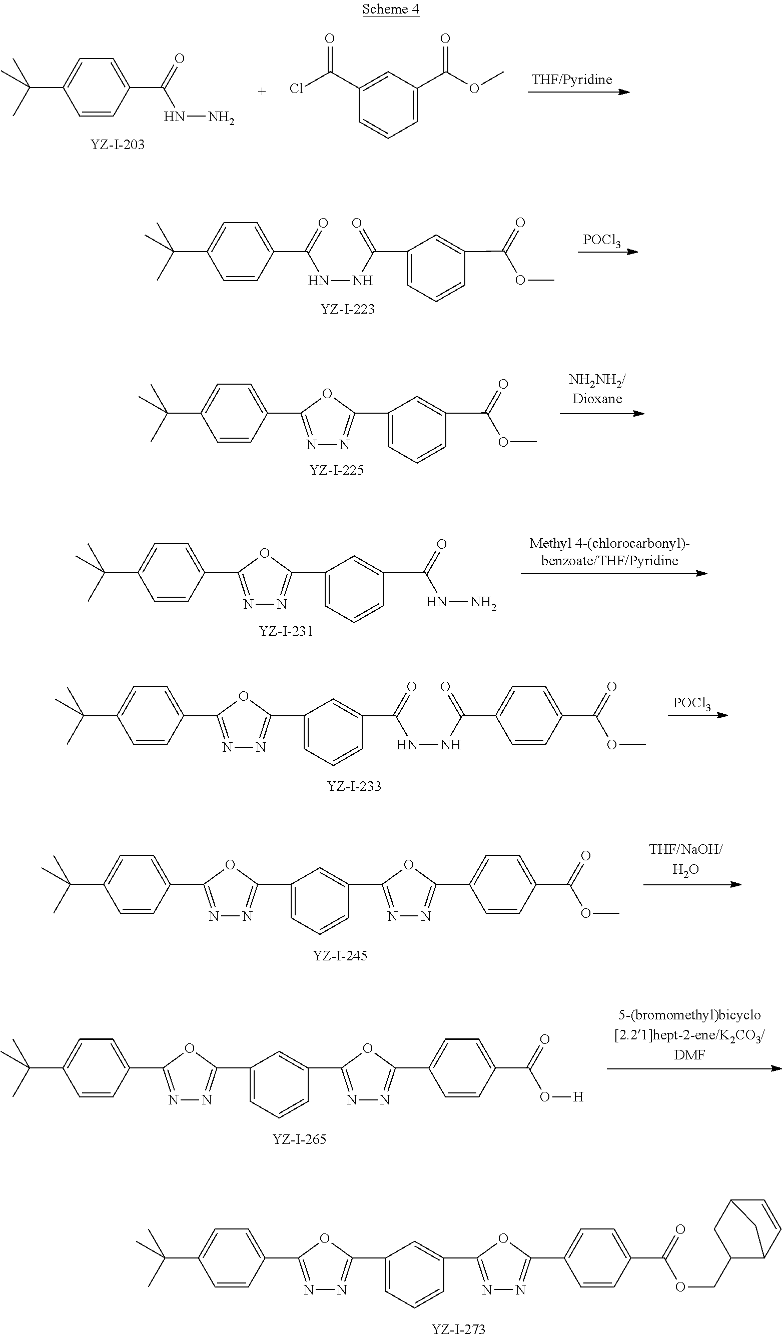

### Step 1: Methyl 3-(2-(4-tert-butylbenzoyl)hydrazinecarbonyl)benzoate (YZ-I-223)

To a solution of 4-tert-butylbenzohydrazine (5.8 g, 30.17 mmol) in dry tetrahydrofuran (100.0 ml) was slowly added methyl 3-(chlorocarbonyl)benzoate (6.0 g, 30.21 mmol) at room temperature under nitrogen. During the addition of methyl 3-(chlorocarbonyl)benzoate, the white solid appeared. The reaction mixture was stirred for 15 hours and then pyridine (15.0 ml) was added and stirred for another 1 hour. The reaction mixture was poured into water (300.0 ml). The white solid was collected by filtration, and dried overnight under vacuum and gave 10.0 g (93.4%) in yield.

¹H NMR (400 MHz, DMSO-d₆) δ: 10.73 (s, 1H, NH), 10.50 (s, 1 h, NH), 8.52 (t, 1H, J=1.6 Hz), 8.17 (tt, 2H, J₁=7.2 Hz, J₂=1.6 Hz), 7.86 (d, 2H, J=8.4 Hz), 7.69 (t, 1H, J=7.2 Hz), 7.54 (d, 2H, J=8.4 Hz), 3.90 (s, 3H, OCH₃), 1.31 (s, 9H, 3×CH₃) ppm.

### Step 2: Methyl 3-(5-(4-tert-butylphenyl)-1,3,4-oxadiazol-2-yl)benzoate (YZ-I-225)

Methyl 3-(2-(4-tert-butylbenzoyl)hydrazinecarbonyl)benzoate (9.5 g, 26.81 mmol) was suspended in POCl₃ (50.0 ml). The reaction was heated to 90° C. and kept at this temperature for 2 hours. After cooling down to room temperature, the reaction mixture was slowly dropped into ice-water (300.0 ml). The brown color solid formed was collected by vacuum filtration. The crude product was dried and purified by silica gel column using dichloromethane/ethyl acetate (9.5:0.5) as the eluent. After the removal of the solvents, a pure white solid product was obtained in 7.4 g (82.2%) yield by recrystallization from acetone/water. ¹H NMR (400 MHz, CDCl₃) δ: 8.77 (t, 1H, J=1.2 Hz), 8.36 (dt, 1H, J₁=7.6 Hz, J₂=1.2 Hz), 8.22 (dt, 1H, J₁=7.6 Hz, J₂=1.2 Hz), 8.09 (d, 2H, J=8.8 Hz), 7.64 (t, 1H, J=7.6 Hz). 7.56 (d, 2H, J=8.8 Hz), 4.00 (s, 3H, OCH₃), 1.38 (s, 9H, 3×CH₃) ppm. ¹³C NMR (100 MHz, CDCl₃) δ: 166.05, 164.97, 163.56, 155.54, 132.43, 131.17, 131.00, 129.30, 127.82, 126.84, 126.07, 124.44, 120.82, 52.48, 35.09. 31.08 ppm.

### Step 3: 3-(5-(4-tert-butylphenyl)-1,3,4-oxadiazol-2-yl)benzohydrazine (YZ-I-231)

To a solution of methyl 3-(5-(4-tert-butylphenyl)-1,3,4-oxadiazol-2-yl)benzoate (7.0 g, 20.81 mmol) in dioxane (125.0 ml) and ethanol (25.0 ml) was added hydrazine hydrate (25.0 ml). The reaction mixture was heated to 100° C. and kept at this temperature for 7 hours. The reaction mixture was cooled down to room temperature. Water (300.0 ml) was then added to reaction mixture. The white product solid was collected by filtration and dried under vacuum. The yield was 7.0 g (100%).

¹H NMR (400 MHz, DMSO-d₆) δ: 10.07 (s, br, 1H, NH), 8.55 (t, 1H, J=1.6 Hz), 8.25 (dt, 1H, J₁=8.0 Hz, J₂=1.6 Hz), 8.06 (d, 2H, J=8.8 Hz), 7.69 (t, 1H, J=8.0 Hz), 7.65 (d, 2H, J=8.8 Hz), 4.60 (s, br, 2H, NH₂), 1.33 (s, 9H, 3×CH₃) ppm.

### Step 4: Methyl 4-(2-(3-(5-(4-tert-butylphenyl)-1,3,4-oxadiazol-2-yl)benzoyl)hydrazinecarbon (YZ-233)

To a solution of 3-(5-(4-tert-butylphenyl)-1,3,4-oxadiazol-2-yl)benzo-hydrazine (2.0 g, 5.95 mmol) in dry tetrahydrofuran (80.0 ml) and DMF (5.0 ml) was slowly added methyl 4-(chlorocarbonyl)benzoate (1.3 g , 6.55 mmol) at room temperature under nitrogen. During the addition of methyl 3-(chlorocarbonyl)benzoate, a white solid appeared. The reaction mixture was stirred at room temperature for 21 hours and then pyridine (10.0 ml) was added and stirred for another 1 hour. The reaction mixture was poured into water (300.0 ml). The white solid was collected by filtration and dried overnight under vacuum gives in 2.8 g (94.3%) yield.

¹H NMR (400 MHz, DMSO-d₆) δ: 10.85 (s, br, 1H, 2×NH), 8.66 (s, 1H), 8.34 (d, 1H, J=8.0 Hz), 8.17 (d, 1H, J=8.0 Hz), 8.10-8.00 (m, 6H), 7.81 (t, 1H, J=8.0 Hz), 7.66 (d, 2H, J=8.4 Hz), 3.89 (s, 3H, OCH₃), 1.33 (s, 9H, 3×CH₃) ppm.

### Step 5: Methyl 4-(5-(3-(5-(4-tert-butylphenyl)-1,3,4-oxadiazol-2-yl)phenyl)-1,3,4-oxadiazol-2-yl)benzoate (YZ-I-245)

Methyl 4-(2-(3-(5-(4-tert-butylphenyl)-1,3,4-oxadiazol-2-yl)benzoyl)-hydrazinecarbonyl)benzoate (2.4 g, 4.81 mmol) was added in POCl₃ (30.0 ml). The reaction was heated to 90° C. and kept at this temperature for 7.5 hours. After cooling down to room temperature, the reaction mixture was slowly dropped into ice-water (300.0 ml). The white solid formed was collected by vacuum filtration. The crude product was dried and purified by silica gel column using dichloromethane/ethyl acetate (9:1) as the eluent. After the removal of solvents, a pure white solid product was obtained in 1.47 g (63.6%) yield by recrystallization from dichloromethane/methanol.

¹H NMR (400 MHz, CDCl₃) δ: 8.89 (t, 1H, J=1.2 Hz), 8.37 (dd, 2H, J₁=8.0 Hz, J₂=1.2 Hz), 8.27 (d, 2H, J=8.8 Hz), 8.24 (d, 2H, J=8.8 Hz), 8.11 (d, 2H, J=8.8 Hz), 7.76 (t, 1H, J=8.0 Hz), 7.59 (d, 2H, J=8.8 Hz), 3.99 (s, 3H, OCH₃), 1.39 (s, 9H, 3×CH₃) ppm. ¹³C NMR (100 MHz, CDCl₃) δ: 166.06, 165.18, 164.26, 163.30, 155.73, 133.04, 130.34, 130.09, 130.00, 129.78, 127.35, 127.01, 126.92, 126.15, 125.23, 125.06, 124.71, 120.70, 52.53, 35.13, 31.10 ppm. MS-EI (m/z): [M]⁺ calcd for C₂₈H₂₄N₄O₄ 480.2, found 480.2.

### Step 6: 4-(5-(3-(5-(4-tert-Butylphenyl)-1,3,4-oxadiazol-2-yl)phenyl)-1,3,4-oxadiazol-2-yl)benzoic acid (YZ-I-265)

Methyl 4-(5-(3-(5-(4-tert-butylphenyl)-1,3,4-oxadiazol-2-yl)phenyl)-1,3,4-oxadiazol-2-yl)benzoate (1.2 g, 2.50 mmol) was taken into THF (150.0 ml) and ethanol (30.0 ml). The reaction mixture was heated to reflux. When the starting material was dissolved in THF/ethanol, NaOH (0.74 g in 2.0 ml of water) was added to this refluxing solution. The reaction was kept at reflux for 1 hour. After cooling down to room temperature, concentrated HCl (3.0 ml) was added into the reaction mixture. The reaction solvents were removed. After the addition of water (80.0 ml), a white solid product was obtained and collected by filtration. After drying under vacuum, a white solid product was obtained in 1.14 g (98.3%) yield.

¹H NMR (400 MHz, DMSO-d₆) δ: 8.67 (t, 1H, J=1.6 Hz), 8.32 (d, 2H, J=7.6 Hz), 8.24 (d, 2H, J=8.4 Hz), 8.13 (d, 2H, J=8.4 Hz), 8.05 (d, 2H, J=8.4 Hz), 7.86 (t, 1H, J=7.6 Hz), 7.63 (d, 2H, J=8.4 Hz), 1.32 (s, 9H, 3×C H₃) ppm.

### Step 7: Bicyclo[2,2,1]hept-5en-2-ylmethyl 4-(5-(3-(5-(4-tert-butylphenyl)-1,3,4-oxadiazol-2-yl)phenyl)-1,3,4-oxadiazol-2-yl)benzoate (YZ-I-273)

To a solution of 4-(5-(3-(5-(4-tert-Butylphenyl)-1,3,4-oxadiazol-2-yl)phenyl)-1,3,4-oxadiazol-2-yl)benzoic acid (1.0 g, 2.14 mmol) and 5-(bromomethyl)bicycle[2,2,1]hept-2-ene (0.8 g, 4.28 mmol) in DMF (30.0 ml), K₂CO₃ (4.0 g, 28.94 mmol) was added at room temperature. The reaction was carried out at 80° C. for 30 hours. After cooling down to room temperature, the water (150.0 ml) was added into the reaction mixture. A pink solid precipitate was collected by filtration and washed with methanol and dried under vacuum. The crude product was purified by silica gel column chromatography, eluting with dichloromethane and ethyl acetate in a 15:1 ratio. After evaporating the solvent, the white solid was recrystallized from dichloromethane/methanol and finally dried under vacuum. A pure product was obtained as a white solid in 1.04 g (84.6%) yield.

¹H NMR (400 MHz, CDCl₃) δ: 8.89 (t, 1H, J=1.6 Hz), 8.36 (dd, 2H, J₁=8.0 Hz, J₂=1.6 Hz), 8.28 (d, 2H, J=8.0 Hz), 8.25 (d, 2H, J=8.0 Hz), 8.11 (d, 2H, J=8.4 Hz), 7.76 (t, 1H, J=8.0 Hz), 7.59 (d, 2H, J=8.4 Hz), 6.24-6.02 (m, 2H, C═C—H, end and exo), 4.49-3.94 (m, 2H, OCH₂, endo and exo), 3.02 (s, br), 2.89 (m, br), 2.85 (s, br), 2.59 (m, br), 1.94 (m), 1.53 (m), 1.38 (s, 9H, 3×CH3), 1.43-1.23 (m), 0.68 (m) ppm. ¹³C NMR (100 MHz, CDCl₃) δ: 165.50, 165.17, 164.30, 164.03, 163.30, 155.72, 137.80, 137.05, 136.16, 133.45, 133.37, 132.10, 130.30, 130.09, 129.99, 129.78, 127.23, 126.98, 126.91, 126.14, 125.22, 125.04, 124.72, 120.70, 69.55, 68.88, 49.42, 44.99, 43.96, 43.69, 42.20, 41.60, 38.03, 37.83, 35.13, 31.10, 29.60, 28.96 ppm. MS (m/z): [M+1]⁺ calcd for C₃₅H₃₂N₄O₄ 573.2, found 573.3. Anal. Calcd for C₃₅H₃₂N₄O₄: C, 73.41;H, 5.63; N, 9.78. Found: C, 73.20; H, 5.59; N, 9.67.

### Preparative Example 4

### Synthesis of YZ-I-275

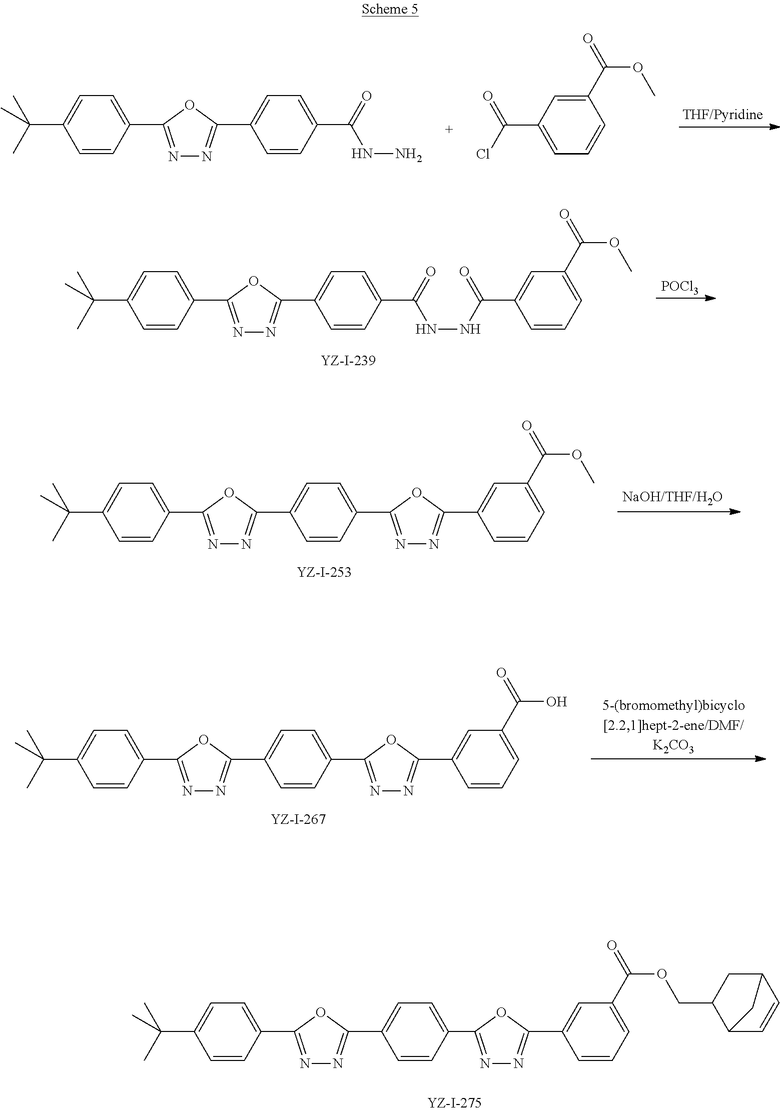

### Step 1: Methyl 3-(2-(4-(5-(4-tert-butylphenyl)-1,3,4-oxadiazol-2-yl)benzoyl)hydrazinecarbonyl)-benzoate (YZ-I-239)

To a solution of 4-(5-(4-tert-butylphenyl)-1,3,4-oxadiazol-2-yl)benzo-hydrazine (2.0 g, 5.95 mmol) in dry tetrahydrofuran (80.0 ml) was added methyl 3-(chlorocarbonyl)benzoate (1.2 g, 6.04 mmol) dropwise using a syringe. During the addition of methyl 3-(chlorocarbonyl)benzoate the solid appeared. The reaction mixture was stirred for 18 hours and then pyridine (10.0 ml) was added and stirred for another 1.5 hours. Then, water (300.0 ml) was added. The yellow solid was collected by filtration and dried overnight under vacuum and gave 2.67 g (90.2%) in yield.

¹H NMR (400 MHz, DMSO-d₆) δ: 10.83 (s, br, 2H, 2×NH), 8.35 (s, 1H), 8.30-7.95 (m, 8H), 7.70 (t, 1H, J=8.0 Hz), 7.65 (d, 2H, J=8.0 Hz), 3.90 (s, 3H, OCH₃), 1.32 (s, 9H, 3×CH₃) ppm.

### Step 2: Methyl3-(5-(4-(5-(4-tert-butylphenyl)-1,3,4-oxadiazol-2-yl)phenyl)-1,3,4-oxadiazol-2-yl)benzoate (YZ-I-253)

Methyl 3-(2-(4-(5-(4-tert-butylphenyl)-1,3,4-oxadiazol-2-yl)benzoyl)-hydrazinecarbonyl)benzoate (2.5 g, 5.01 mmol) was added in POCl₃ (25.0 ml). The reaction was heated to 90° C. and kept at this temperature for 2 hours. After cooling down to room temperature, the reaction mixture was slowly dropped into ice-water (300.0 ml). The yellow color solid that formed was collected by vacuum filtration. The crude material was purified by a silica gel column using dichloromethane/ethyl acetate, ratio of (9:1), as the eluent. After the removal of solvents, a pure product as a white solid was obtained in 1.22 g (50.6%) yield by recrystallization from dichloromethane/methanol.

¹H NMR (400 MHz, CDCl₃) δ: 8.80 (t 1H, J=1.6 Hz), 8.39 (dt, 1H, J₁=8.0 Hz, J₂=1.6 Hz), 8.34 (s, 2H), 8.33 (s, 2H), 8.25 (dt, 1H, J₁=8.0 Hz, J₂=1.6 Hz), 8.09 (d, 2H, J=8.4 Hz), 7.67 (t, 1H, J=8.0 Hz), 7.58 (d, 2H, J=8.4 Hz), 4.00 (s, 3H, OCH₃), 1.39 (s, 9H, 3×CH₃) ppm. ¹³C NMR (100 MHz, CDCl₃) δ: 165.94, 165.16, 164.22, 164.02, 163.42, 155.73, 132.82, 131.29, 131.14, 129.44, 1 27.98, 127.58, 127.48, 126.88, 126.19, 126.13, 124.02, 120.70, 52.55, 35.12, 31.08 ppm. MS-FAB (m/z): [M]⁺ calcd for C₂₈H₂₄N₄O₄ 480.2, found 480.8.

### Step 3: 3-(5-(4-(5-(4-tert-Butylphenyl)-1,3,4-oxadiazol-2-yl)phenyl)-1,3,4-oxadiazol-2-yl)benzoic acid (YZ-I-267)

Methyl 3-(5-(4-(5-(4-tert-butylphenyl)-1,3,4-oxadiazol-2-yl)phenyl)-1,3,4-oxadiazol-2-yl)benzoate (1.1 g, 2.29 mmol) was taken into THF (150.0 ml) and ethanol (35.0 ml). The reaction mixture was heated to reflux. NaOH (0.76 g in 3.0 ml of water) was added to this refluxing solution. The reaction was kept at reflux for 1 hour. After cooling down to room temperature, concentrated HCl (3.0 ml) was added into the reaction mixture. The reaction solvents were removed. Then, water (80.0 ml) was added and a white solid product was obtained and collected by filtration. After drying under vacuum, a white solid product was obtained in 1.02 g (95.3%) yield.

¹H NMR (400 MHz, DMSO-d₆) δ: 8.59 (s, 1H), 8.31 (m, 5H), 8.15 (d, 1H, J=7.6 Hz), 8.03 (d, 2H, J=8.8 Hz), 7.75 (t, 1H, J=7.6 Hz), 7.62 (d, 2H, J=8.8 Hz), 1.32 (s, 9H, 3×CH₃) ppm.

### Step 4: Bicyclo[2,21]help-5-en-2-ylmethyl 3-(5-(4-(5-(4-tert-butylphenyl)-1,3,4-oxadiazol-2yl)-phenyl)-1,3,4-oxadiazol-2-yl)benzoate (YZ-I-275)

To a solution of 3-(5-(4-(5-(4-tert-butylphenyl)-1,3,4-oxadiazol-2-yl)phenyl)-1,3,4-oxadiazol-2-yl)benzoic acid (1.0 g, 2.14 mmol) and 5-(bromomethyl)bicycle[2,2,1]hept-2-ene (0.8 g, 4.28 mmol) in DMF (35.0 ml) was added K₂CO₃ (8.0 g, 57.88 mmol) at room temperature. The reaction was carried out at 100° C. for 30 hours. After cooling down to room temperature the reaction mixture was poured into water (100.0 ml). A brown solid precipitate was obtained by filtration and washed with methanol and dried under vacuum. The crud e material was purified by silica gel column chromatography, eluting with dichloromethane and ethyl acetate in a 15:1 ratio. After evaporating the solvent, the white solid was recrystallized from dichloromethane/methanol and finally dried under vacuum. A pure product was obtained as a white solid in 0.91 g (74.0%) yield.

¹H NMR (400 MHz, CDCl₃) δ: 8.81 (m, 1H), 8.39 (m, 1H), 8.34 (s, 4H), 8.25 (m, 1H), 8.10 (d, 2H, J=8.4 Hz), 7.67 (m, 1H), 7.58 (d, 2H, J=8.4 Hz), 6.23 (q, 0.72 H_{endo}, J=3.2 Hz), 6.15 (m, 0.56 Hₑₓₒ), 6.04 (q, 0.72 H_{endo}, J=3.2 Hz), 4.48 (dd, 0.28 Hₑₓₒ, 2/14×OCH₂, J₁=10.8 Hz, J₂=6.4 Hz), 4.31 (dd, 0.28 Hₑₓₒ, 2/14×OCH₂, J₁=10.6 Hz, J₂=9.2 Hz), 4.18 (dd, 0.72 H _{endo}, 5/14×OCH₂, J₁=10.8 Hz, J₂=6.4 Hz), 4.00 (dd, 0.72 H_{endo}, 5/14×OCH₂, J₁=10.6 Hz, J₂=9.2 Hz), 3.02 (s, br), 2.89 (m, br), 2.61 (m, br), 1.96 (m), 1.53 (m), 1.38 (s, 9H, 3×CH3), 1.43-1.23 (m), 0.70 (m) ppm. ¹³C NMR (100 MHz, CDCl₃) δ: 165.43, 165.18, 164.29, 164.04, 163.44, 155.73, 137.82, 137.07, 132.81, 132.13, 131.72, 131.04, 129.40, 128.02, 127.60, 127.50, 126.90, 126.23, 126.15, 124.03, 120.71, 69.60, 68.94, 49.44, 45.02, 43.99, 43.71, 42.22, 41.62, 38.06, 37.86, 35.13, 31.09, 29.62, 28.99 ppm. MS (m/z): [M+1]⁺ calcd for C₃₅H₃₂N₄O₄ 573.3, found 573.3. Anal. Calcd for C₃₅H₃₂N₄O₄: C, 73.41; H, 5.63; N, 9.78. Found: C, 73.18; H, 5.63; N, 9.63.

### Preparative Example 5

### Synthesis of YZ-I-277

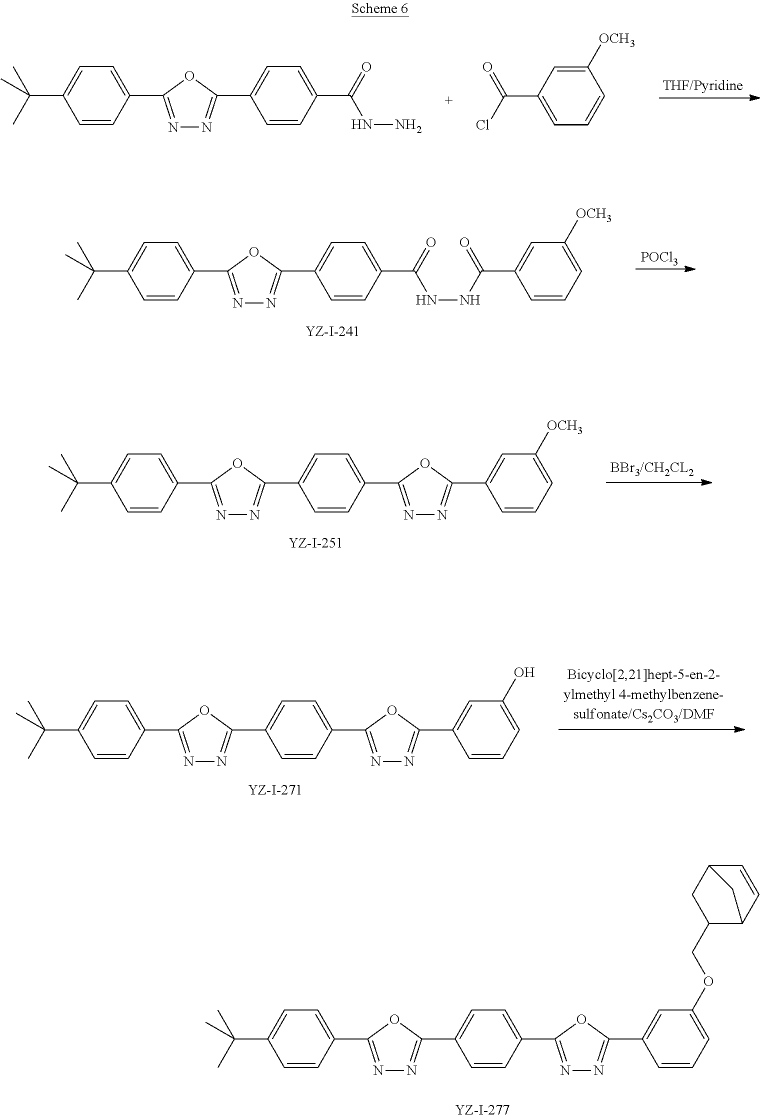

### Step 1: N′-(4-(5-(4-tert-Butylphenyl)-1,3,4-oxadiazol-2-yl)benzoyl)-3-methoxybenzohydrazide (YZ-I-241)

To a solution of 4-(5-(4-tert-butylphenyl)-1,3,4-oxadiazol-2-yl)benzohydrazine (2.0 g, 5.95 mmol) in dry tetrahydrofuran (80.0 ml) and DMF (7.0 ml) was slowly added 3-methoxybenzoyl chloride (1.2 g, 7.03 mmol) at room temperature. During the addition of 3-methoxybenzoyl chloride, white solids appeared. The reaction mixture was stirred for 18 hours and then pyridine (10.0 ml) was added and stirred for another 2 hours. Next, water (300.0 ml) was added. The yellow solid obtained was collected by filtration and dried overnight under vacuum provided 2.70 g (96.4%) in yield.

¹H NMR (400 MHz, CDCl₃) δ: 10.64 (s, br, 2H, 2×NH), 8.28 (d, 2H, J=8.4 Hz), 8.20-7.90 (m, 5H), 7.65 (m, 2H), 7.53-7.43 (m, 2H), 7.17 (m, 1H), 3.83 (s, 3H, OCH₃), 1.33 (s, 9H, 3×CH₃) ppm.

### Step 2: 2-(4-tert-Butylphenyl)-5-(4-(5-(3-methoxyphenyl)-1,3,4-oxadizol-2-yl)phenyl)-1,3,4-oxadiazole (YZ-I-251)

N′-(4-(5-(4-tert-Butylphenyl)-1,3,4-oxadiazol-2-yl)benzoyl)-3-methoxybenzohydrazine (2.5 g, 5.31 mmol) was added in POCl₃ (25.0 ml). The reaction was heated to 90° C. and kept at this temperature for 4 hours. After cooling down to room temperature, the reaction mixture was slowly dropped into ice-water (300.0 ml). The yellow color solid for med was collected by vacuum filtration. The crude material was dried and purified by silica gel column using dichloromethane/ethyl acetate, ratio (9:1), as the eluent. After removal of the solvents, a pure product as white solid was obtained in 1.43 g (59.6%) yield by recrystallization from THF/methanol.

¹H NMR (400 MHz, CDCl₃) δ: 8.32 (s, 4H), 8.08 (d, 2H, J=8.8 Hz), 7.72 (dt, 1H, J₁=8.0 Hz, J₂=1.2 Hz), 7.69 (dd, 1H, J₁=2.4 Hz, J₂=1.2 Hz), 7.57 (d, 2H, J=8.8 Hz), 7.46 (t, 1H, J=8.0 Hz), 7.12 (ddq, 1H, J₁=8.0 Hz, J₂ =2.4 Hz, J₃=1.2 Hz), 3.92 (s, 3H, OCH₃), 1.39 (s, 9H, 3×CH₃) ppm. ¹³C NMR (100 MHz, CDCl₃) δ: 165.12, 164.94, 163.71, 163.45, 159.97, 155.70, 130.28, 127.47, 127.42, 126.87, 126.70, 126.40, 126.13, 124.65, 120.71, 119.38, 118.39, 111.67, 55.54, 35.12, 31.08 ppm. MS-FAB (m/z): [M]⁺ calcd for C₂₈H₂₄N₄O₄ 452.2, found 452.2.

### Step 3: 3-(5-(4-(5-(4-tert-Butylphenyl)-1,3,4-oxadiazol-2-yl)phenyl)-1,3,4-oxadiazol-2-yl)phenol (YZ -I-271)

To a solution of 2-(4-tert-butylphenyl)-5-(4-(5-(3-methoxyphenyl)-1,3,4-oxadizol-2-yl)phenyl)-1,3,4-oxadiazole (1.2 g, 2.65 mmol) in dichloromethane (50.0 ml), was dropwise added BBr₃ (16.0 ml, 1 M in dichloromethane) at −78° C. (dry-ice/acetone) under nitrogen. After the addition of BBr₃ solution, the reaction was taken to room temperature and kept at room temperature for 5 hours. The reaction mixture was poured into ice-water (100.0 ml). Dichloromethane was evaporated under reduced pressure. The white solid was collected by filtration. After drying under vacuum, a white solid product was obtained in 1.1 g (94.8%) yield.

¹H NMR (400 MHz, DMSO-d₆) δ: 10.03 (s, 1H), 8.32 (s, 4H), 8.06 (d, 2H, J=8.4 Hz), 7.64 (d, 2H, J=8.4 Hz), 7.57 (d, 1H, J=7.6 Hz), 7.52 (m, 1H), 7.43 (t, 1H, J=7.6 Hz), 7.03 (d, 1H, J=7.6 Hz), 1.32 (s, 9H, 3×CH₃) ppm.

### Step 4: 2-(3-(Bicyclo [2,2,1]hept-5-en-2-ylmethoxy)phenyl)-5-(4-(5-(4-tert-butylphenyl)-1,3,4-oxadiazol-2-yl)phenyl)-1,3,4-oxadiazole (YZ-I-277)

To a solution of 3-(5-(4-(5-(4-tert-butylphenyl)-1,3,4-oxadiazol-2-yl)phenyl)-1,3,4-oxadiazol-2-yl)phenol (1.0 g, 2.28 mmol) and bicyclo[2,2,1]hept-5-en-2-ylmethyl 4-methylbenzenesulfonate (1.6 g, 5.75 mmol) in DMF (50.0 ml), was added Cs₂CO₃ (5.0 g, 15.35 mmol) at room temperature. The reaction was carried out at 100° C. for 5 hours. After cooling down to room temperature, the reaction mixture was poured into water (150.0 ml). A brown solid precipitate was obtained by filtration and dried under vacuum. The crude product was purified by a silica gel column using dichloromethane/ethyl acetate (9.3:0.7) as the eluent. After removal of the solvents, a pure white solid product was obtained in 1.1 g (88.7%) yield by recrystallization from dichloromethane/methanol.

¹H NMR (400 MHz, CDCl₃) δ: 8.32 (s, 2H), 8.31 (s, 2H), 8.10 (d, 2H, J=8.4 Hz), 7.74-7.659 (m, 2H), 7.58 (d, 2H, J=8.4 Hz), 7.45 (m, 1H), 7.11 (m, 1H), 6.22-6.12 (m, 2H, C═C—H, endo, exo), 4.16-3.63 (m, 2H, OCH₂, endo, exo), 3.08 (s, br), 2.89 (m, br), 2.62 (m, br), 1.96 (m), 1.51 (m), 1.39 (s, 9H, 3×CH₃), 1.40-1.23 (m), 0.67 (m) ppm. ¹³C NMR (100 MHz, CDCl₃) δ: 165.14, 165.02, 163.72, 163.47, 159.57, 155.71, 137.70, 136.92, 136.36, 132.27, 130.27, 130.21, 127.49, 127.44, 126.89, 126.70, 126.45, 126.13, 124.61, 120.7 3, 119.25, 119.15, 118.87, 112.33, 72.58, 71.78, 49.43, 45.06, 43.88, 43.70, 42.23, 41.60, 38.53, 38.32, 35.13, 31.09, 29.63, 29.00 ppm. MS (m/z): [M+1]⁺ calcd for C₃₄H₃₂N₄O₃ 545.3, found 545.3. Anal. Calcd for C₃₄H₃₂N₄O₃: C, 74.981; H, 5.92; N, 10.29. Found: C, 75.03; H, 5.78; N, 10.25.

### Preparative Example 6

### Synthesis of YZ-I-279

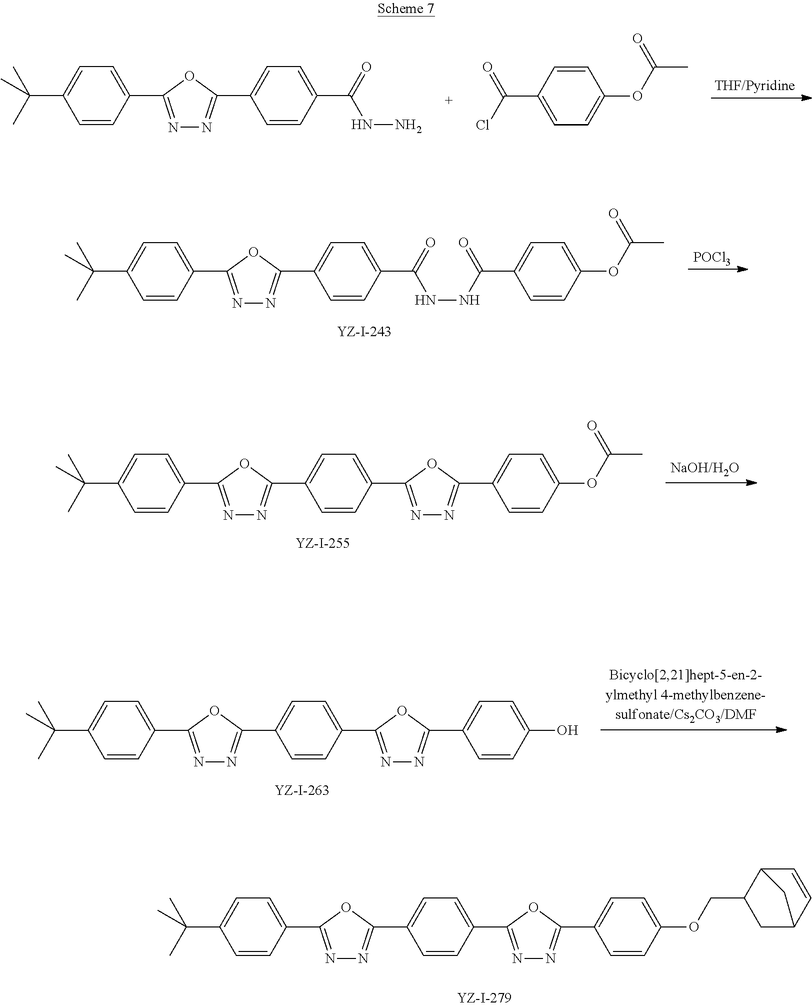

### Step 1: 4-(2-(4-(5-(4-tert-butylphenyl)-1,3,4-oxadiazol-2-yl)benzoyl)hydrazinecarbonyl)phenyl acetate (YZ-I-243)

To a solution of 4-(5-(4-tert-butylphenyl)-1,3,4-oxadiazol-2-yl)benzohydrazine (2.0 g, 5.95 mmol) in dry tetrahydrofuran (80.0 ml) and DMF (7.0 ml), was slowly added 4-(chlorocarbonyl)phenyl acetate (1.3 g, 6.55 mmol) at room temperature. During addition of 4-(chlorocarbonyl)phenyl acetate, white solids appeared. The reaction mixture was stirred for 19 hours and then pyridine (10.0 ml) was added and stirred for another 1.5 hours. Next, water (300.0 ml) was added. The yellow solid was collected by filtration and dried overnight under vacuum and provided 2.70 g (90.0%) in yield.

¹H NMR (400 MHz, DMSO-d₆) δ: 10.79 (s, 1H, NH), 10.64 (s, 1H, NH), 8.28 (d, 2H, J=8.4 Hz), 8.15 (d, 2H, J=8.4 Hz), 8.08 (d, 2H, J=8.0 Hz), 7.97 (d, 2H, J=8.8 Hz), 7.66 (d, 2H, J=8.4 Hz), 7.30 (d, 2H, J=8.8 Hz), 2.31 (s, 3H, CH₃), 1.33 (s, 9H, 3×CH₃) ppm.

### Step 2: 4-(5-(4-(5-(4-tert-Butylphenyl)-1,3,4-oxadiazol-2-yl)phenyl)-1,3,4-oxadiazol-2-yl)phenyl acetate (YZ-I-255)

4-(2-(4-(5-(4-tert-butylphenyl)-1,3,4-oxadiazol-2-yl)benzoyl)hydrazine-carbonyl)phenyl acetate (2.5 g, 5.01 mmol) was added in POCl₃ (25.0 ml). The reaction was heated to 90° C. and kept at this temperature for 2 hours. After cooling down to room temperature, the reaction mixture was slowly dropped into ice-water (300.0 ml). The yellow color solid that formed was collected by vacuum filtration. The crude material was dried and purified by silica gel column using dichloromethane/ethyl acetate, ratio (9:1), as the eluent. After removal of the solvents, a pure product as a white solid was obtained in 0.86 g (35.8%) yield by recrystallization from THF/methanol.

¹H NMR (400 MHz, CDCl₃) δ: 8.32 (s, 4H), 8.20 (d, 2H, J=8.0 Hz), 8.09 (d, 2H, J=8.4 Hz), 7.57 (d, 2H, J=8.0 Hz), 7.31 (d, 2H, J=8.4 Hz), 2.36(s, 3H, Ch₃), 1.39 (s, 9H, 3×CH₃) ppm. ¹³C NMR (100 MHz, CDCl₃) δ: 168.87, 165.14, 164.35, 163.78, 163.45, 155.71, 153.44, 128.43, 127.48, 127.46, 126.88, 126.75, 126.35, 126.13, 122.55, 121.17, 120.71, 35.12, 31.08, 21.15 ppm. MS -FAB (m/z): [M]⁺ calcd for C₂₈H₂₄N₄O₄ 480.2, found 480.7.

### Step 3: 4-(5-(4-(5-(4-tert-Butylphenyl)-1,3,4-oxadiazol-2-yl)phenyl)-1,3,4-oxadiazol-2-yl)phenol (YZ-I-263)

4-(5-(4-(5-(4-tert-Butylphenyl)-1,3,4-oxadiazol-2-yl)phenyl)-1,3,4-oxadiazol-2-yl)phenyl acetate (0.8 g, 1.66 mmol) was taken into THF (150.0 ml). The reaction mixture was heated to reflux. When the starting material was dissolved in THF, NaOH (0.3 g in 1.5 ml of water) was added to this refluxing solution. During the addition of NaOH, the color of the reaction solution changed to yellow. The reaction was kept at reflux for 1 hour, and heating was stopped. After cooling down to room temperature, concentrated HCl (3.0 ml) was added into the reaction mixture. Then, the reaction solvents were removed. After the addition of water (80.0 ml), a white solid product was obtained and collected by filtration. After drying under vacuum, a white solid product was obtained in 0.72 g (98.6%) yield.

¹H NMR (400 MHz, DMSO-d₆) δ: 10.37 (s, br, 1H, OH), 8.25 (s, 4H), 8.02 (d, 2H, J=8.4 Hz), 7.94 (d, 2H, J=8.8 Hz), 7.61 (d, 2H, J=8.8 Hz), 6.96 (d, 2H, J=8.4 Hz), 1.31 (s, 9H, 3×CH₃) ppm.

### Step 4: 2-(4-(Bicycle[2,2,1]hept-5-en-2ylmethoxy)phenyl)-5-(4-(5-(4-tert-butylphenyl)-1,3,4-oxadiazol-2-yl)phenyl)-1,3,4-oxadiazole (YZ-I-279)

To a solution of 4-(5-(4-(5-(4-tert-butylphenyl)-1,3,4-oxadiazol-2-yl)phenyl)-1,3,4-oxadiazol-2-yl)phenol (0.70 g, 1.60 mmol) and bicyclo[2,2,1]hept-5-en-2-ylmethyl 4-methylbenzenesulfonate (1.0 g, 3.59 mmol) in DMF (50.0 ml), was added Cs₂CO₃ (3.8 g, 11.66 mmol) at room temperature. The reaction was carried out at 100° C. for 3 hours. After cooling down to room temperature, the reaction was poured into water (150.0 ml). A white solid precipitate was obtained by filtration and washed with methanol and dried under vacuum. The product was obtained in 0.77 g (88.5%) yield.

### Preparative Example 7

### Synthesis of YZ-I-281

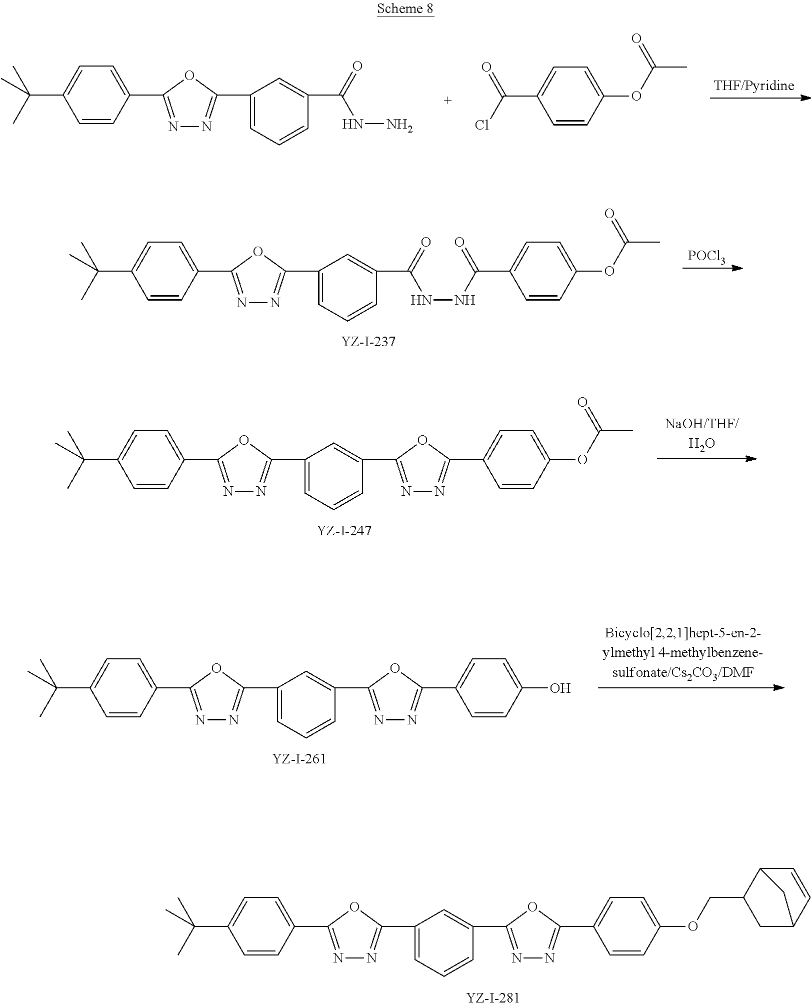

### Step 1: 4-(2-(3-(5-(4-tert-butylphenyl)-1,3,4-oxadiazol-2-yl)benzoyOhydrazinecarbonyl)phenyl acetate (YZ-I-237)

To a solution of 3-(5-(4-tert-butylphenyl)-1,3,4-oxadiazol-2-yl)benzo-hydrazine (2.0 g, 5.95 mmol) in dry tetrahydrofuran (100.0 ml) and DMF (5.0 ml), was slowly added methyl 4-(chlorocarbonyl)phenyl acetate (1.3 g, 6.54 mmol) at room temperature under nitrogen. During the addition of methyl 3-(chlorocarbonyl)phenyl acetate, white solids appeared. The reaction mixture was stirred at room temperature for 18 hours and then pyridine (10.0 ml) was added and stirred for another hour. Then, water (300.0 ml) was added into the reaction mixture. The white solid was collected by filtration and dried overnight under vacuum and provided 2.7 g (90.9%) yield.

¹H NMR (400 MHz, DMSO-d₆) δ: 10.88 (s, br, 2H, 2×NH), 8.52 (t, 1H, J=1.6 Hz), 8.22 (dt, 2H, J₁=7.6 Hz, J₂=1.6 Hz), 8.05 (m, 4H), 7.71 (t, 1H, J=7.6 Hz) 7.65 (m, 4H), 2.49 (s, 3H, CH₃), 1.33 (s, 9H, 3×CH₃) ppm.

### Step 2: 4-(5-(3-(5-(4-tert-Butylphenyl)-1,3,4-oxadiazol-2-yl)phenyl)-1,3,4-oxadiazol-2-yl)phenyl acetate (YZ-I-247)

4-(2-(3-(5-(4-tert-butylphenyl)-1,3,4-oxadiazol-2-yl)-benzoyl)-hydrazinecarbonyl)phenyl acetate (2.1 g, 4.21 mmol) was added in POCl₃ (30.0 ml). The reaction was heated to 90° C. and kept at this temperature for 3 hours. After cooling down to room temperature, the reaction mixture was slowly dropped into ice-water (300.0 ml). The white solid formed was collected by vacuum filtration. The crude product was dried and purified by silica gel column using dichloromethane/ethyl acetate, ratio (8.5:1.5), as t he eluent. After the removal of the solvents, a pure white solid product was obtained in 1.23 g (60.9%) yield.

¹H NMR (400 MHz, CDCl₃) δ: 8.86 (t, 1H, J=1.6 Hz), 8.34 (m, 2H), 8.22 (d, 2H, J=8.8 Hz), 8.12 (d, 2H, J=8.8 Hz), 7.74 (t, 1H, J=7.6 Hz), 7.58 (d, 2H, J=8.8 Hz), 7.32 (d, 2H, J=8.8 Hz), 2.36 (s, 3H, CH₃), 1.39 (s, 9H, 3×CH₃) ppm. NMR (100 MHz, CDCl₃) δ: 169.16, 165.41, 164.63, 163.96, 163.63, 162.04, 155.95, 153.71, 130.30, 130.07, 129.95, 128.75, 127.18, 126.41, 125.43, 125.22, 125.16, 122.82, 121.45, 120.99, 35.40, 31.36, 21.43 ppm. MS -EI (m/z): [M]⁻ calcd for C₂₈H₂₄N₄O₄ 480.2, found 480.2.

### Step 3: 4-(5-(3-(5-(4-tert-butylphenyl)-1,3,4-oxadiazol-2-yl)phenyl)-1,3,4-oxadiazol-2-yl)phenol (YZ-I-261)

4-(5-(3-(5-(4-tert-Butylphenyl)-1,3,4-oxadiazol-2-yl)phenyl)-1,3,4-oxadiazol-2-yl)-phenyl acetate (1.2 g, 2.50 mmol) and NaOH (0.2 g, in 1.0 ml of water) were taken into THF (35.0 ml). The reaction was heated to reflux and kept at reflux for 1 hour. During reflux, yellow solids appeared. After cooling down to room temperature, concentrated HCl (3.0 ml) was added into the reaction mixture. After the removal of the reaction solvents, water (80.0 ml) was added. The white solid product was collected by filtration. After drying under vacuum, a white solid product was obtained in 1.10 g (100%) yield.

¹H NMR (400 MHz, DMSO-d₆) δ: 10.38 (s, 1H), 8.67 (s, 1H), 8.31 (m, 2H), 8.07 (d, 2H, J=8.4 Hz), 7.99 (d, 2H, J=8.4 Hz), 7.85 (t, 1H, J=7.6 Hz), 7.63 (d, 2H, J=8.4 Hz), 6.98 (d, 2H, J=8.4 Hz), 1.33 (s, 9H, 3×CH₃) ppm.

### Step 4: 2-(4-(Bicyclo [2,2,1]hept-5-en-2-ylmethoxy)phenyl)-5-(3-(5-(4-tert-butylphenyl)-1,3,4-oxadiazol-2-yl)phenyl)-1,3,4-oxadiazole (YZ-I-281)

To a solution of 4-(5-(3-(5-(4-tert-butylphenyl)-1,3,4-oxadiazol-2-yl)phenyl)-1,3,4-oxadiazol-2-yl)phenol (1.0 g, 2.28 mmol) and bicyclo[2,2,1]hept-5-en-2-ylmethyl 4-methylbenzenesulfonate (1.6 g, 5.75 mmol) in DMF (50.0 ml), was added Cs₂CO₃ (4.24 g, 13.01 mmol) at room temperature under nitrogen. The reaction was carried out at 100° C. for 2 hours. After cooling down to room temperature, water (150.0 ml) was added into the reaction mixture. A brown solid precipitate was collected by filtration and washed with methanol and then dried under vacuum. The crude product was purified by silica gel column using dichloromethane/ethyl acetate, ratio (9.3:0.7), as the eluent. After removal of the solvents, a pure white solid product was obtained in 1.12 g (90.3%) yield by recrystallization from dichloromethane/methanol.

¹H NMR (400 MHz, CDCl₃) δ: 8.85 (t, 1H, J=1.6 Hz), 8.32 (dd, 2H, J₁=7.6 Hz, J₂=1.6 Hz), 8.10 (m, 4H), 7.72 (t, 1H, J=7.6 Hz), 7.58 (d, 2H, J=8.4 Hz), 7.05 (m, 2H), 6.22-5.98 (m, 2H, C═C—H, endo and exo), 4.14-3.62 (m, 2H, OCH₂, endo and exo), 3.07 (s, br), 2.89 (m, br), 2.60 (m, br), 1.95 (m), 1.50 (m), 1.39 (s, 9 H, 3×CH₃), 1.40-1.23 (m), 0.67 (m) ppm. ¹³C NMR (100 MHz, CDCl₃) δ: 165.11, 165.02, 163.43, 163.12, 162.14, 155.64, 137.75, 136.92, 136.33, 132.20, 129.94, 129.56, 129.53, 128.83, 128.79, 126.90, 126.12, 125.16, 125.06, 124.81, 120.75, 115.83, 115.05, 72.47, 71.71, 49.43, 45.04, 43.85, 43.66, 42.21, 41.58, 38.46, 38.24, 35.11, 31.09, 29.60, 28.99 ppm. MS (m/z): [M+1]⁺ calcd for C₃₄H₃₂N₄O₃ 545.3, found 545.2. Anal. Calcd for C₃₄H₃₂N₄O₃: C, 74.98; H, 5.92; N, 10.29. Found: C, 74.99; H, 5.76; N, 10.18.

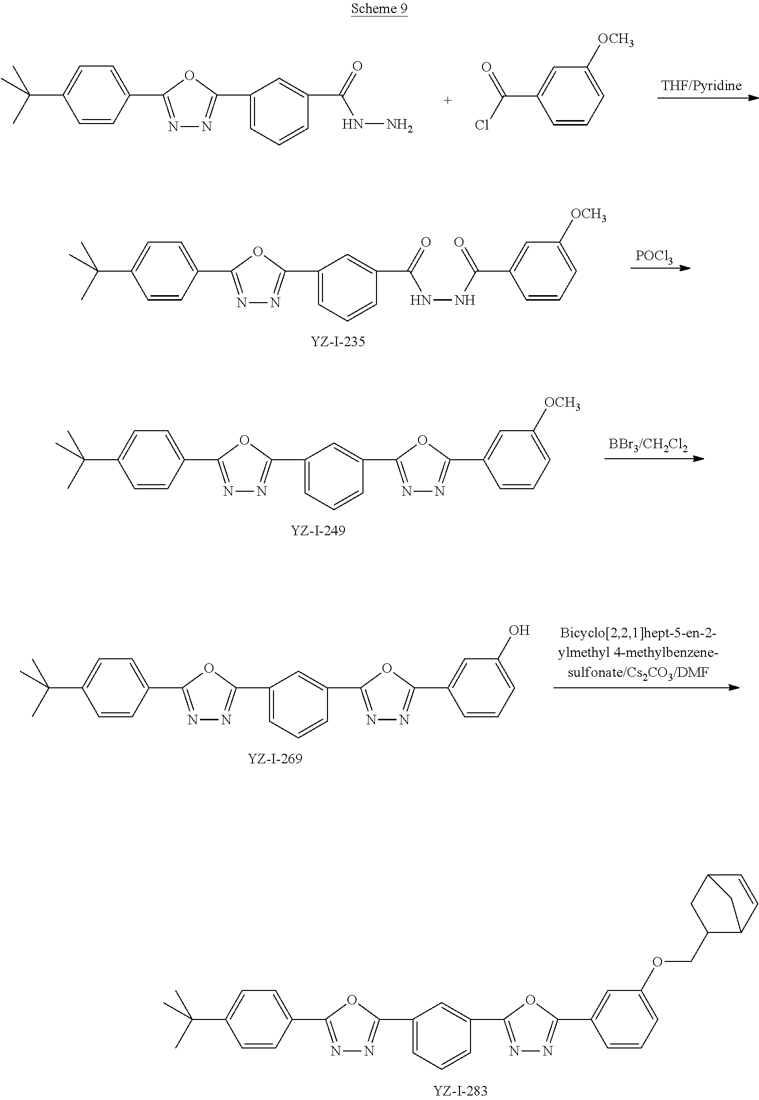

### Preparative Example 8

### Synthesis of YZ-I-283

Step 1: 3-(5-(4-tert-butylphenyl)-1,3,4-oxadiazol-2-yl)-N′-(3-methyoxybenzoyl)benzohydrazine (YZ-I-235)

To a solution of 3-(5-(4-tert-butylphenyl)-1,3,4-oxadiazol-2-yl)benzohydrazine (1.5 g, 4.46 mmol) in dry tetrahydrofuran (50.0 ml) and DMF (5.0 ml), was slowly added 3-methoxybenzoyl chloride (0.8 g, 4.69 mmol) at room temperature under nitrogen. During addition of 3-methoxybenzoyl chloride, white solids appeared. The reaction mixture was stirred at room temperature for 21 hours and then pyridine (10.0 ml) was added and stirred for another hour. Then, water (300.0 ml) was added into the reaction mixture. The white solid was collected by filtration and dried overnight under vacuum and provided 1.9 g (90.4%) yield.

¹H NMR (400 MHz, DMSO-d₆) δ: 10.83 (s, br, 1H, NH), 10.64 (s, br, NH), 8.66(s, 1H), 8.34 (d, 1H, J=7.6 Hz), 8.17 (d, 1H, J=7.6 Hz), 8.07 (d, 2H, J=8.0 Hz), 7.80 (t, 1H, J=7.6 Hz), 7.65 (d, 2H, J=8.0 Hz), 7.54-7.43 (m, 3H), 7.17 (d, 1H, J=8.0 Hz), 3.83 (s, 3H, OCH₃), 1.33 (s, 9H, 3×CH₃) ppm.

### Step 2: 2-(4-tert-Butylphenyl)-5-(3-(5-(3-methoxyphenyl)-1,3,4-oxadiazol-2-yl)phenyl)-1,3,4-oxadiazole (YZ-I-249)

3-(5-(4-tert-Butylphenyl)-1,3,4-oxadiazol-2-yl)-N′-(3-methyoxy-benzoyl)benzohydrazine (1.75 g, 3.72 mmol) was added in POCl₃ (15.0 ml). The reaction was heated to 90° C. and kept at this temperature for 4 hours. After cooling down to room temperature, the reaction mixture was slowly dropped into ice-water (300.0 ml). The white solid formed was collected by vacuum filtration. The crude product was dried and purified by a silica gel column using dichlorom ethane/ethyl acetate, ratio (9:1), as the eluent. After the removal of the solvents, a pure white solid product was obtained in 1.18 g (70.2%) yield.

¹H NMR (400 MHz, CDCl₃) δ: 8.86 (t, 1H, J=1.6 Hz), 8.34 (dt, 2H, J₁=7.6 Hz, J₂=1.6 Hz), 8.11 (d, 2H, J=8.4 Hz), 7.73 (m, 3H), 7.57 (d, 2H, J=8.4 Hz), 7.47 (t, 1H, J=7.6 Hz), 7.32 (dd, 1H, J₁=7.6 Hz, J₂=1.6 Hz), 3.93 (s, 3H, OCH₃), 1.39 (s, 9H, 3×CH₃) ppm. ¹³C NMR (100 MHz, CDCl₃) δ: 165.11, 164.94, 163.62, 163.34, 159.95, 155.64 , 130.26, 129.97, 129.74, 126.89, 126.10, 125.10, 124.92, 124.90, 124.65, 120.70, 119.42, 118.42, 111.60, 55.56, 35.10, 31.08 ppm. MS-EI (m/z): [M]⁺calcd for C₂₈H₂₄N₄O₄ 452.2, found 452.2.

### Step 3: 3-(5-(3-(5-(4-tert-Butylphenyl)-1,3,4-oxadiazol-2-yl)phenyl)-1,3,4-oxadiazol-2-yl)phenol (YZ-I-269)

To a solution of 2-(4-tert-butylphenyl)-5-(3-(5-(3-methoxyphenyl)-1,3,4-oxadiazol-2-yl)phenyl)-1,3,4-oxadiazole (1.0 g, 2.21 mmol) in dichloromethane (30.0 ml), was dropwise added BBr₃ (16.0 ml, 1 M in dichloromethane) at −78° C. (dry-ice/acetone) under nitrogen. After the addition of the BBr₃ solution, the reaction was taken to room temperature and kept at room temperature for 7 hours.

The reaction mixture was poured into ice-water (150.0 ml). Dichloromethane w as evaporated under reduced pressure. The white solid was collected by filtration. After drying under vacuum, a white solid product was obtained in 0.98 g (100%) yield.

δ: 10.02 (s, 1H), 8.68 (s, 1H), 8.31 (m, 2H), 8.07 (d, 2H, J=8.4 Hz), 7.86 (t, 1H, J=8.0 Hz), 7.63 (d, 2H, J=8.4 Hz), 7.58 (d, 1H, J=7.6 Hz), 7.53 (s, 1H), 7.42 (t, 1H, J=7.6 Hz), 7.03 (dd, 1H, J₁=7.6 Hz, J₂=1.6 Hz), 1.32 (s, 9H, 3×CH₃) ppm.

### Step 4: 2-(3-(Bicyclo[2,2,1]hept-5en-2-ylmethoxy)phenyl)-5-(3-(5-(4-tert-butylphenyl)-1,3,4-oxadiazol-2-yl)phenyl)-1,3,4-oxadiazole (YZ-I-283)

To a solution of 3-(5-(3-(5-(4-tert-butylphenyl)-1,3,4-oxadiazol-2-yl)phenyl)-1,3,4-oxadiazol-2-yl)phenol (0.92 g, 2.10 mmol) and bicyclo[2,2,1]hept-5-en-2-ylmethyl 4-methylbenzenesulfonate (1.6 g, 5.75 mmol) in DMF (45.0 ml), was added Cs₂CO₃ (4.5 g, 13.81 mmol) at room temperature under nitrogen. The reaction was carried out at 100° C. for 2 hours. After cooling down to room temperature, water (100.0 ml) was added into the reaction mixture. A brown solid precipitate was collected by filtration and washed with methanol and then dried under vacuum. The crude product was purified by a silica gel column using dichloromethane/ethyl acetate, ratio (9.3:0.7), as the eluent. After removal of the solvents, a pure white solid product was obtained in 0.97 g (85.1%) yield by recrystallization from dichloromethane/methanol.

¹H NMR (400 MHz, CDCl₃) δ: 8.86 (m, 1H), 8.34 (dd, 2H, J₁=8.0 Hz, J₂=1.6 Hz), 8.11 (d, 2H, J=8.4 Hz), 7.73 (m, 2H), 7.67 (m, 1H), 7.58 (d, 2H, J=8.4 Hz), 7.45 (m, 1H), 7.12 (m, 1H), 6.22-5.99 (m, 2H, C═C—H, endo and exo), 4.17-3.64 (m, 2H, OCH₂, endo and exo), 3.09 (s, br), 2.91 (m, br), 2.61 (m, br), 1.95 (m), 1.52 (m), 1.39 (s, 9H, 3×CH₃), 1.40-1.23 (m), 0.68 (m) ppm. ¹³C NMR (100 MHz, CDCl₃) δ: 165.14, 163.65, 163.38, 159.57, 155.67, 137.68, 136.90, 136.38, 132.29, 130.26, 129.99, 129.77, 129.71, 126.92, 126.13, 125.13, 124.98, 124.94, 124.61, 120.73, 119.31, 119.22, 118.90, 112.29, 72.57, 71.7 8, 49.42, 45.06, 43.87, 43.69, 42.23, 41.60, 38.54, 38.32, 35.12, 31.10, 29.62, 28.99 ppm. MS (m/z): [M+1]⁺ calcd for C₃₄H₃₂N₄O₃ 545.3, found 545.2. Anal. Calcd for C₃₄H₃₂N₄O₃: C, 74.98;H, 5.92; N, 10.29. Found: C, 74.77; H, 6.02; N, 10.27.

### Preparative Example 9

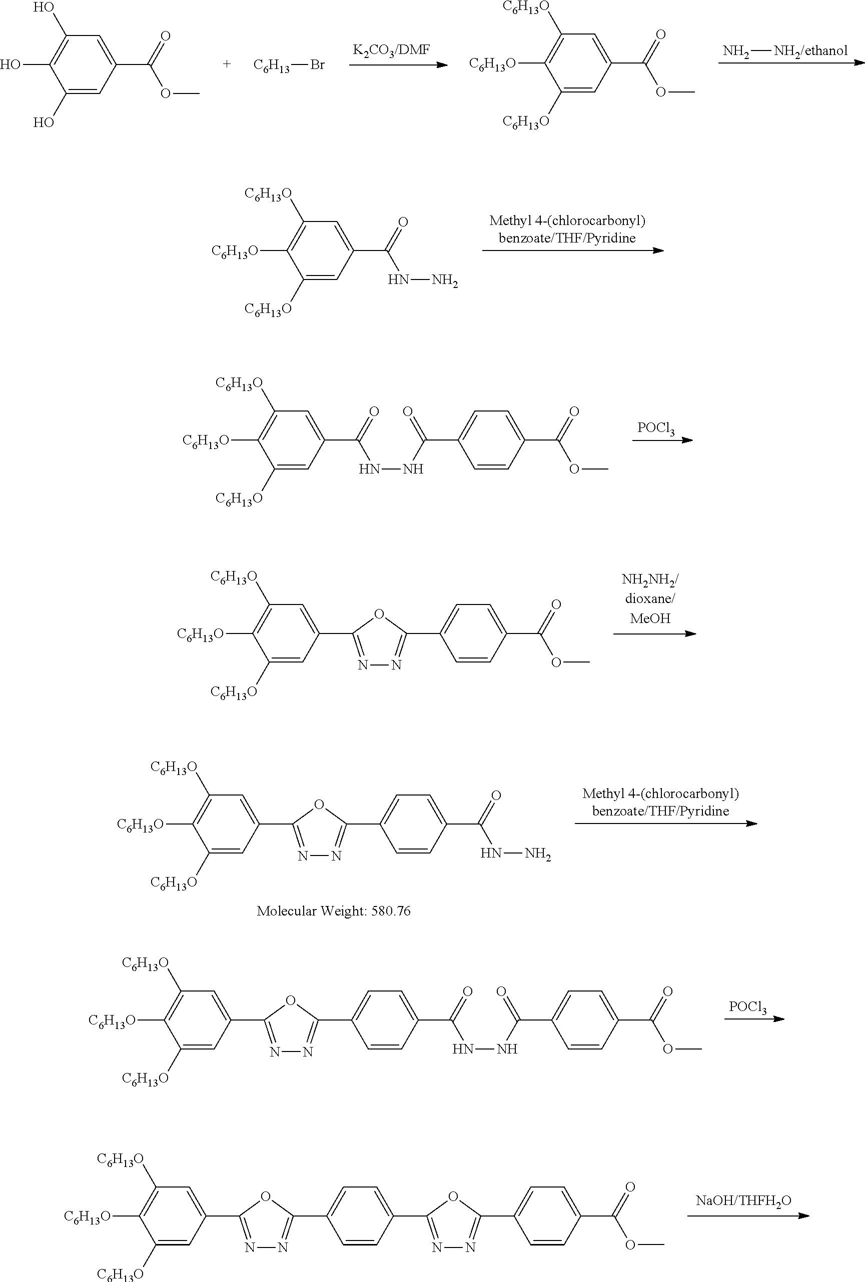

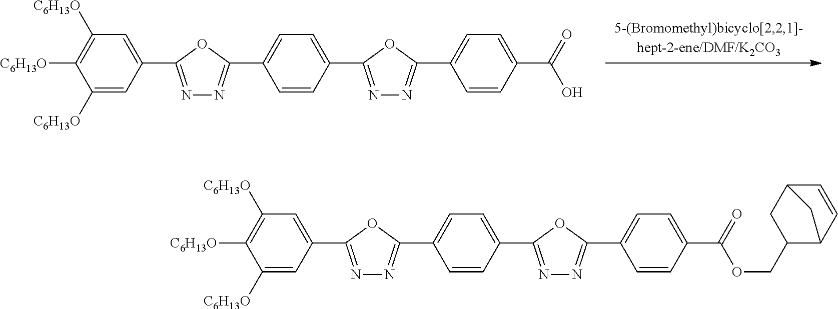

### Step 1: Methyl 3,4,5-Tris(hexanyloxy)benzoate YZ-2-37

A 250 ml round-bottom flask equipped with a Teflon-coated magnetic stirring bar was charged with 150 ml of DMF and 60.0 g (363.48 mmol) of 1-bromohexane. The mixture was sparged with nitrogen, and the 60.0 g of anhydrous K 2CO₃ and 20 g (108.61 mmol) of methyl 3,4,5-trihydroxybenzoate 1 were added as N₂ sparging was continued. The mixture was heated at 80° C. for 24 h with stirring under a N₂ atmosphere. The re action was judged complete by TLC analysis. The reaction mixture was cooled to room temperature. Water (700 ml) was added, and the product was extracted with ether. The organic phase was washed with water. The organic phase was separated and dried over MgSO₄. The solvent was evaporated, and then crude product was pass through a column of silica gel using Hexane: ethyl acetate (9.5:0.5) as eluent. The product was obtained a s yellow liquid in 44.4 g (93.6%).

¹H-NMR (500 MHz, CDCl₃) δ: 7.27 (s, 2H), 4.01 (m, 6H, 3×OCH2), 3.89 (s, 3H, COOCH₃), 1.82 (m, 4H, 2×CH₂), 1.75 (m, 2H, CH₂), 1.48 (m, 6H, 3×CH₂), 1.22 (m, 12H, 6×CH₂), 0.90 (m, 9H, 3×CH₃) ppm. ¹³C-NMR (100 MHz, CDCl₃) δ 166.90, 152.77, 142.26, 124.60, 107.87, 73.43, 69.09, 52.06, 31.69, 31.52, 30.23, 29.21, 25.74, 25.71, 25.67, 22.65, 22.59, 14.00 ppm.

### Step 2: 3,4,5-Tris(hexanyloxy)benzoichydrazide YZ-2-85

A mixture of 25.0 g of methyl 3,5-bis(hexanyloxy)benzoate (57.26 mmol) and an excess amount of hydrazine monohydrate (50.0 ml) was dissolved in ethanol (200 ml), and then the mixture was heated at 80° C. for 24 h. After the reaction was finished, water (200 ml) was poured into the reaction mixture and the product was precipitated. The white solid was collect ed and dried under vacuum. The pure white solid product was obtained by recrystallization from ethanol/water. The product yield was 23.7 g (94.8%).

¹H-NMR (500 MHz, CDCl₃) δ: 7.85 (s, 1H, NH), 6.97 (s, 2H), 3.97 (m, 8H, 3×OCH₂ and NH₂), 1.79 (m, 6H, 3×CH₂), 1.46 (m, 6H, 3×CH₂), 1.32 (m, 12H, 6×CH₂), 0.90 (t, 9H, 3×CH₃, J=7.0 Hz) ppm. ¹³C-NMR (126 MHz, CDCl₃) δ: 168.65, 153.08, 141.21, 127.36, 73.43, 69.17, 31.65, 31.48, 30.17, 29.20, 25.67, 25.63, 22.60, 22.54, 14.00, 13.96 ppm. Anal. calculated for C₂₅H₄₄N₂O₄ (436.63): C, 68.77; H, 10.16; N, 6.42. Found: C, 68.40; H, 9.99; N, 6.35.

### Step 3: Methyl 4-(2-(3,4,5-tris(hexyloxy)benzoyl)hydrazinecarbonyl)benzoate (YZ-2-73′)

To a solution of 3,4,5-Tris(hexanyloxy)benzoichydrazide (11.0 g, 25.19 mmol) in THF (100.0 ml) was added methyl 4-(chlorocarbonyl)benzoate (5.0 g, 25.18 mmol) at 0° C. The reaction was kept at 0° C. for 2 h and then at room temperature for 6 h. Pyridine (10.0 ml) was added. After 20 min of pyr. addition. Water (200.0 ml) was added into reaction mixture and crude product as a solid was collected. After dry, product was obtained in 14.7 g (97.5%) yield.

¹H-NMR (500 MHz, CDCl₃) δ: 10.35 (s, 1H, NH), 9.91 (s, 1H, NH), 8.02 (d, 2H, J=8.5 Hz), 7.88 (d, 2H, J=8.5 Hz), 7.07 (s, 2H), 3.98 (t, 2H, OCH₂, J=6.5 Hz), 3.93 (s, 3H, OCH₃), 3.91 (t, 4H, 2×OCH₂, J=6.5 Hz), 1.76 (m, 6H, 3×Ch₂), 1.47 (m, 6H, 3×CH₂), 1.30 (m, 12H, 6×CH₂), 0.88 (m, 9H, 3×CH₃) ppm ¹³C-NMR (126 MHz, CDCl₃) δ: 166.03, 165.60, 164.48, 153.11 , 141.77, 134.88, 133.30, 129.72, 127.40, 125.46, 105.63, 73.46, 69.09, 52.43, 31.71, 31.53, 30.24, 29.24, 25.69, 22.66, 22.58, 14.06, 13.99 ppm.

### Step 4: Methyl 4-(5-(3,4,5-tris(hexyloxy)phenyl)-1,3,4-oxadiazol-2-yl)benzoate (YZ-2-75′)

Methyl 4-(2-(3,4,5-tris(hexyloxy)benzoyl)hydrazinecarbonyl)benzoate (14.0 g, 23.38 mmol) was added to POCl₃ (60.0 ml). The reaction was heated to 80° C., and kept at this temperature for 4 h. After cooling, the reaction mixture was slowly added to ice water (1500.0 ml). The crude product was collected as yellow solid, and purified by silica gel column using ethyl acetate/hexane (2:8) as eluent. Pure product was obtained in 12.1 g (89.1%).

¹H-NMR (500 MHz, CDCl₃) δ: 8.23 (d, 2H, J=8.5 Hz), 8.20 (d, 2H, J=8.5 Hz), 7.33 (s, 2H), 4.09 (t, 4H, 2×OCH₂, J=6.5 Hz), 4.05 (t, 2H, OCH₂, J=6.5 Hz), 3.97 (s, 3H, OCH₃), 1.86 (m, 4H, 2×CH₂), 1.77 (m, 2H, CH₂), 1.52 (m, 6H, 3×CH₂), 1.37 (m, 12H, 6×CH₂), 0.92 (m, 9H, 3×CH₃) ppm. ¹³C-NMR (126 MHz, CDCl₃) δ: 166.14, 165.21, 163.61, 153.60, 141.52, 132.67, 130.22, 127.73, 126.78, 118.14, 105.44, 73.62, 69.36, 52.49, 31.70, 31.53, 30.26, 29.24, 25.73, 25.70, 22.68, 22.62, 14.08, 14.03 ppm.

### Step 5: 4-(5-(3,4,5-tris(hexyloxy)phenyl)-1,3,4-oxadiazol-2-yl)benzohydrazide (YZ-2-83′)

To a solution of Methyl 4-(5-(3,4,5-tris(hexyloxy)phenyl)-1,3,4-oxadiazol-2-yl)benzoate (5.6, 9.64 mmol) in MeOH/p-dioxane (60.0 ml : 60.0 ml) at 80° C. was added NH₂NH₂H₂O (10.0 g, 199.76 mmol). The reaction was kept at 80° C. for 14 h. After cooling, water (20 ml) was added. The product as white solid was collected by filtration. After dry, the product was obtained in 5.1 g (91.1%).

¹H-NMR (500 MHz, CDCl₃) δ: 8.19 (d, 2H, J=8.5 Hz), 7.91 (d, 2H, J=8.5 Hz), 7.80 (s, 1H, NH), 7.30 (s, 2H), 4.07 (m, 8H, 3×OCH₂ and NH₂), 1.85 (m, 4H, 2×CH₂), 1.77 (m, 2H, CH₂), 151 (m, 6H, 3×CH₂), 1.36 (m, 12H, 6×CH₂), 0.91 (9H, 3×CH₃) ppm. ¹³C-NMR (126 MHz, CDCl₃) δ: 167.58, 165.15, 163.45, 153.57, 141.46, 135.22, 127.61, 127.08, 126.85, 118.07 , 105.35, 73.62, 69.33, 31.69, 31.52, 30.23, 29.23, 25.71, 25.67, 22.65, 22.59, 14.06, 14.02 ppm.

### Step 6 : Methyl 4-(2-(4-(5-(3,4,5-tris(hexyloxy)phenyl)-1,3,4-oxadiazol-2-yl)benzoyl)-hydrazinecarbonyl)benzoate

To a solution of 4-(5-(3,4,5-tris(hexyloxy)phenyl)-1,3,4-oxadiazol-2-yl)benzohydrazide (4.0 g, 6.89 mmol) in THF (100.0 ml) was added 4-(chlorocarbonyl)benzoate (1.4 g, 7.05 mmol) at 0° C. The reaction was kept at 0° C. for 2 h and then at room temperature for 6 h. Pyridine (10.0 ml) was added. After 20 min of pyridine addition, water (200.0 ml) was added into reaction mixture. The crude product as a white solid was collected. After dry under vacuum, product was obtained in 4.8 g (92.3%) yield.

¹H-NMR (400 MHz, CDCl₃) δ: 9.91 (d, 1H, NH, J=4.4 Hz), 9.83 (d, 1H, NH, J=4.4 Hz), 8.20 (d, 2H, J=8.0 Hz), 8.10 (d, 2H, J=8.0 Hz), 8.01 (d, 2H, J=8.4 Hz), 7.93 (d, 2H, J=8.4 Hz), 7.31 (s, 2H), 4.05 (m, 6H, 3×OCH₂), 3.95 (s, 3H, OCH₃), 1.86-1.73 (m, 6H, 3×CH₂), 1.51 (m, 6H, 3×CH₂), 1.36 (m, 12H, 6×CH₂), 0.91 (m, 9H, 3×CH₃) ppm. ¹³C-NMR (100 MHz, CDCl₃) δ: 167.58, 165.15, 163.45, 153.57, 141.46, 135.22, 127.61, 127.08, 126.85, 118.07, 105.35, 73.62, 69.33, 31.69, 31.52, 30.23, 29.23, 25.71, 25.67, 22.65, 22.59, 14.06, 14.02 ppm.

### Step 7

Methyl 4-(5-(4-(5-(3,4,5-tris(hexyloxy)phenyl -1,3,4-oxadiazol-2-yl)phenyl)-1,3,4-oxadiazol-2-yl)benzoate:Methyl 4-(2-(4-(5-(3,4,5-tris(hexyloxy)phenyl)-1,3,4-oxadiazol-2-yl)benzoyl)hydrazinecarbonyl)benzoate (4.7 g, 6.32 mmol) was added to POCl₃ (60.0 ml). The reaction was heated to 80° C., and kept at this temperature for 4 h. After cooling, the reaction mixture was slowly added to ice water (400.0 ml). The crude product was collected as yellow solid, and purified by silica gel column using ethyl acetate/hexane (2:8) as eluent. Pure product was obtained in 4.12 g (89.8%).

¹H-NMR (400 MHz, CDCl₃): δ 8.33 (s, 4H), 8.25 (d, 2H, J=8.4 Hz), 8.23 (d, 2H, J=8.4 Hz), 7.34 (s, 2H), 4.08 (m, 6H, 3×OCH₂), 3.98 (s, 3H, OCH₃), 1.86-1.73 (m, 6H, 3×CH₂), 1.51 (m, 6H, 3×CH₂), 1.37 (m, 12H, 6×CH₂), 0.92 (m, 9H, 3×CH₃) ppm. ¹³C-NMR (100 MHz, CDCl₃) δ: 166.01, 165.24, 164.25, 164.16, 163.41, 153.63, 141.62, 133.06, 130.33, 127.58, 127.50, 127.32, 126.95, 126.90, 126.14, 118.06, 105.48, 73.62, 69.39, 52.53, 31.71, 31.54, 30.26, 29.25, 25.73, 25.69, 22.66, 22.60, 14.07, 14.02 ppm.

### Step 8: 4-(5-(4-(5-(3,4,5-tris(Hexyloxy)phenyl)-1,3,4-oxadiazol-2-yl)phenyl)-1,3,4-oxadiazol-2-yl)benzoic acid (YZ-I-177)

Methyl 4-(5-(4-(5-(3,4,5-tris(hexyloxy)phenyl -1,3,4-oxadiazol-2-yl)phenyl)-1,3,4-oxadiazol-2-yl)benzoate (4.0 g, 5.52 mmol) was taken into THF (180.0 ml) and methanol (60.0 ml). After the starting material was dissolved in THF/methanol, NaOH (6.0 g in 3.0 ml of water) was added into this solution mixture at room temperature. During addition of NaOH solution, the yellow solid was appeared. The reaction was kept at room temperature for 25 h. HCl (200.0 ml, 2 M) was added. During addition of HCl, yellow solid was disappeared. More HCl solution was added, the yellow solid product was appeared. The yellow solid product was collected by filtration. After drying under vacuum, the product was obtained in 3.6 g (92.3%) yield. This product could be used for next step without any future purification.

### Step 9: Bicyclo[2,2,1]hept-5en-2-ylmethyl 4-(5-(4-(5-(3,4,5-tris(hexyloxy)phenyl)-1,3,4-oxadiazol-2-yl)phenyl)-1,3,4-oxadiazol-2-yl)benzoate (YZ-I-179)

To a solution of 4-(5-(4-(5-(3,4,5-tris(Hexyloxy)phenyl)-1,3,4-oxadiazol-2-yl)phenyl)-1,3,4-oxadiazol-2-yl)benzoic acid (1.0 g, 1.41 mmol) and 5-(bromomethyl)bicycle[2,2,1]hept-2-ene (0.6 g, 3.21 mmol) in DMF (25.0 ml), K₂CO₃ (2.0 g, 14.47 mmol) was added at room temperature. The reaction was carried out at 100° C. for 48 hours. After cooling down to room temperature, the water (150.0 ml) was added into the reaction mixture. A whit e solid precipitate was collected by filtration and dried under vacuum. The crude product was purified by silica gel column chromatography, eluting with dichloromethane and ethyl acetate in a 9:1 ratio. After evaporating the solvent, the white solid was recrystallized from dichloromethane/methanol and finally dried under vacuum. A pure product was obtained as a white solid in 1.06 g (93.0%) yield.

¹H NMR (400 MHz, CDCl₃) δ: 8.32 (s, 4H), 8.21 (m, 4H), 7.34 (s, 2H), 6.24-6.02 (m, 2H, C═C—H, endo and exo), 4.48-3.96 (m, 14H), 2.90 (m, br), 2.85 (s, br), 2.59 (m, br), 1.97-1.75 (m, 7H), 1.54 (m, 7H), 1.42 (s, 14H), 0.94 (m, 9H), 0.68 (m) ppm. ¹³C NMR (100 MHz, CDCl₃) δ: 165.24, 165.03, 164.07, 163.94, 163.20, 153.45, 141.49, 137.66, 136.91, 135.9 9, 133.35, 131.95, 130.16, 127.45, 127.37, 127.10, 126.80, 126.05, 117.97, 105.45, 73.62, 69.57, 69.42, 68.90, 49.49, 45.08, 44.05, 43.79, 42.30, 41.70, 38.14, 37.95, 31.81, 31.64, 30.37, 29.72, 29.37, 29.08, 25.85, 25.82, 22.79, 22.73, 14.22, 14.17 ppm. MS (m/z): [M]⁺ calcd for C₄₉H₆₀N₄O₇817.5, found 817.6. Anal. Calcd for C₄₉H₆₀N₄O₇: C, 72.03; H, 7.40; N, 6.86. Found: C, 71.91; H, 7.37; N, 6.79.

### Preparative Example 10

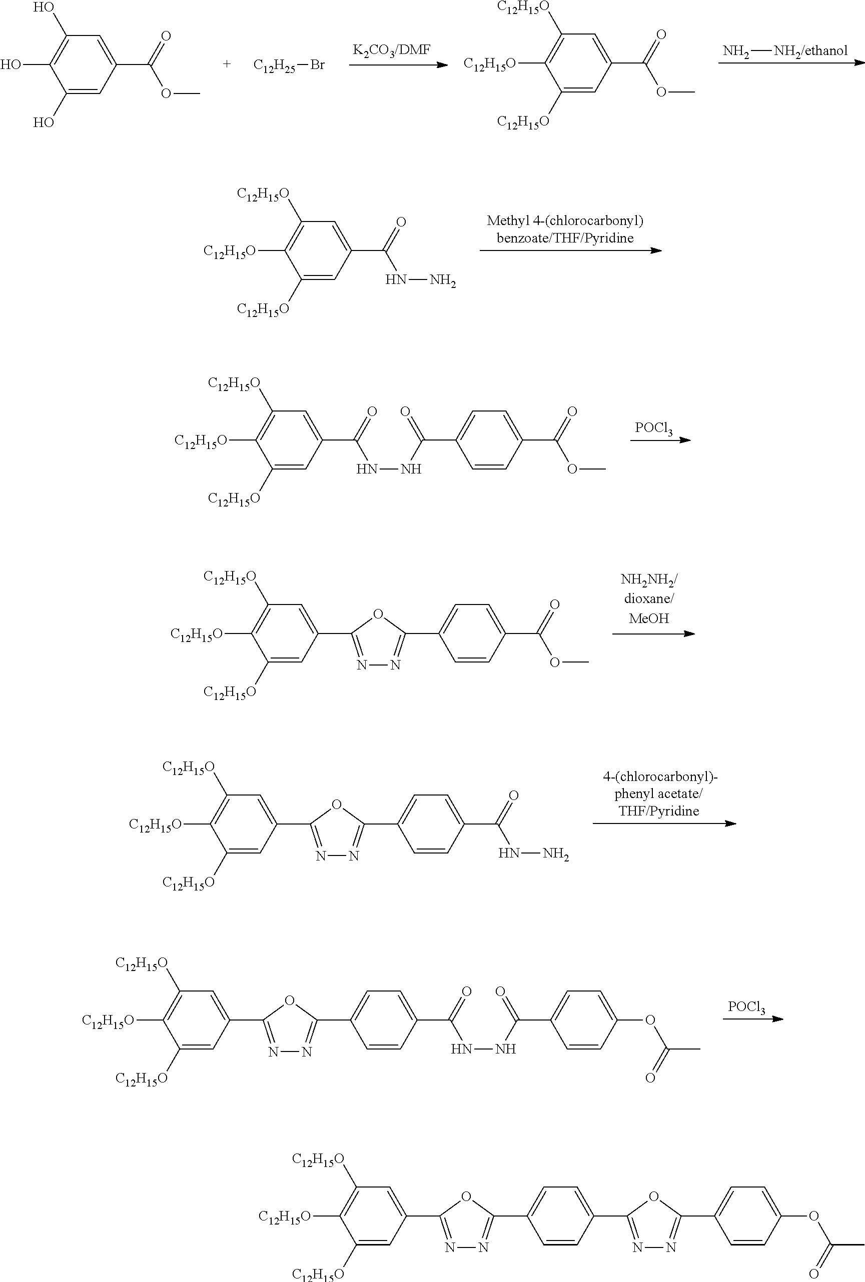

### Step 1: Methyl 3,4,5-Tris(dodecanyloxy)benzoate YZ-2-43

A 250 ml round-bottom flask equipped with a Teflon-coated magnetic stirring bar was charged with 200 ml of DMF and 80.0 g (320.99 mmol) of 1-bromododecane. The mixture was sparged with nitrogen, and the 60.0 g of anhydrous K₂CO₃ and 18.0 g (97.75 mmol) of methyl 3,4,5-trihydroxybenzoate 1 were added as N₂ sparging was continued. The mixture was heated at 80° C. for 24 h with stirring under a N₂ atmosphere. The reaction was judged complete by TLC analysis. The reaction mixture was cooled to room temperature. Water (700 ml) was added, and the product was extracted with ether. The organic phase was washed with water. The organic phase was separated and dried over MgSO₄. The solvent was evaporated, and then crude product was pass through a column of silica gel using Hexane : ethyl acetate (9.5:0.5) as eluent. The product was obtained as yellow liquid in 64.6 g (95.9%). ¹H-NMR (CDCl₃, TMS, 500 MHz): δ: 7.25 (s, 2 Hₐᵣₒₘ), 4.01 (m, 6H, 3×OCH₂), 3.89 (s, 3H, OCH₃), 1.81 (m, 4 h, 2×CH₂), 1.72 (m, 2H, CH₂), 1.47 (m, 6H, 3×CH₂), 1.26 (m, 48H, 24×CH₂), 0.88 (t, 9H, 3×CH₃, J=7.5 Hz) ppm.

¹³C-NMR (CDCl₃, 126 MHz) δ: 166.93, 152.77, 142.22, 124.60, 107.85, 73.45, 69.09, 52.10, 31.91, 30.30, 29.71, 29.68, 29.63, 29.56, 29.38, 29.35, 29.27, 26.06, 26.03, 22.69, 14.12 ppm.

### Step 2: 3,4,5-Tris(dodecanyloxy)benzoichydrazide YZ-2-59

A mixture of 20.0 g of methyl 3,4,5-bis(dodecanyloxy)benzoate (29.02 mmol) and an excess amount of hydrazine monohydrate (38.0 ml) was dissolved in ethanol (250 ml), and then the mixture was heated at 80° C. for 14 h. After the reaction was finished, water (280 ml) was poured into the reaction mixture and the product was precipitated. The white solid was collected and dried under vacuum. The pure white solid product was obtained by recrystallization from ethanol/water. The product yield was 19.1 g (95.5%).

¹H-NMR (CDCl₃, TMS, 500 MHz): δ: 7.61 (s, 1H, CONH), 6.95 (s, 2 Hₐᵣₒₘ), 3.98 (m, 8H, 3×OCH₂, NH₂), 1.79 (m, 4H, 2×CH₂), 1.73 (m, 2H, CH₂), 1.46 (m, 6H, 3×CH₂), 1.26 (m, 48H, 24×CH₂), 0.88 (t, 9H, 3×CH₃, J=7.0 Hz) 73.47, 69.23, 31.89, 30.25, 29.67, 29.62, 29.60, 29.54, 29.35, 29.33, 29.27, 26.03, 22.66, 14.08 ppm. Anal. calculated for C₄₃H₈₀N₂O₄ (689.11): C, 74.95;H, 11.70; N, 4.07. Found: C, 74.66;H, 11.81; N, 4.15.

### Step 3: Methyl 4-(2-(3,4,5-tris(dodecyloxy)benzoyl)hydrazinecarbonyl)benzoate (YZ-2-73′)

To a solution of 3,4,5-Tris(dodecanyloxy)benzoichydrazide (10.0 g, 14.51 mmol) in THF (100.0 ml) was added methyl 4-(chlorocarbonyl)benzoate (5.0 g, 25.18 mmol) at 0° C. The reaction was kept at 0° C. for 2 h and then at room temperature for 6 h. Pyridine (10.0 ml) was added. After 20 min of pyridine addition, water (200.0 ml) was added in to reaction mixture and crude product as a solid was collected. After dry, product was obtained in 14.7 g (97.5%) yield.

¹H-NMR (500 MHz, CDCl₃) δ: 10.35 (s, 1H, NH), 9.91 (s, 1H, NH), 8.02 (d, 2H, J=8.5 Hz), 7.88 (d, 2H, J=8.5 Hz), 7.07 (s, 2H), 3.98 (t, 2H, OCH₂, J=6.5 Hz), 3.93 (s, 3H, OCH₃), 3.91 (t, 4H, 2×OCH₂, J=6.5 Hz), 1.76 (m, 6H, 3×CH₂), 1.47 (m, 6H, 3×CH₂), 1.30 (m, 12H, 6×CH₂), 0.88 (m, 9H, 3×CH₃) ppm. ¹³C-NMR (126 MHz, CDCl₃) δ: 166.03, 165.60, 164.48, 153.11, 141. 77, 134.88, 133.30, 129.72, 127.40, 125.46, 105.63, 73.46, 69.09, 52.43, 31.71, 31.53, 30.24, 29.24, 25.6 ppm.

### Step 4: Methyl 4-(5-(3,4,5-tris(dodecyloxy)phenyl)-1,3,4-oxadiazol-2-yl)benzoate

Methyl 4-(2-(3,4,5-tris(dodecyloxy)benzoyl)hydrazinecarbonyl)benzoate (10.0 g, 11.75 mmol) was added to POCl₃ (60.0 ml). The reaction was heated to 80° C., and kept at this temperature for 5 h. After cooling, the reaction mixture was slowly added to ice water (800.0 ml). The crude product was collected as yellow solid, and purified by silica gel column using ethyl acetate/hexane (2:8) as eluent. Pure product was obtained in 7.8 g (79.6%).

¹H-NMR (400 MHz, CDCl₃) δ: 8.21 (ss, 4H), 7.33 (s, 2H), 4.07 (m, 6H, 3×OCH₂), 3.98 (s, 3H, OCH₃), 1.90-1.73 (m, 6H, 3×CH₂), 1.50 (m, 6H, 3×CH₂), 1.27 (m, 48H, 24×CH₂), 0.88 (m, 9H, 3×CH₃) ppm.

### Step 5: 4-(5-(3,4,5-tris(dodecyloxy)phenyl)-1,3,4-oxadiazol-2-yl)benzohydrazide

To a solution of Methyl 4-(5-(3,4,5-tris(dodecyloxy)phenyl)-1,3,4-oxadiazol-2-yl)benzoate (6.0, 7.20 mmol) in MeOH/dopxame (60.0 mL 100 ml) at 80° C. was added hydrazine hydrate (10.0 g, 199.76 mmol). The reaction was kept at 80° C. for 24 h. After cooling, water (200.0 ml) was added. The product as white solid was collected by filtration. After dry, the product was obtained in 5.6 g (93.3%).

¹H-NMR (400 MHz, CDCl₃) δ: 8.21 (d, 2H, J=8.0 Hz), 7.92 (d, 2H, J=8.0 Hz), 7.59 (s, 1H, NH), 7.31 (s, 2H), 4.19 (s, br, 2H, NH₂), 4.07-4.03 (m, 6H, 3×OCH₂), 1.89-1.74 (m, 6H, 3×CH₂), 151 (m, 6H, 3×CH₂), 1.27 (m, 48H, 24×CH₂), 0.88 (9H, 3×CH₃) ppm.

### Step 6 : 4-(2-(4-(5-(3,4,5-Tris(dodecyloxy)phenyl)-1,3,4-oxadiazol-2-yl)benzoyl)-hydrazinecarbonyl)phenyl acetate (YZ-I-211)

To a solution of 4-(5-(3,4,5-tris(dodecyloxy)phenyl)-1,3,4-oxadiazol-2-yl)benzohydrazide (2.5 g, 3.00 mmol) in THF (100.0 ml) was added 4-(chlorocarbonyl)phenyl acetate (0.7 g, 3.52 mmol) at room temperature. The reaction was kept at room temperature for 21 h. Pyridine (6.0 ml) was added into reaction mixture. The reaction mixture was stirred for another 60 min. Water (300.0 ml) was added into reaction mixture. The crude product as a white solid was collected. After dry under vacuum, product was obtained in 2.7 g (90.0%) yield.

¹H-NMR (400 MHz, CDCl₃) δ: 9.69 (d, 1H, NH, J=4.4 Hz), 9.54 (d, 1H, NH, J=4.4 Hz), 8.20 (d, 2H, J=8.0 Hz), 8.03 (d, 2H, J=8.8 Hz), 7.91 (d, 2H, J=8.8 Hz), 7.32 (s, 2H), 7.20 (d, 2H, J=8.8 Hz), 4.07 (m, 6H, 3×OCH₂), 2.33 (s 3H, CH₃), 1.90-1.73 (m, 6H, 3×CH₂), 1.50 (m, 6H, 3×CH₂), 1.27 (m, 48H, 24×CH₂), 0.88 (m, 9H, 3×CH₃) ppm.

### Step 7

4-(5-(4-(5-(3,4,5-Tris(dodecyloxy)phenyl)-1,3,4-oxadiazol-2-yl)phenyl)-1,3,4-oxadiazol-2-yl)phenyl acetate (YZ-I-219): 4-(2-(4-(5-(3,4,5-Tris(dodecyloxy)phenyl)-1,3,4-oxadiazol-2-yl)benzoyOhydrazinecarbonyl)phenyl acetate (2.5 g, 2.51 mmol) was added to POCl₃ (35.0 ml). The reaction was heated to 100° C., and kept at this temperature for 5 h. After cooling, the reaction mixture was slowly added to ice water (400.0 ml). The crude product was collected as yellow solid, and purified by silica gel column using dichloromethane/ethyl acetate (9:1) as eluent. Pure product was obtained in 1.23 g (50.2%).

¹H NMR (400 MHz, CDCl₃) δ: 8.32 (s, 4H), 8.21 (d, 2H, J=8.8 Hz), 7.34 (s, 2H), 7.32 (d, 2H, J=8.8 Hz), 4.11-4.04 (m, 6H, 3×CH₂), 2.36 (s, 3H, CH3), 1.90-1.75 (m, 6H, 3×CH₂), 1.504 (m, 6H, 3×CH₂), 1.27 (m, 48H, 24×CH₂), 0.88 (m, 9H, 3×CH₃), 0.68 (m) ppm. ¹³C NMR (100 MHz, CDCl₃) δ: 168.89, 165.22, 164.37, 163.77, 163.47, 153.63, 153.45, 141.57, 128.44, 127.48, 126.70, 126.36, 122.57, 121.17, 118.09, 105.46, 73.64, 69.38, 31.09, 30.32, 29.73, 29.69, 29.65, 29.63, 29.57, 29.40, 29.35, 29.30, 26.08, 22.68, 21.16, 14.11 ppm. MS-EI (m/z): [M]⁺ calcd for C₆₀H₈₈N₄O₇ 976.7, found 976.5.

### Preparative Example 11

### Synthesis of SKP-I-ODZ-31

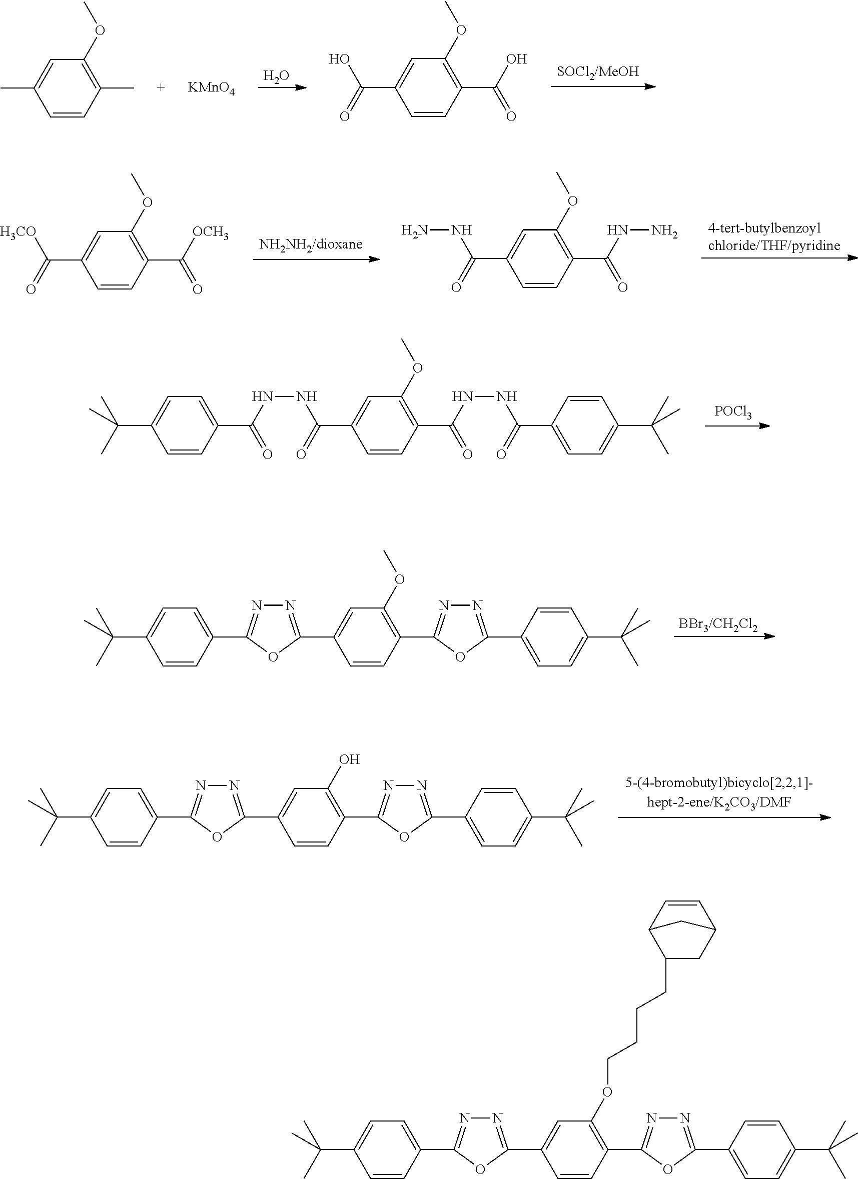

### Step 1: 2-Methoxyterephthalic acid (SKP-I-ODZ-20)

2,5-Dimethylanisole (30.0 g, 220.5 mmol), potassium permanganate (120 g, 760 mmol) and 1000 mL water was taken into a round bottom flask and reflux for 6 hours. After cooling down to room temperature the reaction was poured into 500 mL of ice cold ethanol and then stirred for ½ an hours. The mixture was filtered, concentrated and then acidified with hydrochloric acid. The white precipitate formed was collected by filtration and dried. Yield=19.7 g (46%). Reported yield is 50%.

¹H NMR (400 MHz, Acetone-d₆) δ: 11.40 (s, br, 2H), 7.91 (d, 1H, J=8.0 Hz), 7.73 (d, 1H, J=3.2 Hz), 7.69 (dd, 1H, J₁=2.4 Hz, J₂=7.6 Hz), 4.04 (s, 3H) ppm.

### Step 2: Dimethyl 2-methoxyterephthalate (SKP-I-ODZ-23)

2-Methoxyterephthalic acid (10.0 g, 51.02 mmol) was dissolved in 400 mL of methanol. 50 mL of SOCl₂ was added dropwise using an addition funnel The reaction was stirred at room temperature for 15 hours and then poured into excess of water. White slurry was obtained and the mixture was neutralized with 30% Na₂CO₃ solution, filtered and then washed with plenty of water. After drying under vacuum 8.80 g (77%) white solid was obtained. ¹H NMR (400 MHz, CDCl₃) δ: 7.80 (d, 1H, J=8.4 Hz), 7.64 (m, 2H), 3.96 (s, 3H), 3.94 (s, 3H), 3.91 (s, 3H) ppm.

### Step 3: 2-Methoxyterephthalohydrazide (SKP-I-ODZ-24)

Dimethyl 2-methoxyterephthalate (5.0 g, 22.3 mmol) was dissolved in 25 mL of p-dioxane followed by addition of 8.88 mL of hydrazine monohydrate. The reaction was stirred at 85° C. for 7 hours. After cooled down to room temperature 300 mL of water was added. White solid formed was filtered and washed with water and dried. Yield obtained 4.6 g (92%). ¹H NMR (400 MHz, DMSO-d₆) δ: 9.89 (s, br, 1H), 9.31 (s, br, 1H), 7.64 (s, br, 1H), 7.47 (m, 2H), 4.54 (s, 2H), 3.88 (s, 2H) ppm.

### Step 4: N′¹, N′⁴-Bis(4-tert-butylbenzoyl)-2-methoxyterephthalohydrazide (SKP-I-ODZ-25)

2-Methoxyterephthalohydrazide (4.0 g, 17.86 mmol) was dissolved in 125 mL of dry tetrahydrofuran and then 7.02 mL (35.8 mmol) of 4-tertbutylbenzoyl chloride was added dropwise. The reaction was stirred 7 hours at room temperature and after that 10 mL of pyridine was added and stirred for another ½ an hour before pouring it into 500 mL of water. White precipitate obtained was collected by filtration and washed with plenty of water. After drying under vacuum for 12 hours 8.5 g (87%) of white solid was obtained. MS-EI (m/z): [M]⁺ calcd for C₃₁H₃₆N₄O₅O₅ 544, found 544. ¹H NMR (400 MHz, DMSO-d₆) δ: 10.65 (s, 1H), 10.58 (s, 1H), 10.49 (s, 1H), 10.15 (s, 1H), 8.04 (d, 1H, J=8.4 Hz), 7.79-7.88 (m, 5H), 7.49-7.64 (m, 5H), 3.97 (s, 3H), 1.31 (s, 18H) ppm. ¹³C NMR (75.5 MHz, DMSO-d₆) δ: 166.43, 165.93, 165.70, 164.96, 157.54, 155.49, 155.38 , 145.19, 144.05, 136.81, 131.13, 130.43, 128.07, 127.36, 126.03, 125.96, 125.85, 120.29, 111.61, 56.78, 55.62, 35.41, 31.61 ppm. Anal. Calcd for C₃₁H₃₆N₄O₅: C, 68.36;H, 6.66; 10.29. Found: C, 65.16; H, 6.62; N, 9.78.

### Step 5: 5,5′-(2-methoxy-1,4-phenylene)bis(2-(4-tert-butylphenyl)1,3,4-oxadiazole) (SKP-I-ODZ-27)

N′¹,N′⁴-Bis(4-tert-butylbenzoyl)-2-methoxyterephthalohydrazide (2.0 g, 3.68 mmol) was suspended in 75 mL of POCl₃ and the reaction was refluxed at 96° C. for 8 hours. During the reaction the solid SKP -I-ODZ-25 were completely dissolved in POCl₃. After cooling down to room temperature the mixture was poured into 250 mL of ice-water mixture. The light yellow solid formed was collected by filtration and dried under vacuum. The yield of the reaction is 1.75 g (94%). MS -EI (m/z): [M]⁺ calcd for C₃₁H₃₂N₄O₃ 508, found 508. ¹H NMR (400 MHz, CDCl₃) δ: 8.22 (d, 1H, J=8.0 Hz), 8.08-8.11 (m, 4H), 7.89 (s, 1H), 7.83 (dd, 1H, J₁=1.6 Hz, J₂=8.0 Hz, 1H), 7.56-7.59 (m, 4H), 4.14 (s, 3H), 1.39 (s, 9H), 1.38 (s, 9H) ppm.

¹³C NMR (75.5 MHz, CDCl₃) δ: 165.15, 164.78, 163.50, 162.32, 158.05, 155.70, 155.41, 131.03, 127.77, 126.88, 126.86, 126.10, 126.02, 120.9 4, 120.67, 118.95, 115.90, 109.99, 56.44, 35.09, 35.07, 31.07 ppm. Anal. Calcd for C₃₁H₃₂N₄O₃: C, 73.21; H, 6.34; N, 11.02. Found: C, 72.53; H, 6.49; N, 10.91.

### Step 6 : 2,5-Bis(5-(4-tert-butylphenyl)-1,3,4-oxadiazol-2-yl)phenol (SKP-I-ODZ-30)

5,5′-(2-methoxy-1,4-phenylene)bis(2-(4-tert-butylphenyl)1,3,4-oxadiazole) (1.0 g, 1.97 mmol) was dissolved in 30 mL of dry dichloromethane. Boron tribromide (2.6 mL, 27.5 mmol) 1 (M) solution in dichloromethane was added drop-wise by using a syringe at −70° C. After ½ hours reaction was taken to room temperature and stirred overnight. Then the reaction mixture was poured into ice-water and neutralized with sodium carbonate solution. Dichloromethane was evaporated under reduced pressure and the pale yellow solid was collected by filtration and dried in vacuum. The yield of the reaction is 0.9 g (92%). MS-EI (m/z): [M]⁺ calcd for C₃₀H₃₀N₄O₃ 494, found 494 ¹H NMR (400 MHz, CDCl₃) δ: 10.48 (br., 1H), 8.08-8.11 (m, 4H), 8.03 (d, 1H, J=12 Hz), 7.89 (s, 1H), 7.88 (d, 1H, J=8.0 Hz), 7.57-7.61 (m, 4H), 1.39 (s, 9H), 1.38 (s, 9H) ppm. ¹³C NMR (100 MHz, CDCl₃) δ:164.85, 163.56, 163.10, 163.01, 157.52, 155.97, 155.45, 128.04, 127.07, 126.85, 126.71, 126.12, 125.98, 120.57, 119.92, 118.07, 115.55, 110.44, 35.26, 35.2 0, 31.18, 31.17 ppm. Anal. Calcd for C₃₀H₃₀N₄O₃: C, 72.85; H, 6.11; N, 11.33. Found: C, 71.46; H, 6.02; N, 11.05.

### Step 7: 5,5′-(2-(4-(bicycle[2,21]hept-5-en-2-yl)butoxy)-1,4-phenylene)bis(2-(4-tert-butylphenyl)-1,3,4-oxadiazole) (SKP-I-ODZ -31)

2,5-Bis(5-(4-tert-butylphenyl)-1,3,4-oxadiazol-2-yl)phenol (1.0 g, 2.02 mmol) was dissolved in 3 mL of dry dimethylforamide followed by addition of 0.345 g (2.5 mmol) of K₂CO₃. After stirring for a while 5-norbornene-2-butyl bromide (0.465 g, 2.03 mmol) was added. The reaction was stirred 15 hours at 80° C. After cooling down to room temperature the reaction was poured into 100 mL of water. The yellow solid formed was collected by filtration and the crude material was purified by column chromatography eluting with hexane and ethyl acetate in 2:1 ratio. After evaporating solvent the white solid was washed with methanol and finally dried under vacuum. The yield of the reaction is 0.75 g (58%). MS -EI (m/z): [M]⁺ calcd for C₄₁H₄₆N₄O₃ 642, found 642. ¹H NMR (400 MHz, CDCl₃) δ: 8.28 (d, J=8.0 Hz, 1H), 8.07-8.11 (m, 4H), 7.85 (s, 1H), 7.80 (d, J=8.0 Hz, 1H), 7.57 (t, J=8.0 Hz, 4H), 6.08 (m, 1H), 5.87 (m, 1H), 4.26 (t, J=6.4 Hz, 2H), 1.89-1.99 (m, 1H), 1.78-1.84 (m, 2H), 1.52-1.63 (m, 3H), 1.35-1.43 (m, 20H), 1.20 (m, 2H), 0.46-0.51 (m, 2H) ppm. ¹³C NMR (100 MHz, CDCl₃) δ: 164.89, 164.80, 163.38, 162.65, 157.24, 155.45, 155.06, 136.84, 132.09, 131.08, 127.58, 126.75, 126.59, 125.97, 125.90, 121.07, 120.63, 118.63, 115.99, 110.61, 69.31, 49.58, 45.46, 42.54, 38.75, 35.20, 35.16, 34.63, 32.46, 31.21, 31.19, 29.62, 25.38 ppm. Anal. Calcd for C₄₁H₄₆N₄O₃: C, 76.60; H, 7.21; N, 8.72. Found: C, 76.50; H, 7.20; N, 8.63.

### Preparative Example 12

### Synthesis of YZ-1-285

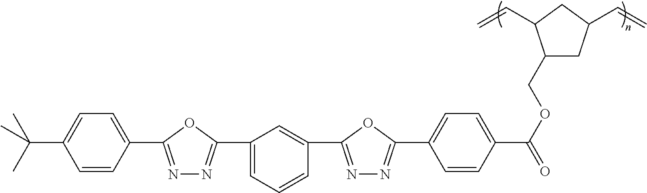

### Poly(bicyclo[2,2,1]hept-5-en-2-ylmethyl 4-(5-(3-(5-(4-tert-butylphenyl)-1,3,4-oxadiazol-2-yl)-phenyl)-1,3,4-oxadiazol-2-yl)benzoate) (YZ-I-285)

Bicyclo [2,2,1 ]hept-5-en-2-ylmethyl-4-(5-(3-(5-(4-tert-butylphenyl)-1,3,4-oxadiazol-2-yl)phenyl)-1,3,4-oxadiazol-2-yl)benzoate (0.50 g, 0.873 mmol), and a 1^{st} generation Grubbs catalyst (7.2 mg, 0.0088 mmol) were mixed well in CH₂Cl₂ (15.0 ml) at room temperature, under stirring in a glove box. The reaction was carried out at room temperature for 23 hours. The reaction vial was taken out from the glove box. Then, ethyl vinyl ether (2.0 ml) was added to the reaction mixture. The reaction mixture was stirred for 1 hour. A polymer solution was dropped into methanol (75.0 ml) to give a white polymer solid. The white solid product was collected by filtration. The reprecipitation procedure in dichloromethane/methanol was then repeated three times. After filtration and drying in a vacuum, the final product as a white solid was obtained in 0.30 g (60.0%) yield.

¹H NMR (CDCl₃) δ: 8.65 (m, br, 1H), 8.10 (m, br, 8H), 7.49 (m, br, 3H), 5.40 (s, br, 2H, 2×C═C—H), 4.13 (m, br, 2H, OCH₂), 3.25-1.00 (m, br, 7H), 1.33 (s, br, 9H, 3×CH₃) ppm. Anal. Calcd for C₃₅H₃₂N₄O₄: C, 73.41; H, 5.63; N, 9.78. Found: C, 72.77; H, 5.64; N, 9.60. GPC (THF): M_{w}=99000, Mₙ=40000, PDI=2.5.

### Preparative Example 13

### Synthesis of YZ-I-287

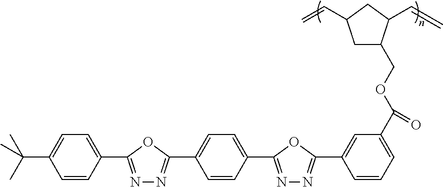

### Poly(bicyclo[2,21]hept-5-en-2-ylmethyl 3-(5-(4-(5-(4-tert-butylphenyl)-1,3,4-oxadiazol-2yl)-phenyl)-1,3,4-oxadiazol-2-yl)benzoate) (YZ-I-287)

Bicyclo [2,21 ]hept-5-en-2-ylmethyl 3-(5-(4-(5-(4-tert-butylphenyl)-1,3,4-oxadiazol-2-yl)phenyl)-1,3,4-oxadiazol-2-yl)benzoate (0.50 g, 0.873 mmol), and a 1^{st} generation Grubbs catalyst (7.2 mg, 0.0088 mmol) were mixed well in CH₂Cl₂ (12.0 ml)) at room temperature under stirring in a glove box. The reaction was carried out at room temperature for 23 hours. The reaction vial was taken out from the glove box. Then, ethyl vinyl ether (2.0 ml) was added to the reaction mixture. The reaction mixture was stirred for 30 minutes. A polymer dichloromethane solution was dropped into methanol (100.0 ml) to give a white polymer solid. The white solid product was collected by filtration. The reprecipitation procedure in dichloromethane/methanol was then repeated five times. After filtration and drying in a vacuum, the final product as a white solid was obtained in 0.40 g (80.0%) yield.

¹H NMR (CDCl₃) δ: 8.65 (s, br, 1H), 8.16 (m, br, 8H), 7.49 (m, br, 3H), 5.40 (s, br, 2H, 2×C═C—H), 4.13 (m, br, 2H, OCH₂), 3.25-1.00 (m, br, 7H), 1.33 (s, br, 9H, 3×CH₃) ppm. Anal. Calcd for C₃₅H₃₂N₄O₄: C, 73.41; H, 5.63; N, 9.78. Found: C, 72.82; H, 5.68; N, 9.64. GPC (THF): M_{w}=77000, Mₙ=29000, PDI =2.7.

### Preparative Example 14

### Synthesis of YZ-I-289

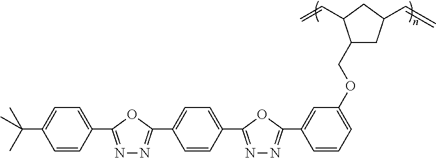

### Poly(2-(3-(Bicyclo[2,2,1]hept-5-en-2-ylmethoxy)phenyl)-5-(4-(5-(4-tert-butylphenyl)-1,3,4-oxadiazol-2-yl)phenyl)-1,3,4-oxadiazole) (YZ-I-289)

To a solution of 2-(3-(bicyclo-[2,2,1]hept-5-en-2-ylmethoxy)phenyl)-5-(4-(5-(4-tert-butylphenyl)-1,3,4-oxadiazol-2-yl)-phenyl)-1,3,4-oxadiazole (0.50 g, 0.920 mmol) in dichloromethane (10.0 ml), was added a 1^{st} generation Grubbs catalyst (7.5 mg, 0.0091 mmol) in CH₂Cl₂ (2.0 ml)) at room temperature, under stirring in a glove box. The reaction was carried out at room temperature for 22 hours. The reaction vial was taken out from the glove box. Then, ethyl vinyl ether (2.0 ml) was added to the reaction mixture. The reaction mixture was stirred for 30 minutes. A polymer solution was dropped into methanol (100.0 ml) to give a white polymer solid. The white solid product was collected by filtration. Next, the reprecipitation procedure in dichloromethane/methanol was repeated five times. After filtration and drying in a vacuum, the final product as a white solid in 0.42 g (84.0%) was obtained.

¹H NMR (CDCl₃) δ: 8.00 (m, br, 6H), 7.60-6.60 (m, br, 6H), 5.42 (m, br, 2H, 2×C═C—H), 3.85 (m, br, 2H, OCH2), 3.00 to 1.00 (m, br, 7H), 1.34 (s, 9H, 3×CH₃) ppm. Anal. Calcd for C₃₄H₃₂N₄O₃: C, 74.98; H, 5.92; N, 10.29. Found: C, 74.37; H, 5.89; N, 10.15. GPC (THF): M_{w}=113000, Mₙ=35000, PDI=3.2.

### Preparative Example 15

### Synthesis of YZ-I-291

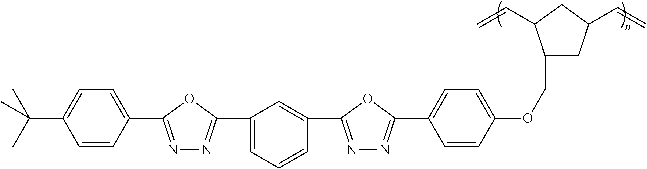

### Poly(2-(4-(bicyclo[2,2,1]hept-5-en-2-ylmethoxy)phenyl)-5-(3-(5-(4-tert-butylphenyl)-1,3,4-oxadiazol-2-yl)phenyl)-1,3,4-oxadiazole) (YZ-I-291)

To a solution of 2-(4-(bicyclo[2,2,1]hept-5-en-2-ylmethoxy)phenyl)-5-(3-(5-(4-tert-butylphenyl)-1,3,4-oxadiazol-2-yl)phenyl)-1,3,4-oxadiazole (0.50 g, 0.920 mmol) in dichloromethane (8.0 ml), was added a 1^{st} generation Grubbs catalyst (7.5 mg, 0.0091 mmol) in CH₂Cl₂ (2.0 ml)) at room temperature, under stirring in a glove box. The reaction was carried out at room temperature for 22 hours. The reaction vial was taken out from the glove box. Then, ethyl vinyl ether (2.0 ml) was added to the reaction mixture. The reaction mixture was stirred for 3 hours. A polymer solution was added to methanol (100.0 ml) to give a white polymer solid. The white solid product was collected by filtration. Then, the reprecipitation procedure in dichloromethane/methan of was repeated three times. After filtration and drying in a vacuum, the final product as a white solid in 0.41 g (82.0%) was obtained.

¹H NMR (CDCl₃) δ: 8.67 (m, br, 1H), 8.02 (m, br, 6H), 7.52 (m, br, 3H), 6.80 (m, 2H), 5.30 (m, br, 2H, 2×C═C—H), 3.85 (m, 2H, OCH₂), 3.25 to 1.00 (m, br, 7H), 1.35 (s, 9H, 3×CH₃) ppm. Anal. Calcd for C₃₄H₃₂N₄O₃: C, 74.98; H, 5.92; N, 10.29. Found: C, 74.37; H, 5.89; N, 10.15. GPC (THF): M_{w}=11900000, Mₙ=71000, PDI=166.7.

### Preparative Example 16

### Synthesis of YZ-I-293

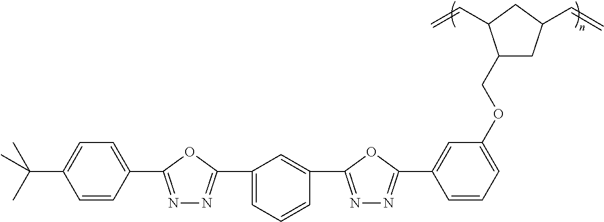

### Poly(2-(3-(bicyclo[2,2,1]hept-5-en-2-ylmethoxy)phenyl)-5-(3-(5-(4-tert-butylphenyl)-1,3,4-oxadiazol-2-yl)phenyl)-1,3,4-oxadiazole) (YZ-I-293)

To a solution of 2-(3-(bicyclo[2,2,1]-hept-5-en-2-ylmethoxy)phenyl)-5-(3-(5-(4-tert-butylphenyl)-1,3,4-oxadiazol-2-yl)phenyl)-1,3,4-oxadiazole (0.50 g, 0.920 mmol) in dichloromethane (8.0 ml), was added a 1^{st} generation Grubbs catalyst (7.5 mg, 0.0091 mmol) in CH₂Cl₂ (2.0 ml)) at room temperature, under stirring in a glove box. The reaction was carried out at room temperature for 23 hours. The reaction vial was taken out from the glove box. Then, ethyl vinyl ether (2.0 ml) was added to the reaction mixture. The reaction mixture was stirred for 30 minutes. A polymer solution was added to methanol (100.0 ml) to give a white polymer solid. The white solid product was collected by filtration. Then, the reprecipitation procedure in dichloromethane/methanol was repeated three times. After filtration and drying in a vacuum, the final product as a white solid in 0.34 g (68.0%) was obtained.

¹H NMR (CDCl₃) δ: 8.72 (m, br, 1H), 8.10 (m, br, 4H), 7.30 (m, br, 6H), 7.00 (m, 1H), 5.32 (m, br, 2H, 2×C═C—H), 3.85 (m, 2H, OCH₂), 3.25 to 1.00 (m, br, 7H), 1.33 (s, 9H, 3×CH₃) ppm. Anal. Calcd for C₃₄H₃₂N₄O₃: C, 74.98; H, 5.92; N, 10.29. Found: C, 74.32; H , 5.86; N, 10.16. GPC (THF): M_{w}=586000, Mₙ=73000, PDI=8.0.

### Example 17

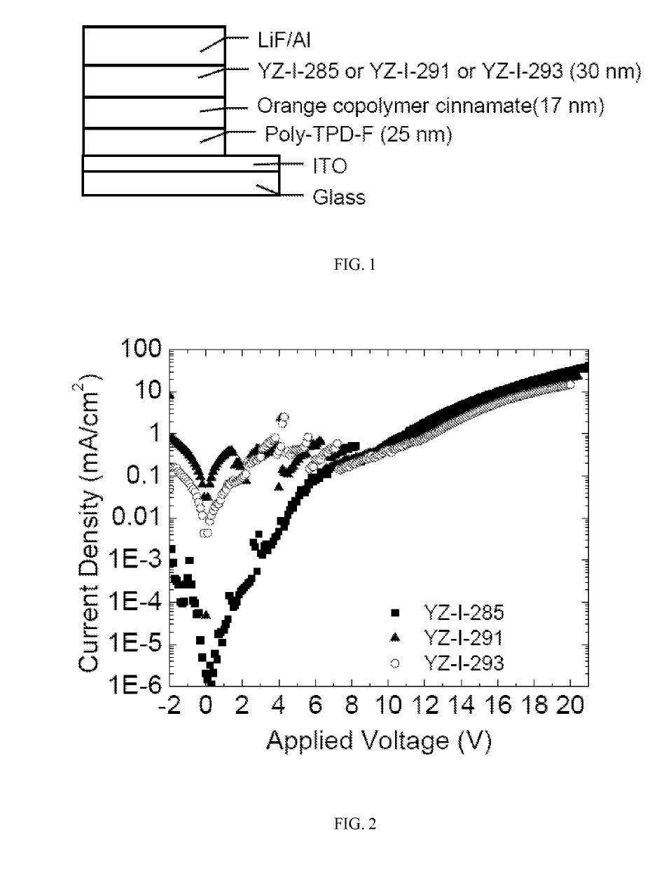

This example illustrates the formation of an OLED device using oxadiazole polymer compounds YZ-I-285 (of Example 12), YZ-I-291 (of Example 15), and YZ-I-293 (of Example 16) as a n electron transport and/or hole blocking layer. The configuration of the device is shown in FIG. 1 and is ITO/Poly-TPD-F (25 nm)/orange copolymer cinnamate (17 nm)/YZ-I-285 (of Example 12) or YZ-I-291 (of Example 15) or YZ-I-293 (of Example 16) (30 nm)/LiF/Al. Poly T-PDF and orange copolymer cinnamate are shown below:

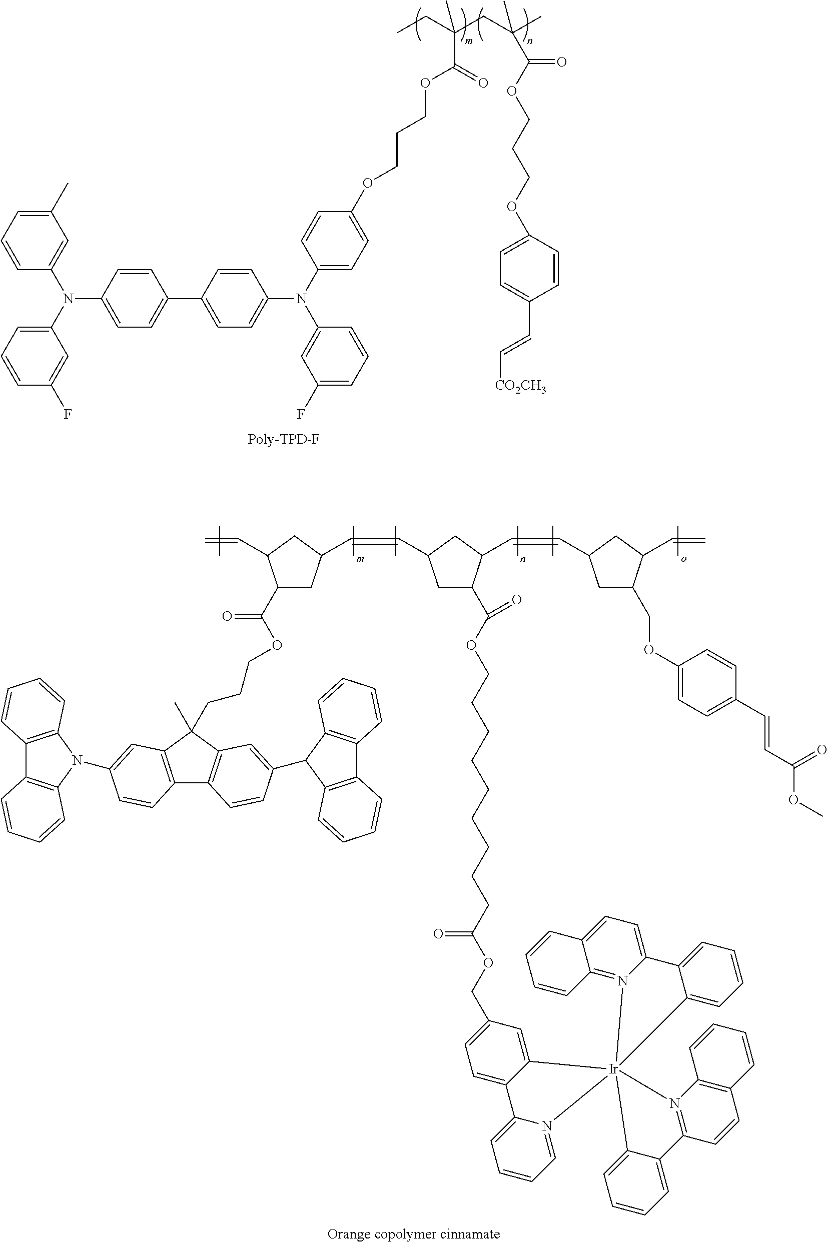

For the hole-transport layer, 10 mg of Poly-TPD-F were dissolved in 1 ml of distilled and degassed toluene. For the emissive layer, 5 mg of the cross-linkable orange copolymer with 5 mol -% Iridium content and long spacer between the Iridium complex and the polymer backbone was dissolved in 1 ml of distilled and degassed chloroform. And finally, for the electron-transport layer, 3 individual solutions of the different oxadiazole polymers were prepared by dissolving 10 mg of the oxadiazole polymers in 1 ml of distilled and degassed chlorobenzene. All solutions were stirred overnight.

25 nm thick films of the hole-transport material were spin coated (60 s@2500 rpm, acceleration 10,000) onto air plasma treated indium tin oxide (ITO) coated glass substrates with a sheet resistance of 20 ohms/sq. (Colorado Concept Coatings, L.L.C.). Films were crosslinked using a standard broad-band UV light with a 0.7 mW/cm² power density for 1 minute. Subsequently, a 17 nm thick film of the crosslinkable orange copolymer solution was spin coated on top of the crosslinked hole-transport layer (60 s@1500 rpm, acceleration 10,000). The emissive layer was crosslinked with the same UV light at 0.7 mW/cm² power density for 30 minutes. For the electron-transport layer, a 30-35 nm thick film of the oxadiazole polymer solutions was spin coated on top of the crosslinked emissive layer (60 s@1000 rpm, acceleration 10,000).

Finally, 2.5 nm of lithium fluoride (LiF) as an electron-injection layer and a 200 nm-thick aluminum cathode were vacuum deposited at a pressure below 1×10 ⁻⁶ Torr and at rates of 0.1 Å/s and 2 Å/s, respectively. A shadow mask was used for the evaporation of the metal to form five devices with an area of 0.1 cm² per substrate. At no point during fabrication, the devices were exposed to atmospheric conditions. The testing was done right after the deposition of the metal cathode in inert atmosphere without exposing the devices to air.

The performance of the above-reference compounds are shown below in Table 1.

TABLE 1
Film thickness (30 nm)
Performance of YZ-I-285, YZ-I-291 and YZ-I-293 as electron
transport and/or hole blocking layer. Averaged over four devices.

| | YZ-I-285 | YZ-I-291 | YZ-I-293 |
|---|---|---|---|
| EL efficiency (cd/A) | 4 ± 1 | 3 ± 1 | 4 ± 1 |
| External Efficiency (%) | 2.2 ± 0.1% | 1.6 ± 0.3% | 2.0 ± 0.2% |

The results are based on a luminance of 100 cd/m²

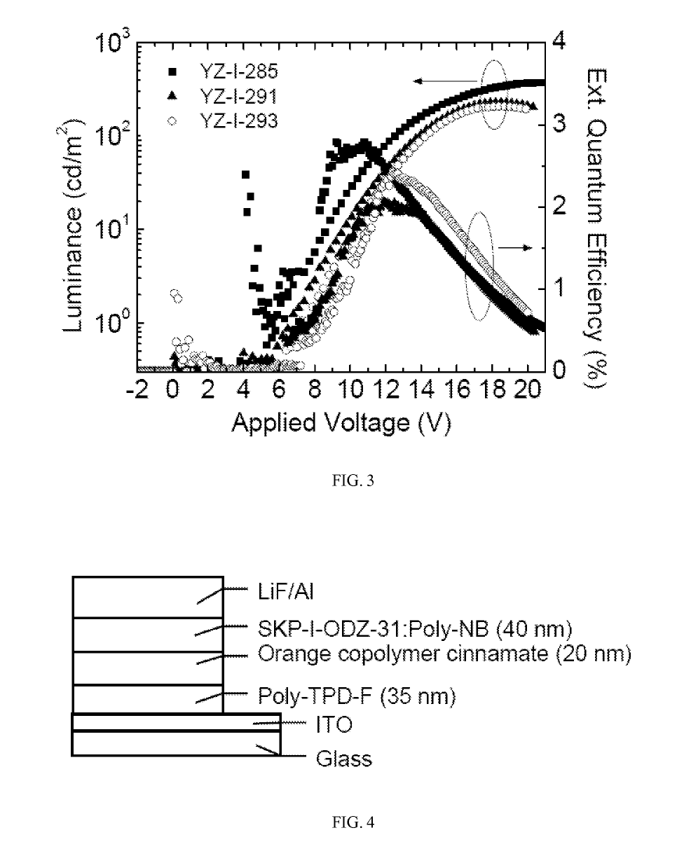

Current density-Voltage (J-V) characteristics for the above-referenced OLED devices using YZ-I-285 (of Example 12) or YZ-I-291 (of Example 15) or YZ-I-293 (of Example 16) are shown in FIG. 2 . Curves of the maximum luminance and external quantum efficiency (EQE) as a function of voltage for the above referenced OLED are shown in FIG. 3.

### Example 18

This example illustrates the formation of an OLED device using the oxadiazole compound SKP-I-ODZ-31 (example 11) mixed in the polymer Poly-NB as an electron transport and/or hole blocking layer. The configuration of the device is ITO/Poly-TPD-F (35 nm)/orange copolymer cinnamate (20 nm)/SKP-I-ODZ-3 monomer: Poly-NB (40 nm)/LiF/Al and is shown in FIG. 4. Poly-NB is shown below:

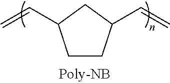

For the hole-transport layer, 10 mg of Poly-TPD-F were dissolved in 1 mL of distilled and gassed toluene. For the emissive layer, 5 mg of the crosslinkable orange copolymer with 5 mol -% Iridium content and long spacer between the Iridium complex and the polymer backbone was dissolved in 1 ml of distilled and degassed toluene. And finally, for the electron-transport layer, 9 mg of SKP-I-ODZ-31 monomer and 1 mg of Poly-NB were dissolved in 1 mL of distilled and degassed toluene. All solutions were made under inert atmosphere and were stirred overnight.

35 nm thick films of the hole transport material were spin coated (60 s@2500 rpm,acceleration 10,000) onto air plasma treated indium tin oxide (ITO) coated glass substrates with a sheet resistance of 20 ohms/sq. (Colorado Concept Coatings, L.L.C.). Films were crosslinked using a standard broad band UV light with a 0.7mW/cm² power density for 1 minute. Subsequently, a 17 nm thick film of the crosslinkable orange copolymer solution was spin coated on top of the crosslinked hole-transport layer (60 s@1500 rpm, acceleration 10,000). The emissive layer was crosslinked with the same UV light at 0.7 mW/cm² power density for 30 minutes. For the electron-transport layer, a 35 nm thick film of the oxadiazole polymer solution SKP-I-ODZ-31:Poly-NB was spin coated on top of the crosslinked emissive layer (60 s@1500 rpm, acceleration 10,000).

Finally, 2.5 nm of lithium fluoride (LiF) as an electron-injection layer and a 200 nm-thick aluminum cathode were vacuum deposited at a pressure below 1×10⁻⁶ Torr and at rates of 0.1 Å/s and 2 Å/s, respectively. A shadow mask was used for the evaporation of the metal to form five devices with an area of 0.1 cm² per substrate. At no point during fabrication, the devices were exposed to atmospheric conditions. The testing was done right after the deposition of the metal cathode in inert atmosphere without exposing the devices to air.

The performance of the above-reference compound is shown in Table 2 below.

TABLE 2
Film thickness (40 nm)
Performance of SKP-I-ODZ-31:Poly-NB as electron transport
and/or hole blocking layer. Averaged over four devices.

| | SKP-I-ODZ-31 monomer:Poly-NB |
|---|---|
| EL efficiency (cd/A) | 4 ± 1 |
| External efficiency (^{%}) | 2.7 ± 0.2% |

The results are based on a luminance of 100 cd/m²

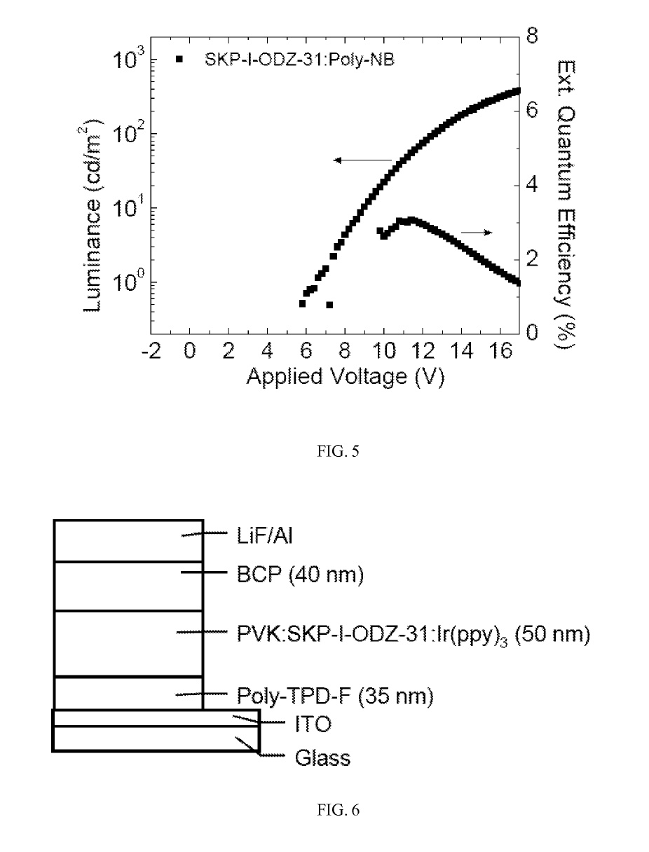

Curves of the maximum luminance and external quantum efficiency (EQE) as a function of voltage for the above referenced OLED are shown in FIG. 5.

### Example 19

This example illustrates the formation of an OLED device using the SKP-I-ODZ-31 (of Example 12) monomer compound as an electron transport material in the emissive layer. The configuration of the device is IT O/Poly-TPD-F (35 nm)/PVK: SKP-I-ODZ-31 monomer:Ir(ppy)₃ (50 nm)/BCP (40 nm)/LiF:Al and is shown in FIG. 6. PVK, Ir(ppy)₃ and BCP are shown below:

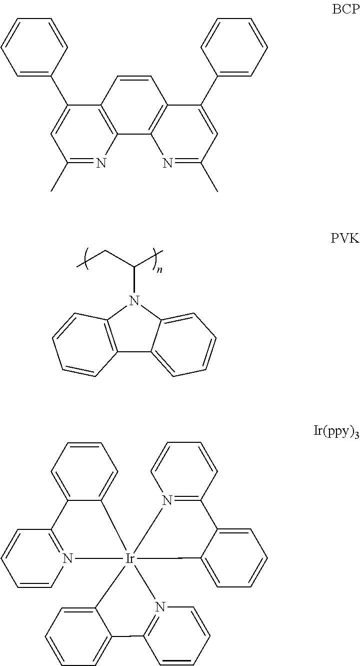

For the hole-transport layer, 10 mg of Poly-TPD-F were dissolved in 1 ml of distilled and degassed toluene. F or the emissive layer, 7 mg of the poly(N-vinyl-carbazole) (PVK), 0.6 mg of fac tris(2-phenylpyridinato-N,C²) iridium [Ir(ppy)₃] and 2.5 mg of the SKP-I-ODZ-31-monomer were dissolved in 1 mL of distilled and degassed chlorobenzene. All solutions were mad e under inert atmosphere and were stirred overnight.

35 nm thick films of the hole-transport material were spin coated (60 s@1500 rpm, acceleration 10,000) onto air plasma treated indium tin oxide (ITO) coated glass substrates with a sheet resistance 20 ohms/sq. (Colorado Concept Coatings,

L.L.C.). Films were crosslinked using a standard broad-band UV light with a 0.7 mW/cm² power density for 1 minute. Subsequently, a 50 nm thick film of the phosphorescent polymer solutions was spin coated on top of the crosslinked hole-transport layer (60 s@1000 rpm, acceleration 10,000). For the hole-blocking layer, bathocuproine (2,9-dimethyl-4,7-diphenyl-1,10-phenanthroline, BCP) was first purified using gradient zone sublimation, and a film of 40 nm was then thermally evaporated at a rate of 0.4 Å/s and at a pressure below 1×10⁻⁷ Torr on top of the emissive layer.

Finally, 2.5 nm of lithium fluoride (LiF) as an electron-injection layer and a 200 nm-thick aluminum cathode were vacuum deposited at a pressure below 1×10⁻⁶ Torr and at rates of 0.1 Å/s and 2 Å/s, respectively. A shadow mask was used for the evaporation of the metal to form five devices with an area of 0.1 cm² per substrate. At no point during fabrication, the devices were exposed to atmospheric conditions. The testing was done right after the deposition of the metal cathode in inert atmosphere without exposing the devices to air.

The performance of the above-referenced compound is shown below in Table 3.

TABLE 3
Film thickness (50 nm)
Performance of SKP-I-ODZ-31 momomer as electron transport
material in the emissive layer PVK:SKP-I-ODZ-31:Ir(ppy)₃.
Averaged over four devices.

| | PVK:SKP-I-ODZ-31:Ir(ppy)₃ |
|---|---|
| EL efficiency (cd/A) | 32 ± 1 |
| External efficiency (%) | 9.5 ± 0.3% |

The results are based on a luminance of 1,000 cd/m²

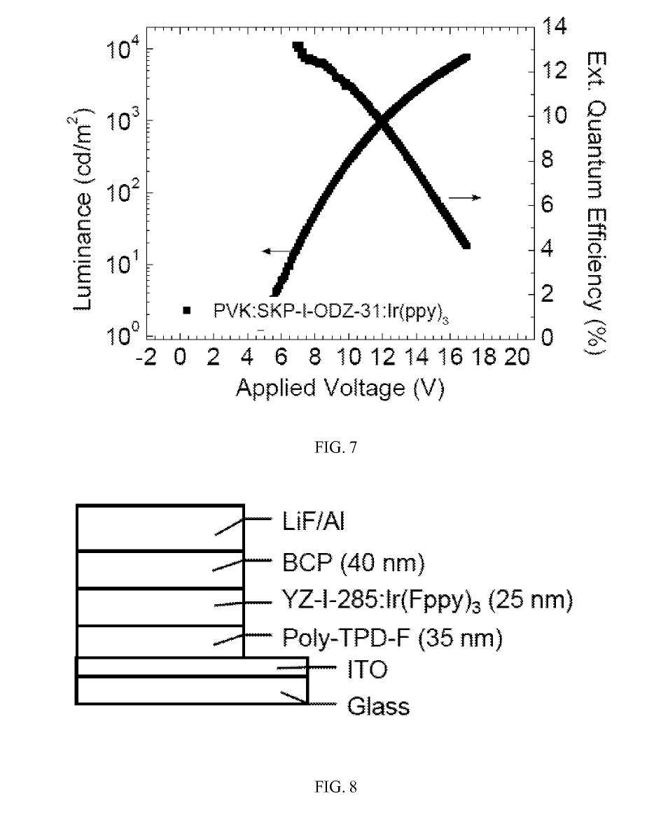

Curves of the maximum luminance and external quantum efficiency (EQE) as a function of voltage for the above referenced OLED are shown in FIG. 7.

### Example 20

This example illustrates the formation of an OLED device using an oxadiazole polymer compound as a host in the emissive layer. The configuration of the device is ITO/Poly-TPD-F (35 nm)/YZ-I-285:Ir(Fppy)₃ (25 nm)/BCP (40 nm)/LiF:Al and is shown in FIG. 8. Ir(Fppy)₃ is shown below:

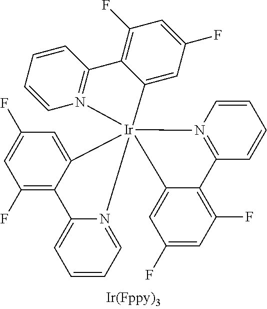

For the hole-transport layer, 10 mg of Poly-TPD-F were dissolved in 1 mL of distilled and degassed toluene. For the emissive layer, 9 mg of YZ-I-285 and 1 mg offac-tris-4,6 difluorophenylpyridine Iridium(III) [Ir(Fppy)₃] were dissolved in 1 mL of distilled and degassed chlorobenzene. All solutions were made under inert atmosphere and were stirred overnight.

35 nm thick films of the hole-transport material were spin coated (60 s@1500 rpm, acceleration 10,000) onto air plasma treated indium tin oxide (ITO) coated glass substrates with a sheet resistance of 20 ohms/sq. (Colorado Concept Coatings, L.L.C.). Films were crosslinked using a standard broad-band UV light with a 0.7 mW/cm² power density for 1 minute. Subsequently, a 25 nm thick film of the phosphorescent polymer solutions was spin coated on top of the crosslinked hole-transport layer (60 s@1500 rpm, acceleration 10,000). For the hole-blocking layer, bathocuproine (2,9-dimethyl-4,7-diphenyl-1,10-phenanthroline, BCP) was first purified using gradient zone sublimation, and a film of 40 nm was then thermally evaporated at a rate of 0.4 Å/s and at a pressure below 1×10⁻⁷ Torr on top of the emissive layer.

Finally, 2.5 nm of lithium fluoride (LiF) as an electron-injection layer and a 200 nm-thick aluminum cathode were vacuum deposited at a pressure below 1×10 ⁻⁶ Torr and at rates of 0.1 Å/s and 2 Å/s, respectively. A shadow mask was used for the evaporation of the metal to form five devices with an area of 0.1 cm² per substrate. At no point during fabrication were the devices exposed to atmospheric conditions. The testing was done right after the deposition of the metal cathode in inert atmosphere without exposing the devices to air.

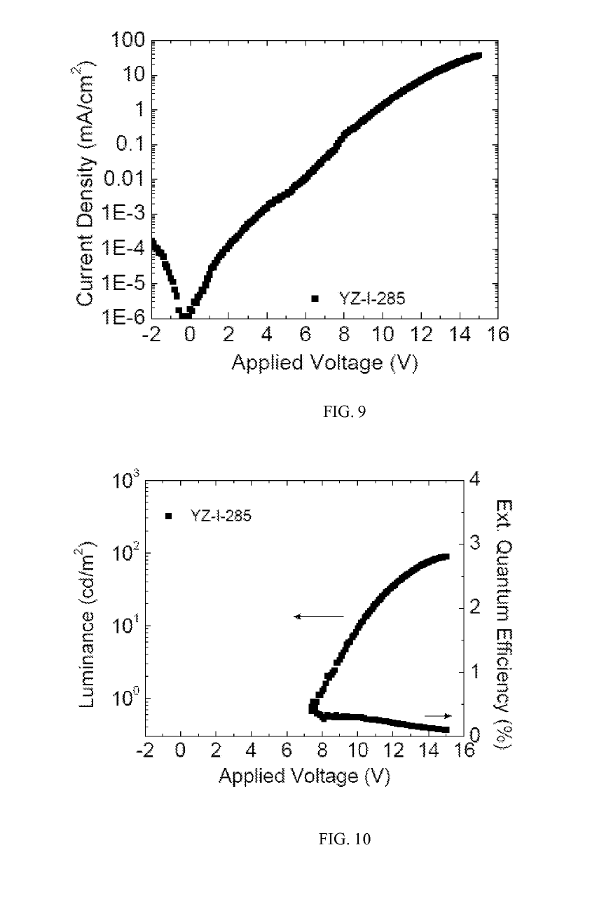

Current density-Voltage (J-V) characteristics for the above-referenced OLED devices using YZ-I-285:Ir(Fppy)₃ as an emissive layer is shown in FIG. 9. Curves of the maximum luminance and external quantum efficiency (EQE) as a function of voltage for the above referenced OLED are shown in FIG. 10.

### Example 21

This example illustrates the formation of an OLED device using the polymer YZ-I-293 (of Example 16) as an electron transport material in the emissive layer with the polymer PVK as a hole transport material and compound Ir(ppy)₃ as an emitter. The configuration of the device is ITO/Poly-TPD-F (35 nm)/PVK: YZ-I-293: Ir(ppy)₃ (40 nm)/BCP (40 nm)/LiF:Al and is shown in FIG. 11.

For the hole-transport layer, 10 mg of Poly-TPD-F were dissolved in 1 ml of distilled and degassed toluene. For the emissive layer, 4.4 mg of the poly(N-vinyl-carbazole) (PVK), 0.6 mg of fac tris(2-phenylpyridinato-N,C²) iridium [Ir(ppy)₃] and 5.0 mg of YZ-I-293 were dissolved in 1 ml of distilled and degassed chlorobenzene. All solutions were made under inert atmosphere and were stirred overnight.

35 nm thick films of the hole-transport material were spin coated (60 s@1500 rpm, acceleration 10,000) onto air plasma treated indium tin oxide (ITO) coated glass substrates with a sheet resistance of 20 ohms/sq. (Colorado Concept Coatings, L.L.C.). Films were crosslinked using a standard broad-band UV light with a 0.7 mW/cm² power density for 1 minute. Subsequently, a 40 nm thick film of the phosphorescent polymer solutions was spin coated on top of the cross linked hole-transport layer (60 s@1000 rpm, acceleration 10,000). For the hole-blocking layer, bathocuproine (2,9-dimethyl-4,7-diphenyl-1,10-phenanthroline, BCP) was first purified using gradient zone sublimation, and a film of 40 nm was then thermally evaporated at a rate of 0.4 Å/s and at a pressure below 1×10⁻⁷ Torr on top of the emissive layer.

Finally, 2.5 nm of lithium fluoride (LiF) as an electron-injection layer and a 200 nm-thick aluminum cathode were vacuum deposited at a pressure below 1×10⁻⁶ Torr and at rates of 0.1 Å/s and 2 Å/s, respectively. A shadow mask was used for the evaporation of the metal to form five devices with an area of 0.1 cm² per substrate. At no point during fabrication, the devices were exposed to atmospheric conditions. The testing was done right after the deposition of the metal cathode in inert atmosphere without exposing the devices to air.

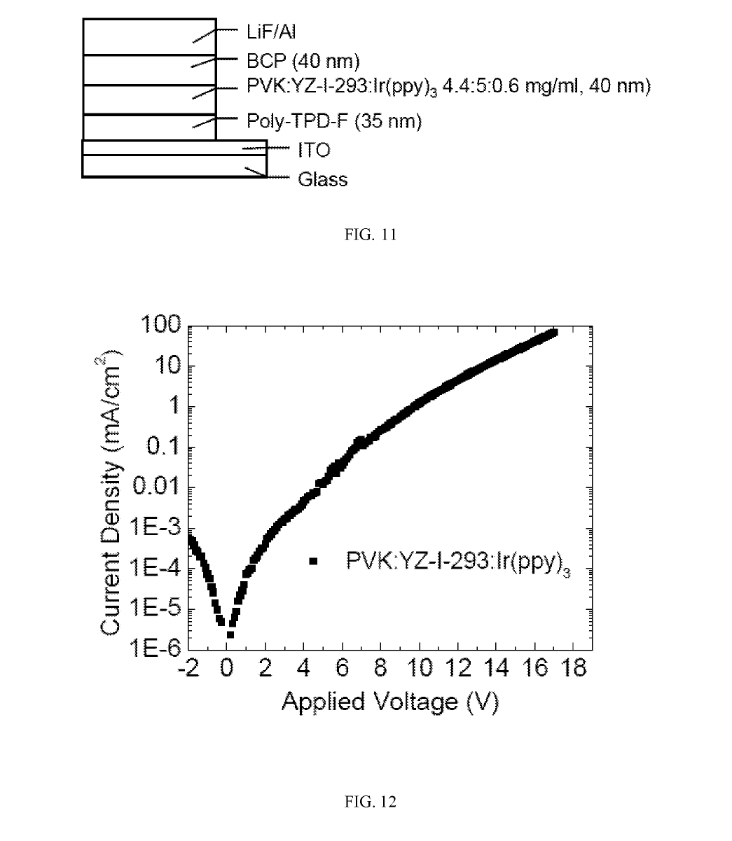

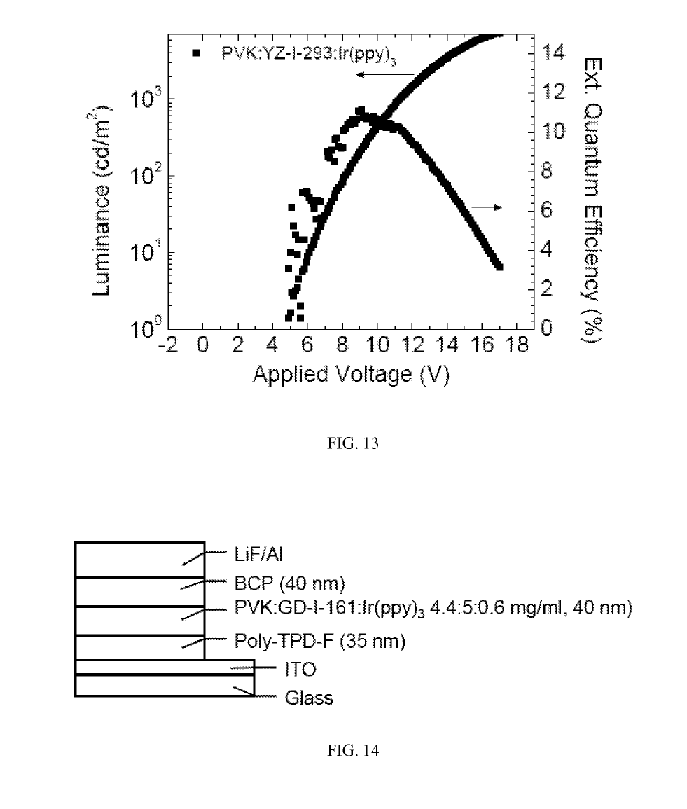

Current density-Voltage (J-V) characteristics for the above-referenced OLED devices using PVK:YZ-I-293:Ir(ppy)₃ as an emissive layer is shown in FIG. 12. Curves of the maximum luminance and external quantum efficiency (EQE) as a function of voltage for the above referenced OLED are shown in FIG. 13.

### Example 22

This example illustrates the formation of an OLED device using the polymer GD-I-161 (of Example 23) as an electron transport material in the emissive layer with polymer PVK as a hole transport material and compound Ir(ppy)₃ as an emitter. The configuration of the device is ITO/Poly-TPD-F (35 nm)/PVK:GD-I-161:Ir(ppy)₃ (40 nm)/BCP (40nm)/LiF:Al and is shown in FIG. 14. The structure of GD-I-161 is shown below:

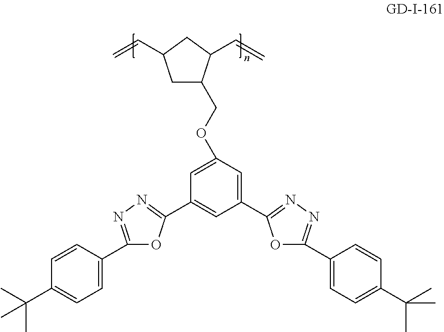

For the hole-transport layer, 10 mg of Poly-TPD-F were dissolved in 1 ml of distilled and degassed toluene. For the emissive layer, 4.4 mg of the poly(N-vinyl-carbazole) (PVK), 0.6 mg of fac tris(2-phenylpyridinato-N,C²′) iridium [Ir(ppy)₃] and 5.0 mg of GD-I-161 (see Example 23) were dissolved in 1 ml of distilled and degassed chlorobenzene. All solutions were made under inert atmosphere and were stirred overnight.

35 nm thick films of the hole-transport material were spin coated (60 s@1500 rpm, acceleration 10,000) onto air plasma treated indium tin oxide (ITO) coated glass substrates with a sheet resistance of 20 ohms/sq. (Colorado Concept Coatings, L.L.C.). Films were crosslinked using a standard broad-band UV light with a 0.7 mW/cm² power density for 1 minute. Subsequently, a 40 nm thick film of the emissive phosphorescent polymer solutions was spin coated on top of the crosslinked hole-transport layer (60 s@1000 rpm, accelerati on 10,000). For the hole-blocking layer, bathocuproine (2,9-dimethyl-4,7-diphenyl-1,10-phenanthroline, BCP) was first purified using gradient zone sublimation, and a film of 40 nm was then thermally evaporated at a rate of 0.4 Å/s and at a pressure below 1×10⁻⁷ Torr on top of the emissive layer.

Finally, 2.5 nm of lithium fluoride (LiF) as an electron-injection layer and a 200 nm-thick aluminum cathode were vacuum deposited at a pressure below 1×10⁻⁶

Torr and at rates of 0.1 Å/s and 2 Å/s, respectively. A shadow mask was used for the evaporation of the metal to form five devices with an area of 0.1 cm² per substrate. At no point during fabrication, the devices were exposed to atmospheric conditions. The testing was done right after the deposition of the met al cathode in inert atmosphere without exposing the devices to air.

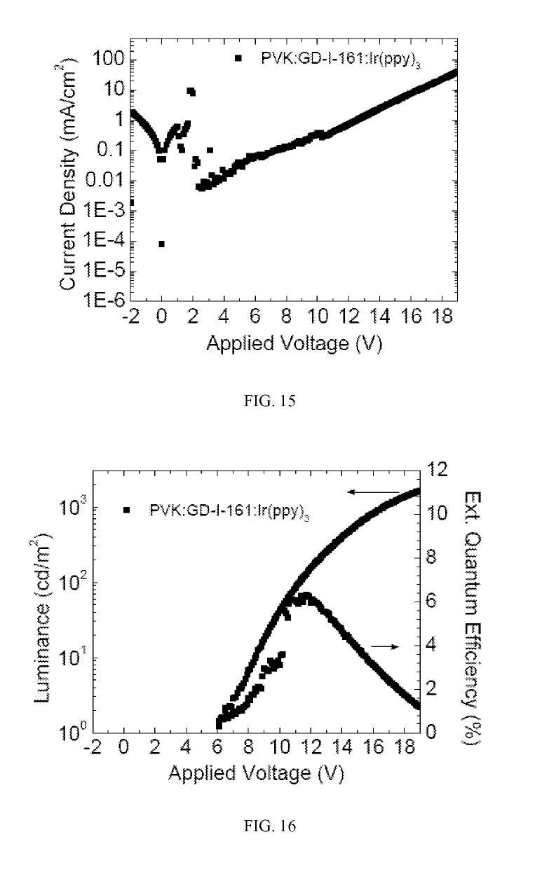

Current density-Voltage (J-V) characteristics for the above-referenced OLED devices using PVK: GD-I-161:Ir(ppy)₃ as an emissive layer is shown in FIG. 15. Curves of the maximum luminance an d external quantum efficiency (EQE) as a function of voltage for the above referenced OLED are shown in FIG. 16.

### Example 23

Poly(5-(Bicyclo[2,21]hept-5-en-2-ylmethoxy)-1,3-phenylene)bis(2-(4-tert-butylphenyl)-1,3,4-oxadiazole (GD-I-161)

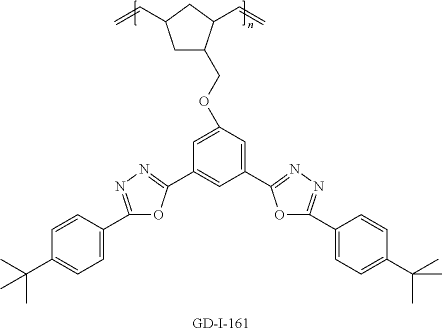

Polymer GD-I-161 was prepared from monomer YZ-I-259 (see Example 2) by the following procedure.

5,5′-(5-(Bicyclo [2,21]hept-5-en-2-ylmethoxy)-1,3-phenylene)bis(2-(4-tert-butylphenyl)-1,3,4-oxadiazole (0.35 g, 0.583 mmol) (YZ-I-259, see Example 2), and a 1^{st} generation Grubb s catalyst (4.8 mg, 0.0058 mmol) were mixed well in CH₂Cl₂ (12.0 ml) at room temperature, under stirring in a glove box. The reaction was carried out at room temperature for 23 hours. The reaction vial was taken out from the glove box. Then, ethyl vinyl ether (2.0 ml) was added to the reaction mixture. The reaction mixture was stirred for 1 hour. A polymer solution was dropped into methanol (75.0 ml) to give a white polymer solid. The white solid product was collected by filtration. The reprecipitation procedure in dichloromethane/methanol was then repeated five times. After filtration and drying in a vacuum, the final product as a white solid was obtained in 0.20 g (60.0%) yield.

¹H NMR (CDCl₃) δ: 8.10 (m, br, 6H), 7.60-6.80 (m, br, 6H), 5.40 (m, br, 2H, 2×C═C—H), 4.13 (m, br, 2H, OCH₂), 3.25-1.00 (m, br, 7H), 1.34 (s, br, 18H, 6×CH₃) ppm. GPC (CHCl₃): M_{w}=125000 Mₙ=67000, PDI=1.5.

While this invention has been described in terms o f what are presently considered to be the most practical and preferred embodiments, it is to be understood that the invention need not be limited to the enclosed embodiment. To the contrary, it is intended to cover various modifications and similar arrangements included within the spirit and scope of the appended claims which are to be accorded with the broadest interpretation so as to encompass all such modifications and similar structures. Therefore, the above description and illustration should not be taken as limiting the scope of the present invention which is defined by the appended claims.

1. A compound represented by the formula: 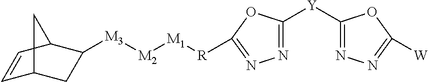
wherein:
R and W are independently selected arenes comprising six to twenty carbon atoms optionally substituted with 1, 2, or 3 independently selected alkyl or alkoxy groups,
Y is absent or is C₆-C₂₀ arene,
wherein;
M₁ and M₃ are optional or independently selected from 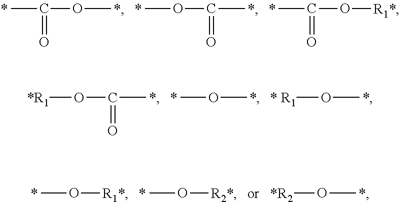
and M₁ and M₃ are bound to the norbornene or R at the positions indicated by *;
R₁ and R₂ are optional independently selected C₁₋₂₀ alkane diyl, alkene diyl, alkyne diyl, or arene diyl groups;
and optional M₂ is a C₁₋₂₀ alkane diyl, alkene diyl, alkyne diyl, or arene diyl group.

2. The compound of claim 1, wherein Y is a phenyl, naphthyl, anthracenyl, fluorenyl, phenanthrenyl, pyridyl, or biphenyl which is optionally substituted with C₁₋₁₂ alkyl or alkoxy groups.

3. The compound of claim 1, having the structure:

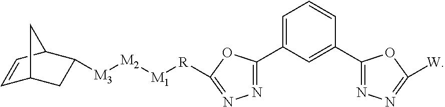

4. (canceled)

5. The compound of claim 1 having the structure: 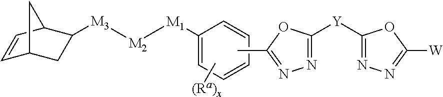
wherein:
each optional R^{a} group is independently selected from one or more C₁₋₂₀ alkyl, or alkoxy groups, and x is an integer 1, 2, or 3.

6. (canceled)

7. The compound of claim 1 having the structure: 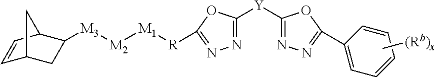
wherein:
each optional R^{b} group is independently selected from one or more C₁₋₂₀ alkyl or alkoxy groups,
and x is an integer 1, 2, or 3.

8. (canceled)

9. (canceled)

10. (canceled)

11. (canceled)

12. (canceled)

13. (canceled)

14. The compound of claim 1, wherein M₃-M₂-M₁ is 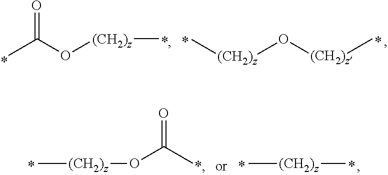
where z and z′ are independently selected integers 0, 1, 2, 3, 4, 5, 6, 7, 8, 9 or 10.

15. (canceled)

16. (canceled)

17. (canceled)

18. (canceled)

19. (canceled)

20. A process for preparing a polymer or copolymer comprising a) mixing at least one monomeric compound of claim 1 with a ring opening metathesis catalyst, and b) polymerizing the mixture to form a polymer comprising at least some polynorbornenyl repeat units having the structure:

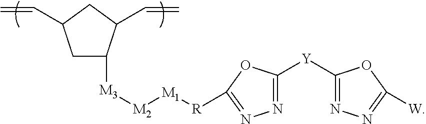

21. (canceled)

22. The polymer or copolymer product produced by the process of claim 20.

23. (canceled)

24. A polymer represented by the formula: 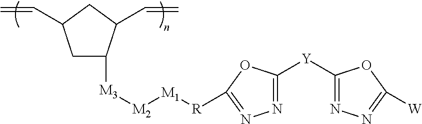
wherein:
R and W are independently selected arenes comprising six to twenty carbon atoms and optionally substituted with 1, 2, or 3 independently selected alkyl or alkoxy groups,
Y is absent or is C₆-C₂₀arene,
n is an integer from 5 to 2000,
wherein;
M₁ and M₃ are optional or independently selected from 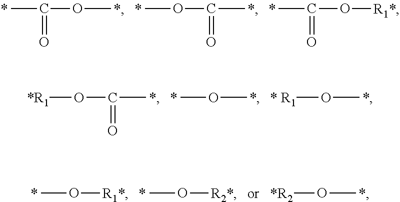
and M₁ and M₃ are bound to the norbornene or R at the positions indicated by *;
R₁ and R₂ are optional independently selected C₁-₂₀ alkane diyl, alkene diyl, alkyne diyl, or arene diyl groups;
and optional M₂ is a C₁₋₂₀ alkane diyl, alkene diyl, alkyne diyl, or arene diyl group.

25. The polymer of claim 24, wherein Y is a phenyl, naphthyl, anthracenyl, fluorenyl, phenanthrenyl, pyridyl or biphenyl which is optionally substituted with 1, 2, or 3 alkyl or alkoxy groups.

26. The polymer of claim 24 having the structure:

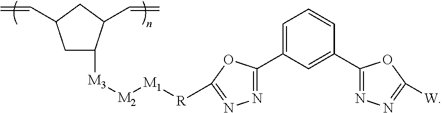

27. (canceled)

28. The polymer of claim 24 having the structure: 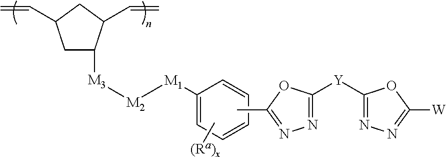
wherein:
each optional R^{a} group is independently selected from one or more C₁₋₂₀ alkyl, or alkoxy groups, and
x is an integer 1, 2, or 3.

29. (canceled)

30. The polymer of claim 24 having the structure: 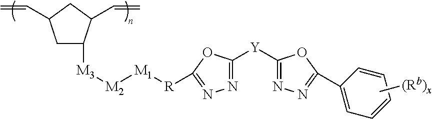
wherein:
each optional R^{b} group is independently selected from one or more C₁₋₂₀ alkyl or alkoxy groups, and x is an integer 1, 2, or 3.

31. (canceled)

32. (canceled)

33. (canceled)

34. (canceled)

35. The polymer of claim 24, wherein M₂ is absent or is —(CH₂)_{z}—, where z is an integer 1, 2, 3, 4, 5, 6, 7, 8, 9, 10, or 11.

36. (canceled)

37. The polymer of claim 24, wherein M₃-M₂-M₁ is 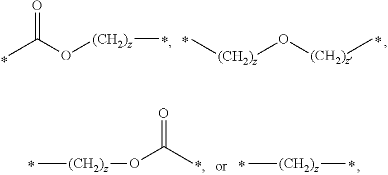
where z and z′ are independently selected integers 0, 1, 2, 3, 4, 5, 6, 7, 8, 9 or 10.

38. (canceled)

39. (canceled)

40. The polymer of claim 24, having the structure: 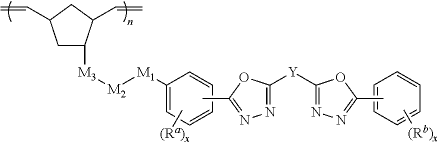
wherein:
each optional R^{a} or R^{b} group is independently selected from one or more C₁₋₂₀ alkyl or alkoxy groups, and
each x is an independently selected integer 0, 1, 2, 3 or 4.

41. (canceled)

42. (canceled)

43. (canceled)

44. A device comprising at least one polymer of claim 24.

45. (canceled)

46. (canceled)

47. (canceled)

48. An organic electroluminescence device an electron transport layer or a light-emitting layer is comprising a poly(norbornene) homopolymer, or a poly(norbornene) copolymer compound of the polymer of claim 24.

49. The compound of claim 1 being selected from the group consisting of:

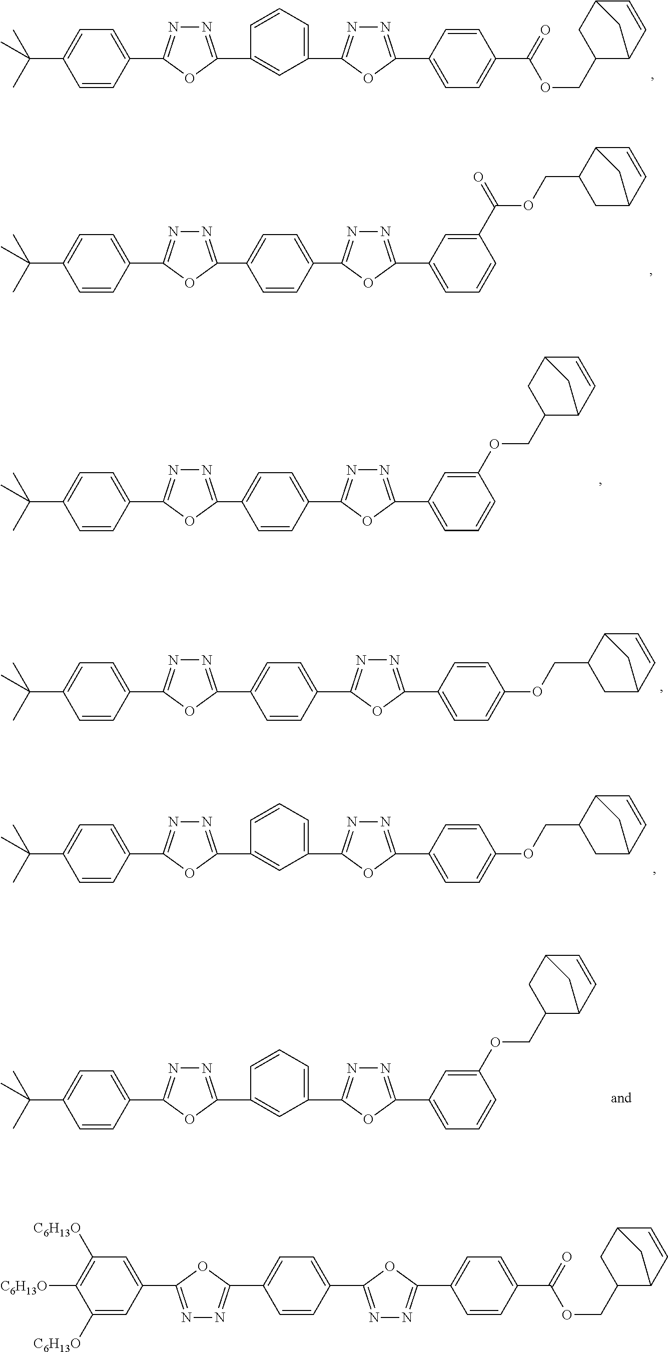

50. The polymer of claim 24 being selected from the group consisting of: 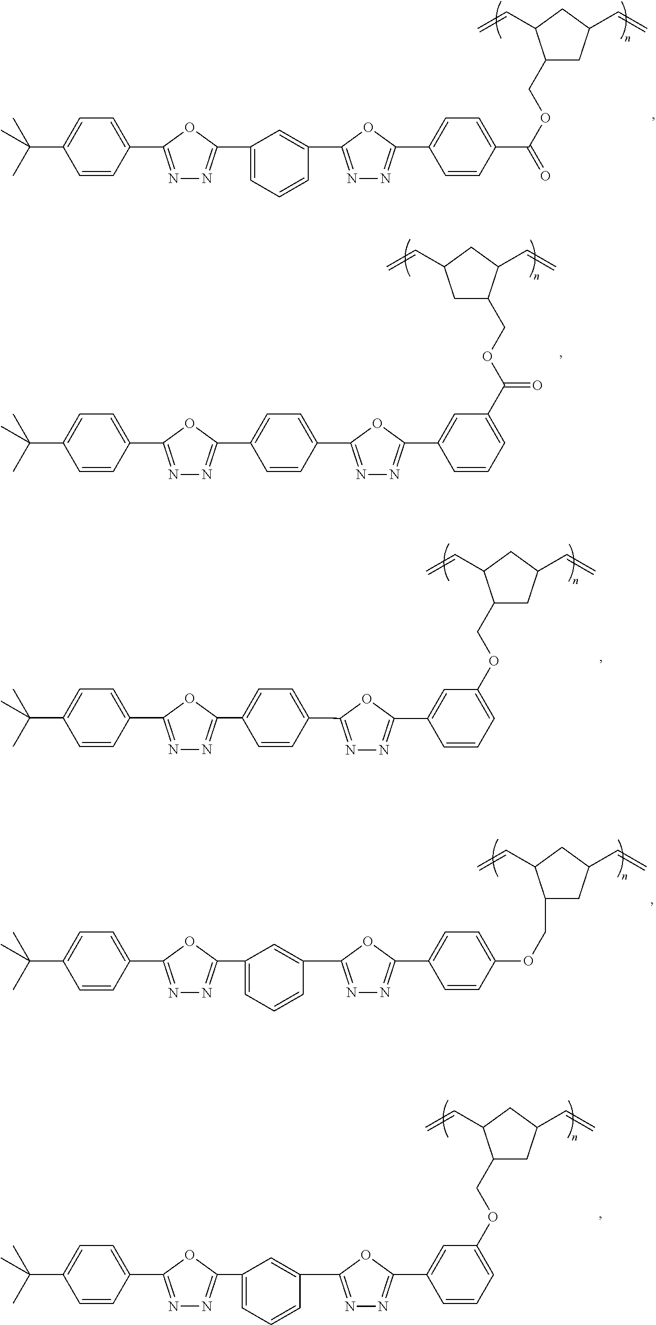 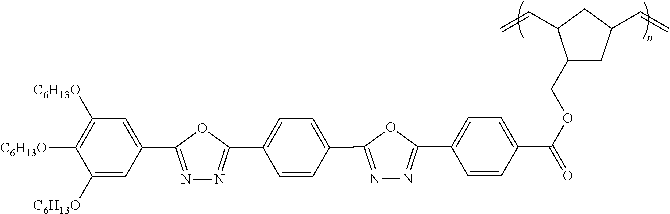
and mixtures thereof.

51-59. (canceled)
